(19) United States
(12) Patent Application Publication
(10) Pub. No.: US 2024/0382174 A1
(43) Pub. Date: Nov. 21, 2024

(54) SURGICAL VISUALIZATION SYSTEMS

(71) Applicant: CAMPLEX, INC., GERMANTOWN, TN (US)

(72) Inventor: John Tesar, Tucson, AZ (US)

(21) Appl. No.: 18/518,045

(22) Filed: Nov. 22, 2023

Related U.S. Application Data
(63) Continuation of application No. 17/808,056, filed on Jun. 21, 2022, now abandoned.
Application No. 17/808,056 is a continuation of application No. 14/581,779, filed on Dec. 23, 2014, now abandoned.
(60) Provisional application No. 61/920,451, filed on Dec. 23, 2013.
(60) Provisional application No. 61/921,051, filed on Dec. 26, 2013.
(60) Provisional application No. 61/921,389, filed on Dec. 27, 2013.
(60) Provisional application No. 61/922,068, filed on Dec. 30, 2013.
(60) Provisional application No. 61/923,188, filed on Jan. 2, 2014.
(60) Provisional application No. 62/088,470, filed on Dec. 5, 2014.

(51) Int. Cl. A61B 8/13; A61B 1/05; A61B 5/055; A61B 6/03; A61B 17/00; A61B 17/02; A61B 17/16; A61B 34/20; A61B 90/00; A61B 90/20; A61B 90/30; G02B 21/00; G02B 21/16; G02B 21/22; G06T 7/30

(52) U.S. Cl. CPC A61B 8/13; A61B 1/05; A61B 5/055; A61B 6/032; A61B 17/0206; A61B 17/1611; A61B 90/20; A61B 90/361; A61B 90/37; G02B 21/0012; G02B 21/16; G02B 21/22; G06T 7/30; A61B 2017/00535; A61B 2034/2048; A61B 2034/2051; A61B 2034/2055; A61B 2090/306; A61B 2090/364; A61B 2090/371; G06T 2207/10064; G06T 2207/10068; G06T 2207/10081; G06T 2207/10132; G06T 2207/20221

(57) ABSTRACT

A medical apparatus is described for providing visualization of a surgical site. The medical apparatus includes an electronic display disposed within a display housing, the electronic display configured to produce a two-dimensional image. The medical apparatus includes a display optical system disposed within the display housing, the display optical system comprising a plurality of lens elements disposed along an optical path. The display optical system is configured to receive the two-dimensional image from the electronic display, produce a beam with a cross-section that remains substantially constant along the optical path, and produce a collimated beam exiting the opening in the display housing. The medical apparatus can also include an auxiliary video camera configured to provide an oblique view of a patient on the electronic display without requiring a surgeon to adjust their viewing angle through oculars viewing the electronic display.

### CROSS-REFERENCE TO RELATED APPLICATIONS

This application is a continuation of U.S. patent application Ser. No. 17/808,056, entitled “SURGICAL VISUALIZATION SYSTEMS”, filed Jun. 21, 2022, which is a continuation of U.S. patent application Ser. No. 14/581,779, entitled “SURGICAL VISUALIZATION SYSTEMS”, filed Dec. 23, 2014, which claims the benefit of priority to U.S. Prov. App. No. 61/920,451, entitled “SURGICAL VISUALIZATION SYSTEMS”, filed Dec. 23, 2013; to U.S. Prov. App. No. 61/921,051, entitled “SURGICAL VISUALIZATION SYSTEMS”, filed Dec. 26, 2013; to U.S. Prov. App. No. 61/921,389, entitled “SURGICAL VISUALIZATION SYSTEMS”, filed Dec. 27, 2013; to U.S. Prov. App. No. 61/922,068, entitled “SURGICAL VISUALIZATION SYSTEMS”, filed Dec. 30, 2013; to U.S. Prov. App. No. 61/923,188, entitled “SURGICAL VISUALIZATION SYSTEMS”, filed Jan. 2, 2014; and to U.S. Prov. App. No. 62/088,470, entitled “SURGICAL VISUALIZATION SYSTEMS AND DISPLAYS”, filed Dec. 5, 2014. Each of the applications cited in this paragraph is incorporated by reference herein in its entirety.

### BACKGROUND

### Field

Embodiments of the present disclosure relate to visualization systems and displays for use during surgery.

### Description of Related Art

Some surgical operations involve the use of large incisions. These open surgical procedures provide ready access for surgical instruments and the hand or hands of the surgeon, allowing the user to visually observe and work in the surgical site, either directly or through an operating microscope or with the aide of loupes. Open surgery is associated with significant drawbacks, however, as the relatively large incisions result in pain, scarring, and the risk of infection as well as extended recovery time. To reduce these deleterious effects, techniques have been developed to provide for minimally invasive surgery. Minimally invasive surgical techniques, such as endoscopy, laparoscopy, arthroscopy, pharyngo-laryngoscopy, as well as small incision procedures utilizing an operating microscope for visualization, utilize a significantly smaller incision than typical open surgical procedures. Specialized tools may then be used to access the surgical site through the small incision. However, because of the small access opening, the surgeon's view and workspace of the surgical site is limited. In some cases, visualization devices such as endoscopes, laparoscopes, and the like can be inserted percutaneously through the incision to allow the user to view the surgical site.

The visual information available to a user without the aid of visualization systems and/or through current laparoscopic or endoscopic systems contain trade-offs in approach. Accordingly, there is a need for improved visualization systems, for use in open and/or minimally invasive surgery.

### SUMMARY

The systems, methods and devices of the disclosure each have innovative aspects, no single one of which is solely responsible for the desirable attributes disclosed herein.

In a first aspect, a medical apparatus is provided that includes a display housing and an opening in the display housing. The medical apparatus also includes an electronic display disposed within the display housing, the electronic display comprising a plurality of pixels configured to produce a two-dimensional image. The medical apparatus also includes a display optical system disposed within the display housing, the display optical system comprising a plurality of lens elements disposed along an optical path. The display optical system is configured to receive the two-dimensional image from the electronic display, produce a beam with a cross-section that remains substantially constant along the optical path, and produce a collimated beam exiting the opening in the display housing.

In some embodiments of the first aspect, the display optical system further comprises an optical redirection element configured to fold the optical path. In a further embodiment of the first aspect the optical redirection element comprises a mirror or a prism. In another embodiment of the first aspect, the display optical system is configured to direct light received from the electronic display to the opening in the display housing while reducing stray light.

In some embodiments of the first aspect, the display optical system further comprises a baffle configured to reduce stray light. In a further embodiment, the display optical system comprises less than or equal to four baffles. In a further embodiment, the display optical system comprises less than or equal to four mirrors. In a further embodiment, a first baffle is positioned between the electronic display and a first baffle along the optical path, the first mirror positioned prior to the plurality of lens elements along the optical path from the display to the opening. In another further embodiment, at least three baffles are positioned prior to the plurality of lens elements along the optical path from the display to the opening. In another further embodiment, at least two mirrors are positioned prior to the plurality of lens elements along the optical path from the display to the opening.

In some embodiments of the first aspect, the display optical system has an exit pupil and the electronic display is not parallel to the exit pupil. In some embodiments of the first aspect, the opening in the display housing comprises a mounting interface configured to mate with a binocular assembly for a surgical microscope. In a further embodiment, an exit pupil of the display optical system is of a same size or smaller than an entrance pupil of oculars in the binocular assembly.

In some embodiments of the first aspect, the medical apparatus further comprises a second electronic display and a second display optical system configured to provide a stereo view. In some embodiments of the first aspect, the medical apparatus further comprises processing electronics configured to communicate with the electronic display to provide images for the electronic display. In a further embodiment, the processing electronics are configured to receive images from one or more cameras on a surgical device. In a further embodiment, the processing electronics are configured to receive images from one or more cameras that provide a surgical microscope view.

In some embodiments of the first aspect, the optical path is less than or equal to 16.2 inches and a light-emitting portion of the electronic display has a diagonal measurement that is greater than or equal to 5 inches. In some embodiments of the first aspect, the optical path is less than or equal to 18.7 inches and a light-emitting portion of the electronic display has a diagonal measurement that is greater than or equal to 8 inches. In some embodiments of the first aspect, the display optical system further comprises a converging mirror. In some embodiments of the first aspect, the medical apparatus further comprises a viewing assembly comprising an objective lens, beam positioning optics, and an ocular, the viewing assembly configured to receive the collimated beam exiting the opening in the display housing. In some embodiments of the first aspect, the electronic display has a diagonal light-emitting portion between 4 inches and 9 inches. In some embodiments of the first aspect, an optical path length from the electronic display to a last element of the display optical system is at least 9 inches. In a further embodiment, the optical path length from the electronic display to the last element of the display optical system is less than 20 inches.

In a second aspect, a medical apparatus is provided that includes a viewing assembly comprising a housing and an ocular, the ocular configured to provide a view an electronic display disposed in the housing. The medical assembly includes an optical assembly disposed on the viewing assembly, the optical assembly configured to provide a surgical microscope view of a surgical site. The optical assembly includes an auxiliary video camera and a gimbal configured to couple the auxiliary video camera to the viewing assembly and configured to change an orientation of the auxiliary video camera relative to the viewing assembly. The medical apparatus includes an image processing system in communication with the optical assembly and the electronic display, the image processing system comprising processing electronics. The image processing system is configured to receive video images acquired by the auxiliary video camera, provide output video images based on the received video images, and present the output video images on the electronic display so that the output video images are viewable through the ocular. The gimbal is configured to adjust a pitch of the auxiliary video camera between a first position and a second position, wherein the auxiliary video camera has a first viewing angle perpendicular to a floor in the first position and a second viewing angle that is within about 10 degrees of parallel to the floor in the second position.

In some embodiments of the second aspect, the gimbal comprises two pivots. In a further embodiment, a first pivot is configured to adjust a pitch of the auxiliary video camera and a second pivot is configured to rotate the auxiliary video camera around an axis perpendicular to the floor.

In some embodiments of the second aspect, the gimbal is configured to adjust a pitch of the auxiliary video camera between the first position and a third position, wherein the auxiliary video camera has a third viewing angle in the third position that is less than or equal to 180 degrees from the first viewing angle. In some embodiments of the second aspect, the gimbal is electronically controlled. In some embodiments of the second aspect, the optical assembly is configured to provide an oblique view of a portion of a patient. In a further embodiment, an orientation of the ocular of the viewing assembly is configured to remain stationary when an orientation of the auxiliary video camera changes to provide the oblique view of the portion of the patient.

In some embodiments of the second aspect, the gimbal is configured to smoothly adjust the viewing angle of the auxiliary video camera between the first position and the second position. In some embodiments of the second aspect, the auxiliary video camera comprises a stereo video camera and the ocular comprises a pair of oculars. In some embodiments of the second aspect, the medical apparatus further comprises a camera arm attached to the viewing assembly.

In a third aspect, a medical apparatus is provided that includes a display housing. The medical apparatus includes a plurality of electronic displays disposed within the display housing, each of the plurality of electronic displays comprising a plurality of pixels configured to produce a two-dimensional image. The plurality of electronic displays is configured to present superimposed images in a field of view of a person's eye.

In some embodiments of the third aspect, the medical apparatus further comprises a binocular viewing assembly coupled to the display housing. In some embodiments of the third aspect, at least one of the plurality of electronic displays comprises a transmissive display panel. In some embodiments of the third aspect, the superimposed images comprise a video of a first portion of a surgery site that is superimposed on a video of a second portion of the surgery site, the first portion contained within the second portion. In a further embodiment, the video of the first portion is magnified relative to the video of the second portion.

In some embodiments, a medical apparatus can include a camera having a field of view that can be designed to include a surgical site, wherein the camera is designed to provide a surgical microscope view of the surgical site. In some embodiments, the medical apparatus can include a binocular viewing assembly having a housing and a plurality of oculars, the plurality of oculars designed to provide views of at least one display disposed in the housing. In some embodiments, the medical apparatus can include an image processing system designed to receive images acquired by the camera and present the output video images on the at least one display. In some embodiments, the medical apparatus can include a movement control system designed to move the camera relative to the binocular viewing assembly, the movement control system having a control member operatively coupled to the movement control system to translate the camera relative to the binocular viewing assembly along at least a first axis and a second axis and to rotate the camera relative to the binocular viewing assembly.

In a fourth aspect a medical apparatus is provided wherein a movement control system can include a translation system having a moveable platform to which the camera is attached, the moveable platform being positioned between the binocular viewing assembly and the camera and being moveable relative to the binocular viewing assembly along at least a first axis and a second axis. In some embodiments, the translation system can include an electromechanical device operatively coupled to the moveable platform.

In some embodiments of the fourth aspect, the movement control system can include a pitch-yaw adjustment system having an electromechanical device to which the camera can be attached, the pitch-yaw adjustment system designed to rotate the camera relative to the binocular viewing assembly around an axis parallel to the first axis and rotate the camera around an axis parallel to the second axis. In some embodiments, the control member is operatively coupled to the movement control system via sensors designed to detect movement of the control member, the sensors in communication with components of the movement control system In some embodiments, the control member can be operatively coupled to the movement control system via a gimbal having one or more sensors designed to detect movement of the control member, the sensors in communication with one or more components of the movement control system.

In some embodiments of the fourth aspect, the movement control system can be attached to the binocular viewing assembly. In some embodiments, the movement control system can be attached to an articulated arm. In some embodiments, the camera can be attached to the movement control system via an arm. In some embodiments, the medical apparatus can include a control system for controlling one or more electromechanical devices operatively coupled to the movement control system. In some embodiments, the control system can includes one or more pre-set positions for the movement control system

In a fifth aspect, a medical apparatus is provided that includes a display, a plurality of cameras and a processor, at least one of said cameras providing a surgical microscope view, said plurality of cameras comprising a first camera configured to image fluorescence in a surgical field and a second camera configured to produce a non-fluorescence image of said surgical field, a processor configured to receive video from said plurality of cameras and to display on said display a first fluorescence video from the first of said cameras and display a second non-fluorescence video from said second of said cameras.

In some embodiments of the fifth aspect, said first and second cameras have different spectral responses. In certain embodiments of the fifth aspect, one of the said first and second cameras is sensitive to infrared and the other is not.

In accordance with another aspect, a medical apparatus includes elements for stereo viewing positioned at or over a patient, the displays providing multiple views from various video sources within a surgical site and views from additional video sources above or obliquely viewing the surgical opening within a compact housing. The medical apparatus can include a switching module configured to switch between alternative sources of images, for example, different cameras on different surgical devices, such as cameras on retractors, cameras on surgical tools, a camera providing surgical microscope view, etc. and to present one or more of those images on one or more displays. Such images can be tiled, PIP, with certain images large and/or more central than others. Thumbnail images may also be included, such as for selection by a user.

Various embodiments enable viewing 3D or stereo images at, near, or over the patient with ergonomic characteristics for both the primary and assisting surgeon where a plurality of images from various sources may be viewed in real time with little or no latency.

Additionally, various embodiments provide a method for a surgical procedure using a stereo image acquisition system for viewing the beginning or entrance to the surgical site from above, horizontally or obliquely to the surgical site. Similar views can be provided at the close of the case when the surgeon(s) are closing the wound. Various embodiments are configured to accommodate the stereo image acquisition system that attaches mechanically and electrically to the ergonomic display. In some embodiments, the stereo image acquisition system is coupled to the ergonomic display so that the line of sight of the stereo image acquisition system is decoupled from the line of sight of oculars used to view the images acquired by the stereo image acquisition system. Without a line of sight requirement, the display system can retain a favorable ergonomic position and the stereo image acquisition system can be positioned independently.

Various embodiments comprise a method and system of stereo viewing positioned at or over the patient that displays multiple views from various sources within a surgical site and views from additional sources above or obliquely viewing the surgical opening within a compact housing.

Certain embodiments include the above system for a primary surgeon and an equivalent or similar system for an assistant surgeon. The two imaging systems allow the surgeons to be positioned within about 10 degrees of 180 degrees apart (e.g., across the table), or positioned within about 10 degrees of about 90 degrees apart. Other angular separations are also possible, such as any separation from about 20 degrees to about 180 degrees in either direction (e.g., positive or negative angles). This allows the pair of surgeons (e.g., primary and assistant) to be adjacent, shoulder-to-shoulder, across the table, or some other configuration. The positioning of the assistant surgeon can be accomplished without interrupting the primary surgeon by the use of a pivot component in the displays around which the assistant surgeon's display rotates. The dual displays attach to an arm and stand that can be positioned at, near, or over the patient.

Various embodiments include attachment points for a configurable stereo imaging acquisition assembly, which has 6 degree-of-freedom positioning, while maintaining the display systems in an ergonomic position favorable to both the primary and the assistant surgeon.

In various embodiments a display system comprises a first part comprising a binocular display assembly comprising an ergonomic binocular section containing an ocular, folding prism, and objective for each eye path. The second part comprises an electronic display assembly includes separate eye paths which are folded in a space saving manner, one or more electronic displays and optics that receive light from said one or more electronic displays and form a near constant diameter directed toward the side facing the surgeon. Such an optical layout may include small folding mirrors to keep the overall housing size compact enough to place over the patient and combine with a second stereo display system. The difference in distance between eye paths at a point in the system where the beams are collimated can be narrower than the inter-pupillary distance of the user. In this manner the overall size of the enclosure can be reduced or minimized.

In some embodiments, the optical paths for the left and right eyes can be separated at a collimated position, allowing the system to be made of first and second parts, the first part comprising an ergonomic binocular section containing an ocular, folding prism, and objective for each eye path. The second part includes an electronic display to be positioned at, over, or near the patient and includes separate eye paths which are folded in a space saving manner, and in particular maintain a near constant diameter from the side facing the surgeon towards the electronic display, with the last airspace between optics and display being a divergent path. Such an optical layout facilitates using small folding mirrors to keep the overall housing size compact enough to possibly place over the patient and combine with a second stereo display system. The eye path difference in distance at this collimated point in the system (where the two parts can separate) can be narrower than the inter-pupillary distance of the user. In this manner the overall size of the enclosure can be reduced or minimized.

In various aspects, a medical apparatus is provided. The medical apparatus can include a first display portion configured to display a first image and a second display portion configured to display a second image. The medical apparatus can also include electronics configured to receive one or more signals corresponding to images from a plurality of sources and to drive the first and second display portions to produce the first and second images based at least in part on the images from the plurality of sources. The medical apparatus can further include a first beam combiner configured to receive the first and second images from the first and second display portions and to combine the first and second images for viewing.

In certain embodiments, the first and second display portions can include first and second displays. The medical apparatus can further include imaging optics disposed to collect light from both the first and second display portions. The imaging optics can be configured to form images at infinity. The medical apparatus can further include a housing and a first ocular for viewing the combined first and second images within the housing. The medical apparatus can also further include a second ocular for viewing an additional image within the housing.

In some embodiments, the plurality of sources can include at least one camera providing a surgical microscope view. For example, the medical apparatus can further include the at least one camera providing the surgical microscope view. In some embodiments, the plurality of sources can include at least one camera disposed on a surgical tool. For example, the medical apparatus can further include the at least one camera disposed on the surgical tool. In some embodiments, the plurality of sources can include at least one source providing data, a computed tomography scan, a computer aided tomography scan, magnetic resonance imaging, an x-ray, or ultrasound imaging. For example, the medical apparatus can further include the at least one source providing the data, computed tomography scan, computer aided tomography scan, magnetic resonance imaging, x-ray, or ultrasound imaging. In various embodiments, the first image can include a fluorescence image and the second image can include a non-fluorescence image.

In various embodiments, the medical apparatus can further comprise a third display portion configured to display a third image and a fourth display portion configured to display a fourth image. The medical apparatus can further include a second beam combiner configured to receive the third and fourth images from the third and fourth display portions and to combine the third and fourth images for viewing. The third and fourth display portions can comprise third and fourth displays. The medical apparatus can further include additional electronics configured to receive one or more signals corresponding to images from another plurality of sources and to drive the third and fourth display portions to produce the third and fourth images based at least in part on the images from the another plurality of sources.

Some embodiments of the medical apparatus can further include imaging optics disposed to collect light from both the third and fourth display portions. The imaging optics can be configured to form images at infinity. The medical apparatus can further include a housing, a first ocular for viewing the combined first and second images within the housing, and a second ocular for viewing the combined third and fourth images within the housing.

In some embodiments, the another plurality of sources can include at least one camera providing a surgical microscope view. For example, the medical apparatus can further include the at least one camera providing the surgical microscope view. In some embodiments, the another plurality of sources can include at least one camera disposed on a surgical tool. For example, the medical apparatus can further include the at least one camera disposed on the surgical tool. In some embodiments, the another plurality of sources can include at least one source providing data, a computed tomography scan, a computer aided tomography scan, magnetic resonance imaging, an x-ray, or ultrasound imaging. For example, the medical apparatus can further include the at least one source providing the data, computed tomography scan, computer aided tomography scan, magnetic resonance imaging, x-ray, or ultrasound imaging. In various embodiments, the third image can include a fluorescence image and the fourth image can include a non-fluorescence image. In some embodiments, the medical apparatus can provide 3D viewing of a surgical field.

In certain embodiments of the medical apparatus, the combined first and second images for viewing can include a composite image of the first and second images. For example, the first beam combiner can be configured to produce the first image as a background image of the composite image, and to produce the second image as a picture-in-picture (PIP) of the composite image. Furthermore, in some embodiments, the combined third and fourth images for viewing can include a composite image of the third and fourth images. For example, the second beam combiner can be configured to produce the third image as a background image of the composite image, and to produce the fourth image as a picture-in-picture (PIP) of the composite image.

In various aspects, a binocular display for viewing a surgical field is provided. The binocular display can comprise one or more cameras configured to produce images of the surgical field, a left-eye view channel, and a right-eye view channel. The left-eye view channel can include a first display configured to display a left-eye view image of the surgical field and one or more first processing electronics. The right-eye view channel can include a second display configured to display a right-eye view image of the surgical field and one or more second processing electronics. Each of the first and second processing electronics can be configured to receive one or more user inputs, receive one or more input signals corresponding to the images from the one or more camera, select which image of the images from the one or more cameras to display, resize, rotate, or reposition the selected image based at least in part on the one or more user inputs, and produce one or more output signals to drive the first or second display to produce the left-eye or right-eye image. In some embodiments, each of the first and second processing electronics can include a microprocessor, a field programmable gate array (FPGA), or an application specific integrated circuit (ASIC).

In some embodiments of the binocular display, the one or more cameras can comprise at least one camera providing a surgical microscope view. In some embodiments, the one or more cameras can comprise at least one camera disposed on a surgical tool. In some embodiments, the one or more cameras can comprise a camera configured to produce a fluorescence image and a camera configured to produce a non-fluorescence image. In some embodiments, the binocular display can further include one or more sources providing data, a computed tomography scan, a computer aided tomography scan, magnetic resonance imaging, an x-ray, or ultrasound imaging. The binocular display can in some embodiments, provide 3D viewing of the surgical field.

Furthermore, in various embodiments of the binocular display, the one or more first processing electronics can include separate processing electronics for each of the one or more cameras configured to produce images on the first display. In some embodiments, the one or more second processing electronics can include separate processing electronics for each of the one or more cameras configured to produce images on the second display.

In accordance with another aspect, a medical apparatus is provided that includes a primary display housing, a display opening in the primary display housing, and one or more electronic displays disposed within the primary display housing, each of the one or more electronic displays comprising a plurality of pixels configured to produce a two-dimensional image. The medical apparatus also includes a display optical system disposed within the display housing, the display optical system comprising first imaging optics in a first optical path and second imaging optics in a second optical path. The medical apparatus also includes a binocular viewing assembly, a binocular opening in the binocular viewing assembly, and a binocular optical system comprising a first optical path to a first ocular in a pair of oculars and a second optical path to a second ocular in the pair of oculars. The first imaging optics is configured to receive a two-dimensional image from at least one of the one or more electronic displays and to produce a first collimated beam exiting the opening in the display housing, the second imaging optics is configured to receive a two-dimensional image from at least one of the one or more electronic displays and to produce a second collimated beam exiting the opening in the display housing, and the first and second collimated beams enter the binocular viewing assembly through the binocular opening and the first collimated beam is directed to the first optical path in the binocular optical system and the second collimated beam is directed to the second optical path in the binocular optical system.

In accordance with another aspect, a medical apparatus is provided that includes a primary display housing, a display opening in the primary display housing, and one or more electronic displays disposed within the primary display housing, each of the one or more electronic displays comprising a plurality of pixels configured to produce a two-dimensional image. The medical apparatus includes a display optical system disposed within the primary display housing, the display optical system comprising first imaging optics in a first optical path and second imaging optics in a second optical path. The medical apparatus also includes a binocular viewing assembly, a binocular opening in the binocular viewing assembly, and a binocular optical system comprising a first optical path to a first ocular in a pair of oculars and a second optical path to a second ocular in the pair of oculars, the first and second optical paths each comprising a redirection element. Each of the first and second imaging optics comprise a first lens element proximal to the one or more electronic displays and one or more redirection elements. The first lens element in each of the first and second imaging optics is smaller than each of the one or more electronic displays. The first and second collimated beams enter the binocular viewing assembly through the binocular opening and are directed to the pair of oculars.

In accordance with another aspect, a medical apparatus is provided that includes a viewing assembly comprising a housing, primary oculars, and assistant oculars, the assistant oculars configured to provide a view of an assistant electronic display disposed in the housing. The medical apparatus also includes an assistant optical assembly configured to provide an assistant surgical microscope view of the surgical site, the assistant optical assembly comprising an objective lens, an optical element configured to split light from the objective lens along at least two optical paths, a first optical path comprising first imaging optics and a second optical path comprising second imaging optics, a first assistant image sensor disposed at an image plane of the first optical path, and a second assistant image sensor disposed at an image plane of the second optical path, the second optical path orthogonal to the first optical path. The assistant electronic display is configured to display images based on images acquired by the first or second assistant image sensors.

In accordance with another aspect, a medical apparatus is provided that includes a viewing assembly comprising a housing, primary oculars, and assistant oculars, the primary oculars configured to provide a view of a primary electronic display disposed in the housing and the assistant oculars configured to provide a view of an assistant electronic display disposed in the housing. The medical apparatus also includes an optical assembly configured to provide a surgical microscope view of a surgical site. The optical assembly includes a primary image sensor at an image plane of a first optical path, an assistant image sensor at an image plane of a second optical path, and an objective lens configured to direct light to both the first and second optical paths. The primary and assistant electronic displays are configured to display images corresponding to the surgical microscope view of the surgical site.

In accordance with another aspect, a medical apparatus is provided that includes a viewing assembly comprising a housing, primary oculars, and assistant oculars, the primary oculars configured to provide a view of a primary electronic display disposed in the housing and the assistant oculars configured to provide a view of an assistant electronic display disposed in the housing. The medical apparatus also includes an optical assembly configured to provide a surgical microscope view of a surgical site, the optical assembly comprising a primary image sensor at an image plane of a first optical path and an assistant image sensor at an image plane of a second optical path. The medical apparatus includes an image processing system in communication with the assistant image sensor and the assistant electronic display, the image processing system comprising processing electronics. The image processing system is configured to receive video images acquired by the primary image sensor and the assistant image sensor, provide output primary video images based on the received video images from the primary image sensor, present the output primary video images on the primary display so that the output primary video images are viewable through the primary ocular, provide output assistant video images based on the received video images from the primary image sensor when the primary oculars and the assistant oculars have a relative orientation that is greater than about 170 degrees, wherein the output assistant video images are rotated 180 degrees relative to the output primary video imagers, and present the output assistant video images on the assistant display so that the output assistant video images are viewable through the assistant ocular.

In accordance with another aspect, a medical apparatus includes a viewing assembly comprising a housing, primary oculars, and assistant oculars, the primary oculars configured to provide a view of a primary electronic display disposed in the housing and the assistant oculars configured to provide a view of an assistant electronic display disposed in the housing. The medical apparatus includes an optical assembly disposed on the viewing assembly, the optical assembly configured to provide a surgical microscope view of a surgical site, the optical assembly comprising at least 4 auxiliary cameras, the at least 4 auxiliary cameras comprising a first stereo pair of cameras and a second stereo pair of cameras oriented orthogonally to the first stereo pair of cameras. The medical apparatus includes an image processing system in communication with the optical assembly, the primary electronic display, and the assistant electronic display. The image processing system includes processing electronics configured to receive primary video images acquired by the first stereo pair of cameras, provide primary output video images based on the received primary video images, and present the primary output video images on the primary electronic display so that the output video images are viewable through the primary oculars. The viewing assembly is configured to not provide a view of the surgical site via an optical path from the primary oculars or from the assistant oculars through an aperture in the housing.

In accordance with another aspect, a medical apparatus is provided that includes a primary viewing assembly comprising a primary housing and primary oculars configured to provide a view of a primary electronic display disposed in the primary housing. The medical apparatus also includes an assistant viewing assembly coupled to the primary viewing assembly, the assistant viewing assembly comprising an assistant housing and assistant oculars configured to provide a view of an assistant electronic display disposed in the primary housing. The medical apparatus includes an optical assembly disposed on the primary viewing assembly, the optical assembly configured to provide a surgical microscope view of a surgical site, the optical assembly comprising at least one auxiliary camera. The medical apparatus includes an image processing system in communication with the optical assembly, the primary electronic display, and the assistant electronic display. The image processing system includes processing electronics configured to receive video images acquired by the at least one auxiliary camera, provide output video images based on the received video images, and present the output video images on the primary electronic display so that the output video images are viewable through the primary oculars. The primary oculars and the assistant oculars can be moved independently of one another.

In accordance with another aspect, a medical apparatus is provided that includes a viewing assembly comprising a housing, primary oculars, and assistant oculars, the primary oculars configured to provide a view of a primary electronic display disposed in the housing and the assistant oculars configured to provide a view of an assistant electronic display disposed in the housing. The medical apparatus includes an optical assembly disposed on the viewing assembly, the optical assembly configured to provide a surgical microscope view of a surgical site, the optical assembly comprising at least one auxiliary camera. The medical apparatus includes one or more position sensors configured to determine a position of the assistant oculars relative to the primary oculars. The medical apparatus includes an image processing system in communication with the optical assembly, the primary electronic display, the assistant electronic display, and the position sensors. The image processing system includes processing electronics configured to receive video images acquired by the at least one auxiliary camera, provide primary output video images based on the received video images, present the primary output video images on the primary electronic display so that the primary output video images are viewable through the primary oculars, provide assistant output video images based on the received video images and the position of the assistant oculars relative to the primary oculars provided by the position sensors, and present the assistant output video images on the assistant electronic display so that the assistant output video images are viewable through the assistant oculars.

In accordance with another aspect, a medical apparatus is provided that includes a viewing assembly comprising a housing and primary oculars configured to provide a view of a primary electronic display disposed in the housing. The medical apparatus also includes a first camera configured to provide a surgical microscope view of a surgical site, a second camera configured to provide a view of the surgical site, the second camera positioned closer to the surgical site than the first camera, and a third camera configured to provide images from within the surgical site, the third camera positioned within an opening in a body created by an incision. The medical apparatus includes an image processing system in communication with the primary electronic display, the first camera, the second camera, and the third camera. The image processing system includes processing electronics configured to receive first video images acquired by the first camera, provide output first video images based on the received first video images, receive second video images acquired by the second camera, provide output second video images based on the received second video images, receive third video images acquired by the third camera, provide output third video images based on the received third video images, present the output first video images on the primary electronic display so that the output first video images are viewable through the primary oculars at an initial stage of a surgical procedure, present the output second video images on the primary electronic display so that the output second video images are viewable through the primary oculars when introducing a surgical tool into an opening of a body created by an incision during the surgical procedure, and present the output third video images on the primary electronic display so that the output third video images are viewable through the primary oculars when using the surgical tools in the opening of the body during surgical procedure.

In accordance with another aspect, a medical apparatus is provided that is configured to cleanse at least one camera disposed on a surgical device in a surgical site. The medical apparatus includes a housing comprising an elastic material configured to be disposed over the at least one camera. The medical apparatus includes one or more lines comprising an inlet and an outlet, the inlet configured to be connected to one or more fluid sources. The medical apparatus also includes one or more nozzles in fluid communication with the outlet, the one or more nozzles configured to deliver one or more hydraulic fluid from the one or more fluid sources to the at least one camera to remove obstructions from the at least one camera.

In various aspects, a medical apparatus including a retractor, a plurality of cameras, and a hydraulic system is provided. The retractor can be configured to hold open an incision and thereby provide a pathway for access of surgical tools to a surgical site. The plurality of cameras can be configured to acquire video images of the surgical site. At least some of the plurality of cameras can be disposed on the retractor and can be configured to acquire video images within the opening provided by the retractor. The hydraulic system can be configured to deliver pressurized fluid pulses to the plurality of cameras to remove obstructions therefrom while the cameras are disposed in the surgical site.

In certain embodiments, the cameras can comprise lenses or windows. The pressurized fluid pulses can be delivered to the lenses or windows for removing obstructions therefrom. The fluid pulses can comprise saline or physiological saline. In some embodiments, the medical apparatus can further comprise an image processor for processing input from the cameras and a display for displaying images from the cameras. The processor and display can be configured to provide a graphic user interface that enables control of the fluid delivery. For example, the graphic user interface can enable control of the frequency of the pressurized fluid pulses, the pressure of the pressurized fluid pulses, or both. In addition, some embodiments of the medical apparatus can further comprise a surgical tool having at least one of the plurality of cameras disposed thereon.

In various aspects, a medical apparatus can include a surgical device, at least one camera disposed on the surgical device, and a hydraulic system. The hydraulic system can be configured to deliver fluid to the at least one camera to remove obstructions therefrom. The hydraulic system further can be configured to deliver pressurized air to the at least one camera after the fluid is delivered. In various embodiments, the hydraulic system can be configured to deliver fluid pulses and air pulses to the at least one camera. In some such embodiments, a foot pedal can be configured to control actuation of the fluid and/or air pulses. For example, the foot pedal can comprise a proportional foot pedal.

Some embodiments of the medical apparatus can further include a processor and display configured to provide a graphic user interface that enables control of the fluid and air delivery. For example, the graphic user interface can enable control of the pressure of the air. In some such embodiments, the graphic user interface can enable control of the frequency of pressurized fluid pulse, the pressure of pressurized fluid pulses or both. In some embodiments, the surgical device can comprise a retractor or a surgical tool.

In further aspects, a medical apparatus can include a surgical device, at least one camera disposed on the surgical device, and a hydraulic system. The hydraulic system can be configured to deliver fluid to the at least one camera to remove obstructions therefrom. The hydraulic system can comprise a pulsing valve connected to a high pressure source of the fluid configured to provide pulses of fluid. In some examples, the pulsing valve can comprise a pop off valve configured to open when a pressure threshold is reached to provide increased pressure beyond the threshold value resulting in a pulse of liquid from the pulsing valve. In some examples, the at least one camera can comprise a plurality of cameras and the pulsing valve can be disposed in the hydraulic system such that the fluid is delivered to each of the plurality of cameras at the same time. For example, the pulsing valve can be disposed in a line that splits into different fluid outlets to clean different cameras. The pulsing valve ca be disposed upstream of the split. In certain embodiments of the medical apparatus, the hydraulic system can be further configured to deliver pressurized air to the at least one camera after the fluid is delivered. In some embodiments, the surgical device can comprise a retractor.

In various aspects, a medical apparatus can include a surgical device, at least one camera disposed on the surgical device, and a hydraulic system. The hydraulic system can be configured to deliver air to the at least one camera. The hydraulic system can comprise a pulsing valve connected to a high pressure source of air to provide pulses of air. The pulsing valve can comprise a pop off valve configured to open when a pressure threshold is reached to provide increased pressure beyond the threshold value resulting in a pulse of air from the pulsing valve.

In various aspects, a medical apparatus can include a surgical device, at least one camera disposed on the surgical device, and a hydraulic system. The at least one camera can have camera optics. The hydraulic system can be configured to deliver fluid and air to the camera optics of the at least one camera to remove obstructions therefrom. The hydraulic system can comprise a three way valve connected to a supply of the fluid and a supply of high pressure air. The three way valve can be configured to selectively shut off the supply of fluid and to provide instead pressurized air thereby reducing inadvertent leakage of fluid onto the camera optics. In some such embodiments, the hydraulic system can be configured to deliver fluid pulses and air pulses to the at least one camera. The medical apparatus can further comprise a pop off valve. The three way valve can be disposed downstream of the pop off valve. The surgical device can comprise a retractor.

In some aspects, a medical apparatus can include a surgical device, at least one camera disposed on the surgical device, and a hydraulic system. The at least one camera can have camera optics. The hydraulic system can comprise a valve connected to a high pressure source of fluid and configured to deliver fluid to the camera optics of the at least one camera to remove obstructions therefrom. The hydraulic system can be configured to open the valve periodically based on a pre-programmed schedule or a schedule selected by a user.

In further aspects, a medical apparatus can include a surgical device, at least one camera disposed on the surgical device, and a hydraulic system. The at least one camera can have camera optics. The hydraulic system can comprise a valve connected to a high pressure source of fluid and configured to deliver fluid to the camera optics of the at least one camera to remove obstructions therefrom. The hydraulic system can be configured to deliver fluid when an obstruction reducing the amount of light entering the camera is detected. For example, the at least one camera can produce an image signal and the apparatus can be configured to monitor the image signal to determine when visibility is compromised and thereby trigger delivery of the fluid to clean the camera optics. In some embodiments, the camera intensity can be monitored. For example, the attenuation of red wavelength compared to green wavelength can be monitored to determine whether blood is on the camera reducing the amount of light entering the camera.

In various aspects, a medical apparatus configured to clean at least one camera having a camera body disposed on a surgical device in a surgical site is provided. The medical apparatus can include a housing comprising an elastic material configured to be disposed over the at least one camera. The medical apparatus can also include one or more lines comprising an inlet and an outlet. The inlet can be configured to be connected to one or more fluid sources. The medical apparatus can further include one or more nozzles in fluid communication with the outlet. The one or more nozzles can be configured to deliver one or more hydraulic fluid from the one or more fluid sources to the at least one camera to remove obstructions from the at least one camera.

In some such embodiments, the housing can be elastic so as to be able to be stretched over and secure to the camera body. Also, at least one of the nozzles can be configured to deliver pressurized air to the at least one camera after pressurized saline is delivered. Some embodiments of the medical apparatus can further comprise a user interface configured to control delivery of the one or more hydraulic fluid.

In accordance with another aspect, a medical apparatus is provided that includes first and second electronic displays configured to produce two-dimensional images having parallax and two-dimensional images without parallax. The medical apparatus includes first and second imaging optics disposed respectively in first and second optical paths from the first and second electronic displays to form respective first and second collimated optical beams and images disposed at infinity. The medical apparatus includes a primary housing at least partially enclosing said displays and said imaging optics, and an opening in the housing. The first and second imaging optics are configured to direct said first and second beams through said opening such that a viewer, when viewing through a binocular assembly optically coupled to the opening, can see three-dimensional image content from the two-dimensional images having parallax and two-dimensional image content from the two-dimensional images without parallax. The three-dimensional image content is configured to be emphasized over said two-dimensional image content.

In various aspects, a medical apparatus can include first and second electronic displays, processing electronics, and a binocular viewer. The first and second electronic displays can be configured to produce two-dimensional images having parallax and two-dimensional images without parallax. When viewing through the binocular viewer, a viewer can see three-dimensional image content from the two-dimensional images having parallax and two-dimensional image content from the two-dimensional images without parallax. The three-dimensional image content can be configured to be emphasized over the two-dimensional image content. In some such embodiments, the three-dimensional image content can comprise brighter intensity than the two-dimensional image content. In other such embodiments, the three-dimensional image content can comprise higher saturation than the two-dimensional image content.

In accordance with another aspect, a medical apparatus is configured to calibrate a three-dimensional space of a surgical site being imaged. The medical apparatus includes imaging optics configured to project a calibration pattern onto said surgical site. The medical apparatus includes one or more cameras configured to image the projected calibration pattern and the surgical site. The medical apparatus includes processing electronics configured to determine information about said surgical site based on said image of said projected calibration pattern and said surgical site.

In further aspects, a medical apparatus can be configured to calibrate a three-dimensional space of a surgical site being imaged. The medical apparatus can include projection optics, one or more cameras, and processing electronics. The projection optics can be configured to project a calibration pattern onto the surgical site. The one or more cameras can be configured to image the projected calibration pattern and the surgical site. The processing electronics can be configured to determine information about the surgical site based on the image of the projected calibration pattern and the surgical site. In some examples, the processing electronics can be configured to generate a three-dimensional CAD rendition of the surgical site. The determined information can comprise at least one of depth information about the surgical site, distance between features in the surgical site, and volume information about the surgical site. The at least one of the one or more cameras can be configured to provide a surgical microscope view of the surgical site.

In accordance with another aspect, a medical apparatus is provided that includes a first display portion configured to display a first image and a second display portion configured to display a second image. The medical apparatus includes electronics configured to receive one or more signals corresponding to images from a plurality of sources and to drive the first and second display portions to produce the first and second images based at least in part on the images from the plurality of sources. The medical apparatus includes a first beam combiner configured to receive the first and second images from the first and second display portions and to combine the first and second images for viewing.

In accordance with another aspect, a surgical visualization system is provided that includes a plurality of cameras configured to acquire video images of a surgical site, the plurality of cameras comprising at least two cameras configured to acquire video images within the surgical site. The surgical visualization system also includes an actuator configured to be actuated by a user of the surgical visualization device to deliver one or more user interface signals, wherein the actuator is not configured to be actuated by a hand of the user. The surgical visualization system also includes an image processing system in communication with the plurality of cameras and the actuator. The image processing system includes processing electronics configured to receive the video images acquired by the plurality of cameras, provide a plurality of output video images, each of the plurality of output video images based on video images acquired by a corresponding one of the plurality of cameras, present one of the plurality of output video images on a display, and present a different one of the plurality of output video images on the display in response to a user interface signal received from the actuator.

In accordance with another aspect, a medical apparatus is provided that includes a display housing, an opening in the display housing, and an electronic display disposed within the display housing, the electronic display comprising a plurality of pixels configured to produce a two-dimensional image. The medical apparatus includes oculars configured to provide a view of the display within the display housing. The medical apparatus includes an imaging system disposed on the display housing, the imaging system configured to generate images of a surgical site from outside the surgical site using a right eye camera and a left eye camera configured respectively to produce a right eye video stream of the surgical site to produce a left eye video stream of the surgical site. The imaging system includes a common objective for the left eye optical path and the right eye optical path, the common objective configured to collimate light from the surgical site, right eye optics configured to form an image at a right eye image plane, left eye optics configured to form an image at a left eye image plane, a right eye camera configured to generate a video stream based on the image at the right eye image plane, and the left eye camera configured to generate a video stream based on the image at the left eye image plane.

In accordance with another aspect, a medical apparatus can comprise a surgical device configured to be powered by fluid; and a fluidic system configured to provide fluidic power to the surgical device, the fluidic system comprising a fluidic turbine operably connected to the surgical device to actuate the surgical device. In some embodiments, the fluidic turbine can be configured to be powered by hydraulic fluid. In some embodiments, the medical apparatus can further comprise a hydraulic fluid source in fluid communication with the surgical device. In some embodiments, the fluidic turbine can be configured to be powered by pneumatic fluid. In some embodiments, the medical apparatus can further comprise a pneumatic fluid source in fluid communication with the surgical device In some embodiments, the fluidic turbine can be configured to be powered by a mixture of hydraulic fluid and pneumatic fluid. In some embodiments, the medical apparatus can further comprise a control configured to allow a user to adjust an amount of hydraulic fluid and an amount of pneumatic fluid delivered to the fluidic turbine. In some embodiments, the surgical device can be a drill.

In accordance with another aspect, a surgical tool can comprise a proximal actuation element; a piston operably connected to the proximal element and disposed distal to the proximal actuation element; and a distal actuation chamber disposed distal to the piston, wherein the distal actuation chamber is configured to receive fluid via a return valve, thereby moving the piston in a proximal direction and compressing the proximal actuation element. In some embodiments, the surgical tool is a Kerrison. In some embodiments, the distal actuation chamber is configured to receive physiological saline. In some embodiments, the distal actuation chamber is configured to receive air or gas. In some embodiments, the surgical tool further comprises a biasing member (e.g., a spring) disposed in the distal actuation chamber. In other embodiments, the biasing member can be excluded.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 illustrates an embodiment of the surgical visualization system having an imaging system that can be configured to provide imagery similar to a direct-view surgery microscope.

FIGS. 2A-C show one embodiment a surgical retractor device that includes an integrated imaging assembly.

FIG. 3 illustrates an example surgical viewing system attached to an articulating arm, the system including one or more cameras mounted on a binocular viewing platform.

FIGS. 4A and 4B illustrate an example surgical viewing system that includes an isocenter positioning system attached to the binocular viewing platform.

FIGS. 5A and 5B illustrate an embodiment of a surgical visualization system having an optical imaging system mounted under the binocular viewing platform.

FIGS. 6A-6E illustrate embodiments of optical imaging systems for use in a stereoscopic surgical viewing system, such as those illustrated in FIGS. 5A and 5B.

FIG. 7A is a front view of an embodiment of a surgical visualization system, a movement control system, and an imager.

FIG. 7B is a front view of the embodiment of FIG. 7A with the movement control system and imager shifted.

FIG. 7C is a partial section view of the embodiment of a movement control system of FIG. 7A.

FIG. 8 is a side view of an embodiment of a surgical visualization system, a movement control system, and an imager.

FIG. 9 is a rear view of an embodiment of an embodiment of a movement control system.

FIGS. 10A-10D illustrate example display optical systems configured to provide a view of a display or a pair of displays through oculars.

FIGS. 11A-11G illustrate example display optical systems configured to deliver to oculars images of a display wherein light paths that would intersect a viewing assembly are reduced or eliminated through baffles.

FIG. 12 illustrates a front view of an example binocular assembly and display enclosure assembly.

FIGS. 13A and 13B illustrate example binocular assemblies with primary and assistant binoculars.

FIGS. 14 and 15 illustrate collimated eye paths that have a near constant diameter through a series of fold mirrors.

FIG. 16 illustrates the center-to-center distance of the displays being greater than the inter-pupillary distance of the surgeon.

FIG. 17 illustrates the two eye path pupils and their respective center lines passing from a common mounting face that encircles both eye paths.

FIG. 18 illustrates the first two turning mirrors, which adjust for the difference between the center-to-center distance of the displays and the inter-pupillary distance of the user in order to produce a compact electronic display with compact dimensions in axial and width dimensions for surgical imaging in 3D, stereo.

FIG. 19 illustrates a camera or stereo camera pair is disposed on the binocular display assembly configured to provide isocentered motion.

FIG. 20 illustrates tilting a camera or stereo camera pair in an orthogonal direction configured to provide isocentered motion.

FIG. 21 illustrates surgical microscope camera views from a temporal direction provided using a surgical microscope camera.

FIG. 22 illustrates how the assistant opposite of the surgeon (with the assistant display assembly directed 180° with respect to the surgeon display assembly) is to see the images reoriented (e.g., upside down) and with the locations of the images reversed.

FIG. 23 illustrates how four cameras nested in a 2×2 array can provide for surgeon view as well as assistant view where assistant is on the right side of the surgeon viewing form an orthogonal direction (e.g., 90°).

FIG. 24 illustrates a system for providing multiple cameras providing surgical microscope views for the assistant in addition to primary surgeon view.

FIG. 25 is a schematic illustration of a surgical visualization system with an assistant display and a panel display viewable by an assistant.

FIG. 26 illustrates an example imaging system comprising an objective triplet.

FIG. 27 illustrates an example imaging system comprising a common objective lens for both optical paths of stereo imagers.

FIG. 28 illustrates a zoom lens group and a video coupler optical system of the example imaging system.

FIGS. 29A-29B illustrate respective side and top views of the example imaging system having a common objective lens for stereo imagers.

FIG. 30 illustrates an example stereo imaging assembly.

FIG. 31 schematically illustrates an example medical apparatus in accordance with certain embodiments described herein.

FIG. 32A-32C schematically illustrate another example medical apparatus in accordance with certain embodiments described herein.

FIG. 33A illustrates a schematic of an example of a composite image with a picture-in-picture (PIP) view of a surgical field.

FIG. 33B schematically illustrates a front view of an embodiment of a medical apparatus incorporating left and right assemblies to produce a composite image of two or more images for both left and right eyes.

FIG. 33C illustrates a schematic of an example view of multiple images of a surgical field combined adjacent to one another.

FIGS. 34A-C illustrate an irrigation assembly for cleansing an optical sensor.

FIG. 35 illustrates an example camera supported on a platform configured to attach to a retractor having an elastic cover over the camera that includes hydraulic and/or pneumatic pathways for cleaning camera optics.

FIG. 36 illustrates a housing for the camera fluidics used for cleaning the camera that is disposed on the camera optics.

FIG. 37 illustrate a side cross sectional view of one embodiment of fluidic pop off valves that may be encased in an elastic housing or bladder that may be configured to be slipped onto a platform as described above with respect to FIGS. 35 and 36.

FIG. 38 illustrates an example hydraulic line that provides hydraulics for cleaning camera optics.

FIGS. 39A-C illustrate an embodiment of a Kerrison that can be operated hydraulically and/or pneumatically.

FIG. 40A shows a perspective cross-section of a hydraulic turbine.

FIG. 40B shows a cross-section of a portion of the hydraulic turbine of FIG. 40A.

FIG. 40C shows a cross-section of a portion of the hydraulic turbine of FIG. 40A and a diverted fluid flow path.

FIG. 41 shows one embodiment of an impeller.

FIG. 42 is a schematic representation of an embodiment of a turbine.

FIG. 43 is a graphical representation of a mathematical relationship between exit stream velocity and centrifugal acceleration of the turbine of FIG. 42.

### DETAILED DESCRIPTION

The following description is directed to certain embodiments for the purposes of describing the innovative aspects of this disclosure. However, a person having ordinary skill in the art will readily recognize that the teachings herein can be applied in a multitude of different ways. The described embodiments may be implemented in any device or system that can be configured to provide visualization of a surgical site. Thus, the teachings are not intended to be limited to the embodiments depicted solely in the figures and described herein, but instead have wide applicability as will be readily apparent to one having ordinary skill in the art.

### Surgical Visualization System

To provide improved visualization of a surgical site, a surgical device can be provided with multiple integrated cameras. Each of the cameras may capture a distinct view of the surgical site. In some embodiments, imagery from the plurality of cameras may be displayed to facilitate operation in a surgical site. Tiled, individual, and/or stitched imagery from the multiple cameras can provide the user with a view of the surgical site. The user can select the imagery to be displayed and the manner in which it is displayed for enhanced utility during surgery. As used herein, the term imagery and images includes video and/or images captured from one or more video cameras. Images from video are often referred to as video images or simply images. The term images may also refer to still images or snap shots. Video feed or video stream may also be used to describe the video images such as video images from a camera.

The video cameras may comprise, for example, CCD or CMOS sensor arrays or other types of detector arrays. A frame grabber may be configured to capture data from the cameras. For example, the frame grabber may be a Matrox Solios eA/XA, 4 input analog frame grabber board. Image processing of the captured video may be undertaken. Such image processing can be performed by, for example, the Matrox Supersight E2 with Matrox Supersight SHB-5520 with two Intel Six Core Xeon E5645 2.4 GHz processors with DDR3-1333SDRAM. This system can be designed to support eight or more camera inputs using two Matrox Solios eA/XA, 4 input, analog frame grabber boards. More or less cameras may be employed. In some implementations, a field programmable gate array (“FPGA”) can be used to capture and/or process video received from the cameras. For example, the image processing can be performed by Xilinx series 7 FPGA boards. Other hardware devices can be used as well, including ASIC, DSP, computer processors, a graphics board, and the like. The hardware devices can be standalone devices or they can be expansion cards integrated into a computing system through a local computer bus, e.g., a PCI card or PCIe card.

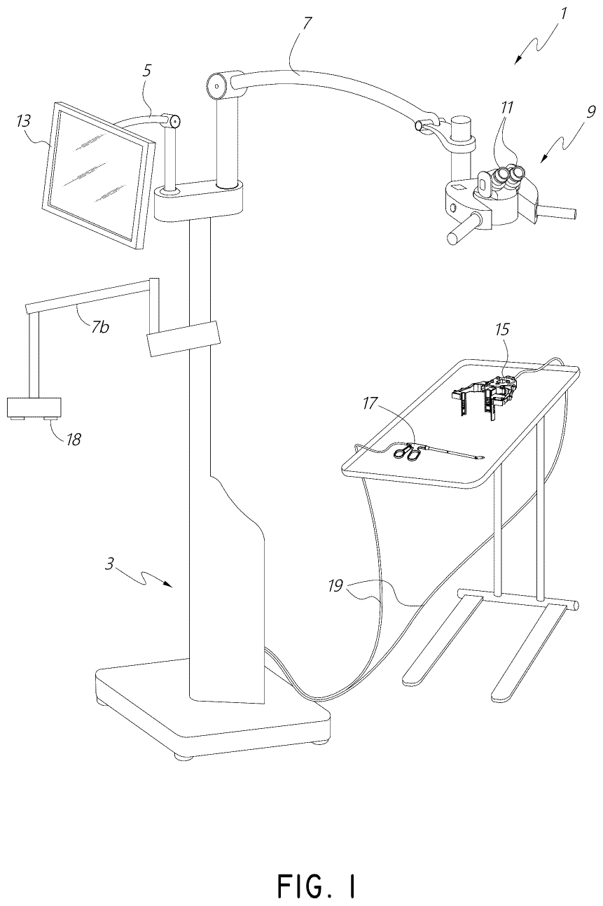

FIG. 1 shows an example embodiment of a surgical visualization system 1. As illustrated, the system 1 includes a console and electronics 3 from which three arms 5, 7 and 7b extend. The first arm 5 has mounted to its distal end a viewing platform 9. The viewing platform may include two oculars 11 and be configured similarly to a standard surgical microscope viewing platform. In some embodiments, however, unlike a conventional surgical microscope or a head mounted display the viewing platform 9 is not a direct view device where the surgeon or other user sees directly through the platform, e.g., an aperture in the platform. In some embodiments, regardless whether the user can view directly through the viewing platform, the surgical visualization system 1 can be configured to display video in a manner that the video displayed is decoupled from movement of the surgical microscope cameras such that a user can adjust the position and/or orientation of the surgical microscope cameras without moving the oculars 11 or the user adjusting position. As discussed in more detail below, the viewing platform 9 may include displays that receive signals from cameras that the surgeon or user employs to view the surgical site.

In some embodiments, cameras can be mounted to the viewing platform 9 and the cameras can be configured to provide imagery of the surgical site. Accordingly, the cameras can be used to provide imagery similar to a conventional surgical microscope. For example, the cameras on the viewing platform 9 can be configured to provide a working distance, or a distance from the viewing platform 9 to the patient, that can vary using zooming. The virtual working distance can vary, where the working distance can be at least about 150 mm and/or less than or equal to about 450 mm, at least about 200 mm and/or less than or equal to about 400 mm, or at least about 250 mm and/or less than or equal to about 350 mm. The working distance can be selected and/or changed by the surgeon. In some embodiments, changing the working distance does not affect the position and/or orientation of the oculars 11 with respect to the user or surgeon. In some embodiments, the cameras mounted on the viewing platform 9 can be used to provide gesture recognition to allow a surgeon to virtually interact with imagery provided by the display using the surgeon's hands, a surgical tool, or both, as described in greater detail herein.

The second arm 5 has mounted to its distal end an input and display device 13. In some embodiments, the input and display device 13 comprises a touchscreen display having various menu and control options available to a user. In some embodiments, the touchscreen can be configured to receive multi-touch input from ten fingers simultaneously, allowing for a user to interact with virtual objects on the display. For example, an operator may use the input device 13 to adjust various aspects of the displayed image. In various embodiments, the surgeon display incorporating a video camera providing a surgical microscope view may be mounted on a free standing arm, from the ceiling, on a post, or the like. The flat panel display touch screen 13 may be positioned on a tilt/rotate device on top of the electronics console.

A surgical tool 17 can be connected to the console 3 by electrical cable 19. The surgical tool 17 includes, for example, a cutting tool, a cleaning tool, a device used to cut patients, or other such devices. In other embodiments, the surgical tool 17 may be in wireless communication with the console 3, for example via WiFi (e.g., IEEE 802.11a/b/g/n), Bluetooth, NFC, WiGig (e.g., IEEE 802.11ad), etc. The surgical tool 17 may include one or more cameras configured to provide imagery, e.g., image and/or video data. In various embodiments, video data can be transmitted to a video switcher, camera control unit (CCU), video processor, or image processing module positioned, for example, within the console 3. The video switching module may then output a display video to the viewing platform 9. The operator may then view the displayed video through the oculars 11 of the viewing platform 9. In some embodiments, the binoculars permit 3D viewing of the displayed video. As discussed in more detail below, the displayed video viewed through the viewing platform 9 may comprise a composite video formed (e.g., stitched or tiled) from two or more of the cameras on the surgical tool 17. Cameras of certain embodiments can be disposed on a surgical tool. In various embodiments, cameras can be disposed on a retractor 15 configured to hold open a surgical incision and to provide access to the surgical site.

In use, an operator may use the surgical tool 17 to perform open and/or minimally invasive surgery. The operator may view the surgical site by virtue of the displayed video in the viewing platform 9. Accordingly, the viewing platform (surgeon display system) 9 may be used in a manner similar to a standard surgical microscope although, as discussed above, the viewing platform 9 need not be a direct view device wherein the user sees directly through the platform 9 to the surgical site via an optical path from the ocular through an aperture at the bottom of the viewing platform 9. Rather in various embodiments, the viewing platform 9 includes a plurality of displays, such as liquid crystal or light emitting diode displays (e.g., LCD, AMLCD, LED, OLED, etc.) that form an image visible to the user by peering into the ocular. Accordingly, one difference, however, is that the viewing platform 9 itself need not necessarily include a microscope objective or a detector or other image-capturing mechanisms. Rather, the image data can be acquired via the cameras of the surgical tool 17. The image data can then be processed by a camera control unit, video processor, video switcher or image processor within the console 3 and displayed imagery may then be viewable by the operator at the viewing platform 9 via the display devices, e.g., liquid crystal or LED displays, contained therein. In some embodiments, the viewing platform 9 can provide a view similar to a standard surgical microscope using cameras and displays and can be used in addition to or in conjunction with a standard surgical microscope optical pathway in the viewing platform. In certain embodiments, the viewing platform 9 can provide a surgical microscope view wherein changes in the viewing angle, viewing distance, work distance, zoom setting, focal setting, or the like is decoupled from movement of the viewing platform 9. In certain embodiments, changes in the position, pitch, yaw, and/or roll of the imaging system 18 are decoupled from the viewing platform 9 such that the imaging system 18 can move and/or re-orient while the surgeon can remain stationary while viewing video through the oculars 11.

The third arm 7b can include an imaging system 18 that can be configured to provide video similar to a direct-view surgery microscope. The imaging system 18 can be configured, then, to provide a surgical imaging system configured to provide an electronic microscope-like view that can comprise video of the work site or operational site from a position above the site (e.g., about 15-45 cm above the surgical site) or from another desired angle. By decoupling the imagers 18 from the display, the surgeon can manipulate the surgical imaging system to provide a desired or selected viewpoint without having to adjust the viewing oculars. This can advantageously provide an increased level of comfort, capability, and consistency to the surgeon compared to traditional direct-view operating microscope systems. In some embodiments, as described herein, the imagers 18 can be located on one or more of the following: on a viewing arm, the viewing platform 9, on a dedicated arm 7b, on a display arm 5, on a separate post, a separate stand, supported from an overhead structure, supported from the ceiling or wall, or detached from other systems. For example, a camera or imager providing a surgical microscope view can be on dedicated arm 7b or a separate post or stand or support and not on the viewing platform 9. The imagers 18 can comprise a camera configured to be adjustable to provide varying levels of magnification, viewing angles, monocular or stereo imagery, convergence angles, working distance, or any combination of these.

The viewing platform 9 can be equipped with wide field-of-view oculars 11 that are adjustable for refractive error and presbyopia. In some embodiments, the oculars 11, or eyepieces, may additionally include polarizers in order to provide for stereoscopic vision. The viewing platform 9 can be supported by the arm 7 or 7b, such that it may be positioned for the user to comfortably view the display 13 through the oculars 11 while in position to perform surgery. For example, the user can pivot and move the arm 7 or 7b to re-orient and/or re-position the viewing platform 9.

In some embodiments, the image processing system and the display system are configured to display imagery placed roughly at infinity to reduce or eliminate accommodation and/or convergence when viewing the display. In some embodiments, a surgical retractor can be included that is configured to hold open an incision and thereby provide a pathway for access of surgical tools to a surgical site, said retractor comprising portions configured to be disposed about an open central region centrally located between said retractor portions so as to permit access of surgical tools to the surgical site through said open central region. In various embodiments, the retractor can include at least two cameras directed inward toward the central open region and at least one of the at least two cameras directed downward into the surgical field. A display optical system can include one or more lenses and one or more redirection elements (e.g., mirrors, prisms) and can be configured to provide light from the display that can be imaged by a binocular viewing assembly comprising a pair of oculars, objectives, and/or turning prisms or mirrors. The display devices such as liquid crystal displays can be imaged with the objective and the pair of oculars and display optical system within the viewing platform 9. The binocular assembly and display optical system can be configured to produce an image of the displays at infinity. Such arrangements may potentially reduce the amount of accommodation by the surgeon. The oculars can also have adjustments (e.g., of focus or power) to address myopia, hyperopia, and/or presbyopia of the surgeon. For example, each ocular can have a variable adjustable power to provide optical correction that the surgeon or other user may desire. Accordingly, the oculars may provide optical correction allowing the surgeon or other users to view the displays through the oculars without wearing glasses even if ordinarily prescription glasses were worn for other activities.

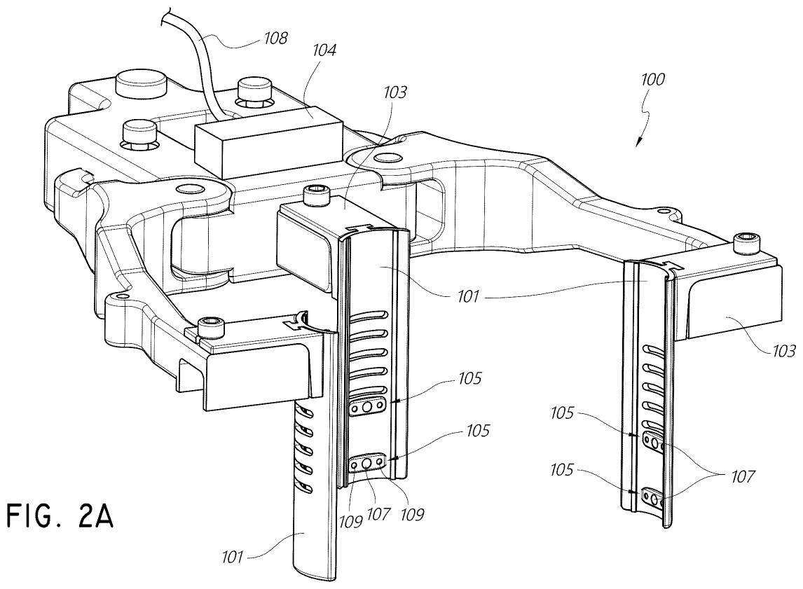

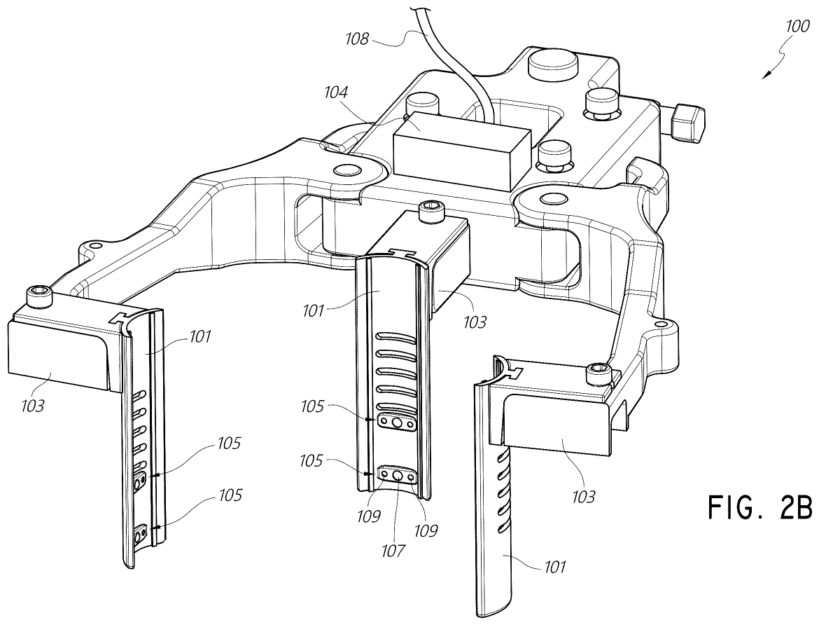

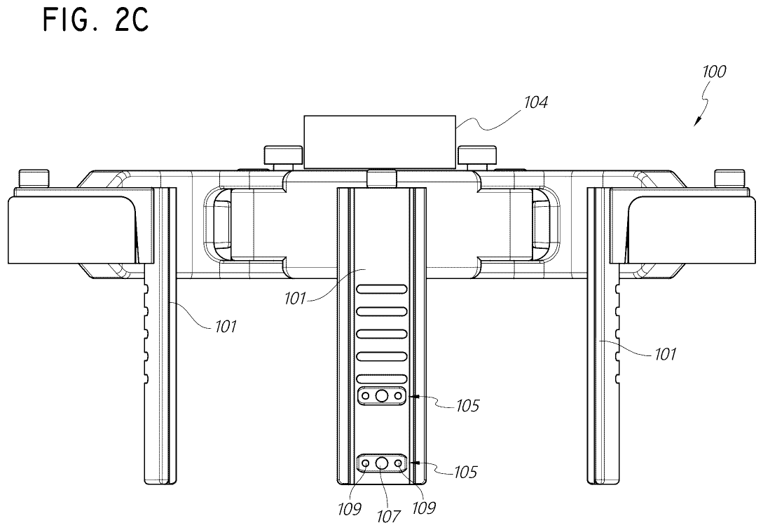

FIGS. 2A-C show one embodiment a surgical retractor device that includes an integrated imaging assembly. In some embodiments, the imaging assembly includes a plurality of integrated cameras. The retractor 100 includes three blades 101, however, more or less may be included depending on the design. Each of the blades may be attached to an articulable arm 103 that allows for the position of the blades to be adjusted during the operation. For example, following a small incision, the three blades 101 can be arranged in a closed position where each are positioned close to one another. In this closed configuration, the three blades can be introduced through the incision, and then expanded to provide for an operating pathway or working space. In other embodiments 4, 5, 6, 7, 8 or more blades, fingers, retractor members, or other barriers may be employed (or fewer members such as two blades, etc., or even a single member such as a single lumen of a tubular retractor may be used). In various embodiments, the surgical area may be at least 400 mm², for example, have an opening with an areas between 400 and 2100 mm². The working space may be an area centrally located between retractor blades (or within the lumen of a tubular retractor) that allows for surgical tools or other instruments to pass through. As shown, the retractor does not obstruct the center of the retractor (e.g., array of retractor blades, finger, members, etc., or lumen of a tubular retractor) and the open region formed by the retractor and permits unobstructed access to the center of the surgical site for ready access by the surgeon. Each of the blades 101 includes one or more integrated cameras, or cameras with combined stereo paths to one sensor or camera module 105. In various embodiments the number of camera modules and configurations can vary. In the illustrated embodiment, each camera module 105 includes a camera 107 and one or more, or two illumination sources 109 disposed on opposite sides of the camera 107. In various embodiments, the number of illumination sources per camera module may vary. In some embodiments, the illumination sources may not be disposed directly adjacent any particular camera. In some embodiments, the illumination sources can be omitted, and the camera module can rely on ambient supplementary or overhead light or light directed from a light source located elsewhere. In some embodiments, the orientation of an integrated camera 107 may be substantially fixed with respect to the retractor blade 101 or other surgical tool. In some embodiments, the camera 107 and/or the camera module 105 may be adjustable with respect to the retractor blade 101.

In the illustrated embodiment, the retractor blades 101 are substantially rigid. In various embodiments, the retractor blades may be malleable, and may have a wide range of different structural features such as width, tension, etc. For example, stronger, larger retractor blades may be desired for spinal and trans-oral surgery, while weaker, smaller retractor blades may be desired for neurosurgery. In some embodiments, the retractor can be configured such that different blades can be arranged as desired.

Each of the camera modules 105 are in electrical communication with an aggregator 104. The aggregator 104 is configured to receive input from each of the camera modules 105, and to connect to external components via electrical cable 108. For example, the hub or aggregator 104 may receive image data from each of the camera modules 105 and may transmit the image data to an image processing module (not shown). In the illustrated embodiment, the wiring connecting the camera modules 105 with the aggregator 104 is imbedded within the retractor blades 101 and articulating arms 103 and is not visible. In some embodiments, as described in more detail below, cables connecting the camera modules 105 with the aggregator 104 may be adhered (either permanently or non-permanently, e.g., releasably) to the exterior surface of the retractor 100. In the illustrated embodiment, the hub or aggregator 104 is affixed to an upper surface of the retractor 100. The aggregator may be positioned at any locations relative to the retractor 100, or may be disconnected from the retractor 100 altogether. The aggregator may contain camera interface electronics, tracker interface electronics and SERDES to produce a high speed serial cable supporting all cameras in use. In various embodiments, the retractor camera output is coupled to a console which causes video from the retractor camera to be presented on display.

Although the illustrated embodiment shows integrated camera modules 105, in various embodiments the camera modules 105 may be removably attached to the retractor blades 101. In some embodiments, the camera modules 105 can be disposed within pre-positioned receptacles on the retractor blades 101 or other surgical device. In some embodiments, the camera modules 105 can be disposed at a plurality or range of locations desired by the user on the retractor blades 101. In various embodiments, the orientation and position of the sensors can be adjusted by the user, e.g., physician, nurse, technician, or other clinician. In some embodiments, for example, the camera may be disposed on a track such that the camera can slide up and down the retractor, e.g., retractor blade. The height of the camera or camera within or above the surgical site may thereby be adjusted as desired. Other arrangements for laterally adjusting the position of the camera may be used. Additionally, in various embodiments, the cameras may be configured to have tip and/or tilt adjustment such that the attitude or orientation of the camera may be adjusted. The line of sight or optical axis of the cameras can thereby be adjusted to, for example, be directed more downward into the surgical site or be directed less into the surgical sight and more level or angled in different lateral directions. The camera modules 105 can include sensors or markers for, e.g., electromagnetic or optical tracking or use encoders, accelerometers, gyroscopes, or inertial measurement units (IMUs) or combinations thereof or any other orientation and/or position sensors, as described in more detail below. Tracking can provide location and/or orientation of the cameras. The images obtained by the cameras may be stitched together or tiled using image processing techniques. Tracking or otherwise knowing the relative locations of the sensor can assist in image processing and display formatting.

In various embodiments, pairs of cameras together provide information for creating a stereo effect or 3-dimensional (3D) image. Pairs of cameras, for example, may be included on each of the blades 101 of the retractor 100.

As illustrated, the retractor is configured to hold open tissue so as to produce an open region or cavity centrally located between the blades. Notably, in various embodiments, this open central region is unobstructed by the retractor. In particular, the central portions of the open region would be unobstructed by features of the retractor such that the surgeon would have clear access to the surgical site. The surgeon could thus more freely introduce and utilize his or her tools on locations within the surgical site. Additionally, this may enable the surgeon to use tools with both hands without the need to hold an endoscope.

Also as illustrated, the cameras are disposed on the blades of the retractor such that the cameras face inward toward with respect to each other (and possibly downward and/or into the surgical site), the opening or surgical site held open by the retractor blades. The cameras in this example would be disposed about the central open region held open by the retractor blades so as to provide views from locations surrounding the surgical site. The camera thus would face objects within the surgical site such as structures on which tools would be used by the surgeon to operate, as stated above possibly downward and/or into the surgical site. Accordingly, at least two cameras directed inward toward a central open region of the surgical site (e.g., an opening created by an incision and/or held open by a retractor) and at least one of the at least two cameras can be directed downward into the surgical site or field.

In this particular example, the cameras on two of the blades face each other such that the leftmost blade and the cameras thereon would be in the field-of-view of the cameras on the rightmost blade and vice versa. The cameras on the leftmost blade may be anti-parallel to the cameras on the rightmost blade and have optical axes oriented at an angle, θ, of 180° with respect to each other. The cameras on the remaining blade may be directed orthogonally to the other two blades and thus have optical axes directed at an angle, θ, of 90° with respect to each other. Retractors with cameras can be reaffixed to a frame or mounting structure during a procedure and the cameras can reorient themselves with respect to relative position within an array of the cameras through their communication protocol with the aggregator and video switching unit.

In some embodiments, the field-of-views of the different cameras, and hence the images produced by the different cameras, may overlap. Image processing may be employed to yield increased resolution at the regions of overlap. Likewise, the number of sensors used may be increased to provide increased field-of-view and/or resolution. Likewise, cameras with overlapping images can be electronically magnified thereby making their images adjacent rather than overlapping.

A minimally invasive spine surgery can use a tubular retractor having a circular working space with a diameter of approximately 25 mm. The retractor contains blades, fingers, or at least one barrier such as e.g., a tube that holds tissue back to maintain open the surgical site. Multiple cameras located on the retractor at locations within the surgical field or in very close proximity thereto, e.g., within 75 mm of the surgical opening, can provide a useful viewpoint for the surgeon. The cameras may for example be located on the blades, fingers, tubular barrier, or other portion of the retractor close to the surgical field or within the patient and the surgical field. The cameras may include pairs of cameras arranged and/or oriented to provide stereo and thus 3D imaging or single CMOS camera chips with dual optics to provide stereo. The cameras may be located at various locations in relation to surgical devices, for example, the cameras can be located proximally and distally along or near a retractor, wherein the location of the cameras can be configured to facilitate both the progression of surgery and an enhanced view or view selection of an area of interest. The cameras can be faced downward into the surgical site and inward toward each other. The retractor can be used to hold open an incision to provide access to a surgical site.

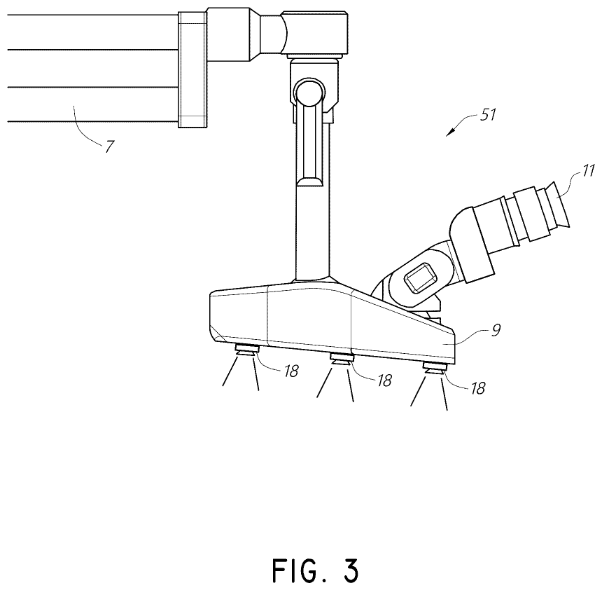

In some embodiments, the viewing platform 9 can include one or more imagers configured to provide electronic microscope-like imaging capabilities. FIG. 3 illustrates an example surgical imaging system 51 attached to an arm 7, the system 51 including one or more cameras 18 mounted on a viewing platform 9. The cameras 18 can be configured to provide imagery of a worksite. The image data can be presented on a display that the user can view using oculars 11 mounted on the viewing platform 9. This design can be used to mimic other direct-view microscopes, but it can also be configured to provide additional capabilities. For example, the surgical imaging system 51 can be configured to have a variable working distance without adjusting the viewing platform 9 or the articulating arm 7. The surgical imaging system 51 can be configured to provide image processing capabilities such as electronic zooming and/or magnification, image rotation, image enhancement, stereoscopic imagery, and the like. Furthermore, the imagery from the cameras 18 can be combined with imagery from cameras on the surgical device 17. In some embodiments, the surgical imaging system 51 can provide fluorescence images.

Although the discussion considers images from surgical tools, numerous embodiments may involve at least one auxiliary video camera 18 and one or more other cameras that are not disposed on surgical tools but are disposed on other medical devices. These medical devices may include devices introduced into the body such as endoscopes, laparoscopes, arthroscopes, etc.

Accordingly, one or more displays such as the at least one display 13 included in the viewing platform 9 may be used to provide a surgical microscope view using one or more cameras such as the auxiliary video camera(s) 18 as well as to display views from one or more cameras located on such medical devices other than surgical tools. As illustrated, a surgical microscope camera can be on a different platform other than the viewing platform 9 and includes features described herein with respect to cameras or imagers on the viewing platform, including but not limited to, isocentered motion, variable work distance, and movement control system. A switching module can be included to switch between views or combinations of views. In some embodiments, cameras from a variety of sources, e.g., surgical tools and other medical devices, in any combination, may be viewed on the display(s) on the surgical platform together with the surgical microscope view from the auxiliary video cameras 18. As described herein, the displays may provide 3D thus any of the images and graphics may be provided in 3D.

In various embodiments, a virtual touchscreen may be provided by the auxiliary video cameras 18 or other virtual touchscreen cameras mounted to the viewing platform 9. Accordingly, in some embodiments a user may provide a gesture in the field of view of the auxiliary video cameras and/or virtual touchscreen cameras and the processing module can be configured to recognize the gesture as an input. Although the virtual display has been described in the context of the auxiliary video cameras 18, other cameras, e.g., virtual reality input cameras, possibly in addition to the auxiliary video cameras 18 may be used. These cameras may be disposed on the viewing platform 9 or elsewhere, such as the third arm 7b. As described herein the displays may provide 3D thus the virtual reality interface may appear in 3D. This may increase the immersive quality of the viewing experience, enhancing the detail and/or realistic presentation of video information on the display.

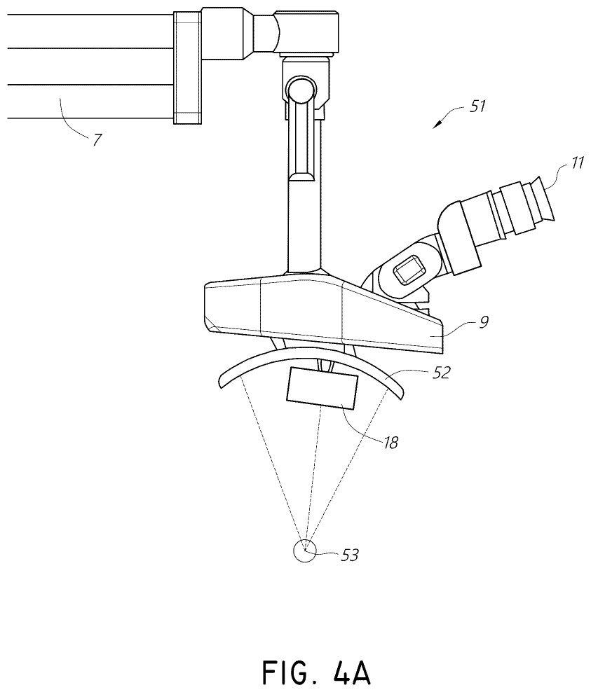

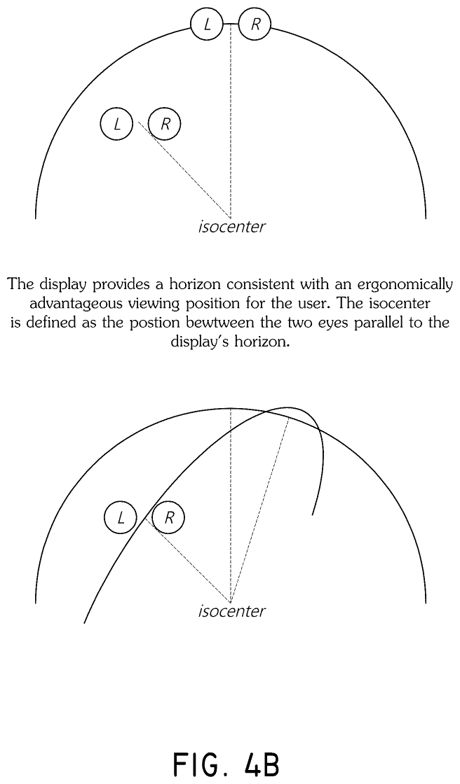

In some embodiments, as illustrated in FIG. 4A, the surgical imaging system 51 includes an isocenter positioning system 52 attached to the viewing platform 9. The isocenter positioning system 52 can include a single track or guide configured to move and orient the cameras 18 such that they are substantially pointed at a single point 53, the isocenter. In some embodiments, a second track or guide can be attached to the first guide in an orthogonal manner to provide movement along two dimensions while substantially maintaining the pointing angle towards the isocenter 53. Other configurations can be used to provide isocenter pointing capabilities, such as articulating arms, electro-mechanical elements, curved friction plates, etc. In some embodiments, as illustrated in FIG. 4B, the imaging system is configured to move in an isocenter manner. This can be used to enhance dexterity of the user of the system because hand-eye coordination is increased or maximized. Such enhanced dexterity can be vital for prolonged and/or difficult surgery. In the displayed embodiment, the horizons of the acquisition systems are configured to be horizontal to match the horizon of the display system and the user. As shown in FIG. 4B, in various embodiments, a stereo imaging system may be maintained in a horizontal configuration as it is moved across a range of locations to avoid confusion for the user viewing the video from the stereo camera. By maintaining a common relative horizon between the display and the acquisition system, the user can relatively easily translate hand motion to manipulation of objects in the display, which may not be the case where translation of the acquisition is accompanied by a relative rotation between the display and the acquisition system.

In the embodiments illustrated in FIGS. 4A and 4B, the isocenter assemblies can be a part of the display system or a separate, independent system. For example, the viewing platform 9 can be mounted on a separate arm from the cameras 18. Thus, the display and the image acquisition of the surgical imaging system can be decoupled, similar to the embodiment illustrated in FIG. 1. By decoupling the isocenter cameras 18 from the display ergonomic benefits are provided such as, for example, the surgeon does not need to be looking through binoculars for an extended period of time or at an uncomfortable position or angle. In various embodiments, a common relative horizon for both the display and the acquisition system may also be employed.

In some embodiments, the distance between the surgical site of interest and the imagers, e.g., the working distance, can be at least about 20 cm and/or less than or equal to about 450 cm, at least about 10 cm and/or less than or equal to about 50 cm, or at least about 5 cm and/or less than or equal to about 1 m, although values outside this range are possible.

The user can interact with the surgical imaging system 51 to select a working distance, which can be fixed throughout the procedure or which can be adjusted at any point in time. Changing the working distance can be accomplished using elements on a user interface, such as a graphical user interface, or using physical elements such as rotatable rings, knobs, pedals, levers, buttons, etc. In some embodiments, the working distance is selected by the system based at least in part on the cables and/or tubing being used in the surgical visualization system. For example, the cables and/or tubing can include an RFID chip or an EEPROM or other memory storage that is configured to communicate information to the surgical imaging system 51 about the kind of procedure to be performed. For an ENT/Head/Neck procedure, the typical working distance can be set to about 40 cm. In some embodiments, the user's past preferences are remembered and used, at least in part, to select a working distance.

In some embodiments, gross focus adjustment can be accomplished manually by positioning the cameras 18 and arm 7. The fine focus adjustment can be done using other physical elements, such as a fine focusing ring, or it can be accomplished electronically.

In some embodiments, the magnification of the surgical imaging system 51 can be selected by the user using physical or virtual user interface elements. The magnification can change and can range between about 1× and about 6×, between about 1× and about 4×, or between about 1× and about 2.5×. Embodiments may be able to change between any of these such as between 2.5× and 6× or between 2.5× and 6×. Values outside these ranges are also possible. For example, the system 51 can be configured to provide magnification and demagnification and image inversion, with a range from about −2× to about 10×, from about −2× to about 8×, from about −2× to about 4×, from about −0.5× to about 4×, or from about −0.5× to about 10×. The surgical imaging system 51 can be configured to decouple zoom features and focus adjustments, to overcome problems with traditional operating room microscopes. In some embodiments, the surgical visualization system 51 can be used to provide surgical microscope views. In some embodiments, the surgical imaging system 51 can decouple instrument myopia by providing an electronic display instead of a direct view of a scene. The electronic displays can be configured to be focused at varying levels of magnification allowing the user to view the displays without adjusting the oculars between magnification adjustments. Moreover, in various embodiments, the oculars can be configured to provide continuous views at infinity. In some embodiments, however, the principal user of the surgical imaging system may select an accommodation level for the oculars, rather than using a relaxed view provided by the electronic displays. The electronic displays, in various embodiments, however, can remain in focus and the ocular adjustments do not affect the focus of the various video acquisition systems. Thus, adjustments by the principal user do not affect the views of the other users of the system viewing, for example, other displays showing the video, as the cameras/acquisition systems can remain focused. In some embodiments, the surgical imaging system 51 can be focused at a relatively close working distance (e.g., a distance with a relatively narrow depth of field) such that the image remains focused when moving to larger working distances (e.g., distances with broader depth of field). Thus, the surgical imaging system 51 can be focused over an entire working range, reducing or eliminating the need to refocus the system after magnification or zoom adjustments are made.

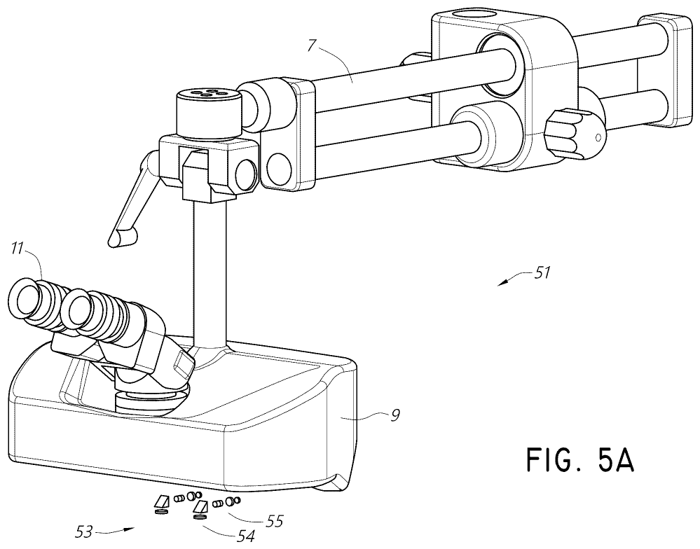

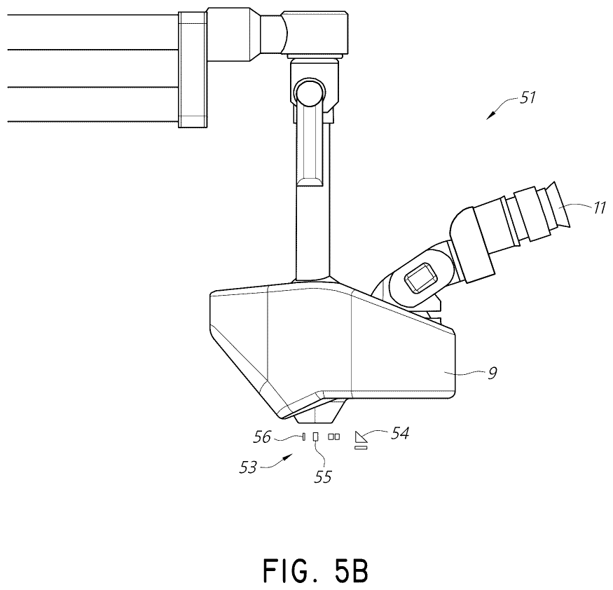

FIGS. 5A and 5B illustrate an embodiment of the surgical imaging system 51 having an optical system 53 mounted under the viewing platform 9. As illustrated, the optical components are shown as free-standing to show the structure of the components, but in practice the optical components 53 will be mounted within or on a structure attached to the viewing platform. In some embodiments, the optical system 53 and/or the cameras 18 (discussed above) can be modular and can be selected and swapped for use with the surgical imaging system 51. Paragraph from each of U.S. Prov. App. No. 61/880,808, U.S. Prov. App. No. 61/920,451, U.S. Prov. App. No. 61/921,051, U.S. Prov. App. No. 61/921,389, U.S. Prov. App. No. 61/922,068, and U.S. Prov. App. No. 61/923,188 is incorporated by reference herein.

The optical system 53 is configured to provide stereo image data to the imaging system 51. The optical system 53 includes a turning prism 54 to fold the optical path underneath the viewing platform 9 to decrease the physical extent (e.g., length) of the imaging system under the viewing platform 9.

In some embodiments, the optical system 53 comprises a Greenough-style system wherein the optical paths for each eye have separate optical components. In some embodiments, the optical system 53 comprises a Galilean-style system wherein the optical paths for each eye pass through a common objective. The Greenough-style system may be preferable where imaging sensors are being used to capture and convey the image data as compared to the Galilean-style system. The Galilean system can introduce aberrations into the imagery by virtue of the rays for each eye's optical path passing through a periphery of the objective lens. This does not happen in the Greenough-style system as each optical path has its own optics. In addition, the Galilean system can be more expensive as the objective used can be relatively expensive based at least in part on the desired optical quality of the lens and its size.

The optical system 53 can include two right-angle prisms 54, two zoom systems 55, and two image sensors 56. This folding is different from a traditional operating room microscope because the optical path leads to image sensors rather than to a direct-view optical system.

In some embodiments, the optical system 53 can have a relatively constant F-number. This can be accomplished, for example, by varying the focal length and/or aperture of the system based on working distance and/or magnification. In one embodiment, as the focal length changes, the eye paths can move laterally apart (or together), the prisms 54 can rotate to provide an appropriate convergence angle, and the apertures can change their diameters to maintain the ratio of the focal length to the diameter a relatively constant value. This can produce a relatively constant brightness at the image sensor 56, which can result in a relatively constant brightness being displayed to the user. This can be advantageous in systems, such as the surgical visualization systems described herein, where multiple cameras are being used and changing an illumination to compensate for changes in focal length, magnification, working distance, and/or aperture can adversely affect imagery acquired with other cameras in the system. In some embodiments, the illumination can change to compensate for changes in the focal length and/or the aperture so as to provide a relatively constant brightness at the image sensors 56.

The optical assembly 53 can include a zoom system 55 configured to provide a variable focal distance and/or zoom capabilities. A Galilean-style stereoscopic system generally includes a common objective for the two eye paths. When this optical system is imaged with image sensors 56, it can create aberrations, wedge effects, etc. that can be difficult to compensate for. In some embodiments, the surgical imaging system 51 can include a Galilean-style optical system configured to re-center at least one of the stereo paths to a central location through the objective lens, which can be advantageous in some applications.

In some embodiments, the real-time visualization system utilizes a Greenough-style system. This can have separate optical components for each stereo path. The optical assembly 53 can be configured to provide variable magnification and/or afocal zoom and can be configured to operate in a magnification range from about 1× to about 6×, or from about 1× to about 4×, or from about 1× to about 2.5×.

The distal-most portion of the Greenough assembly 53 can be similar in functionality to an objective lens of a typical, direct-view operating room microscope with the working distance set approximately to that of the focal length. The working distance, and in some implementations the focal length, can be between about 20 cm and about 40 cm, for example. In some embodiments the work distance may be adjustable from 15 cm to 40 cm or to 45 cm. Other values outside these ranges are also possible. In some embodiments, the surgical imaging system 51 includes an opto-mechanical focus element configured to vary the focal length of a part of the optical assembly 53 or the whole optical assembly 53.

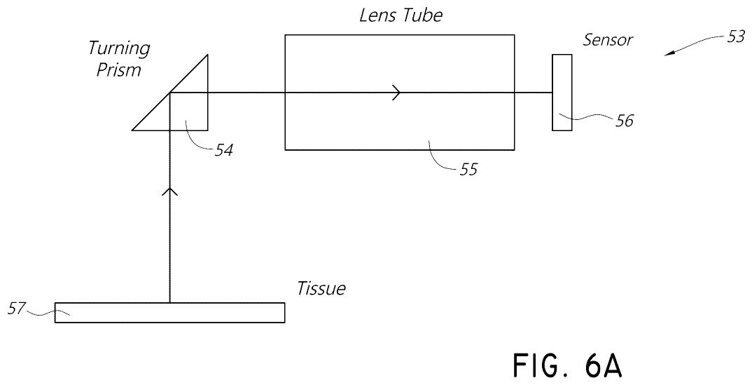

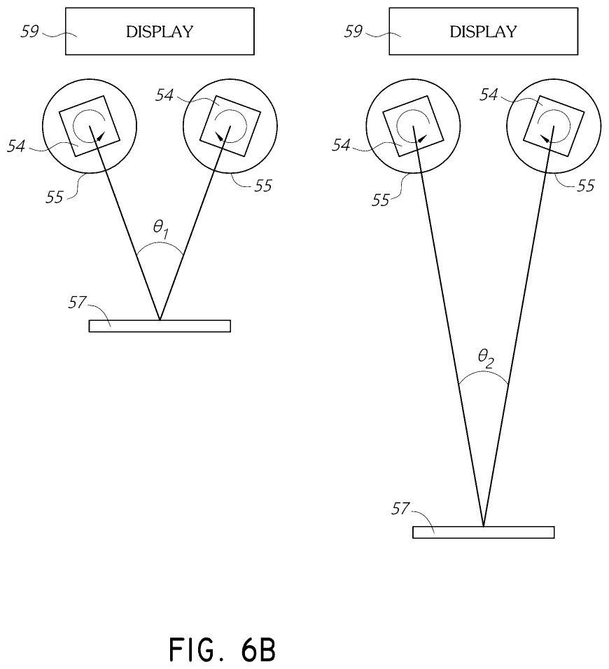

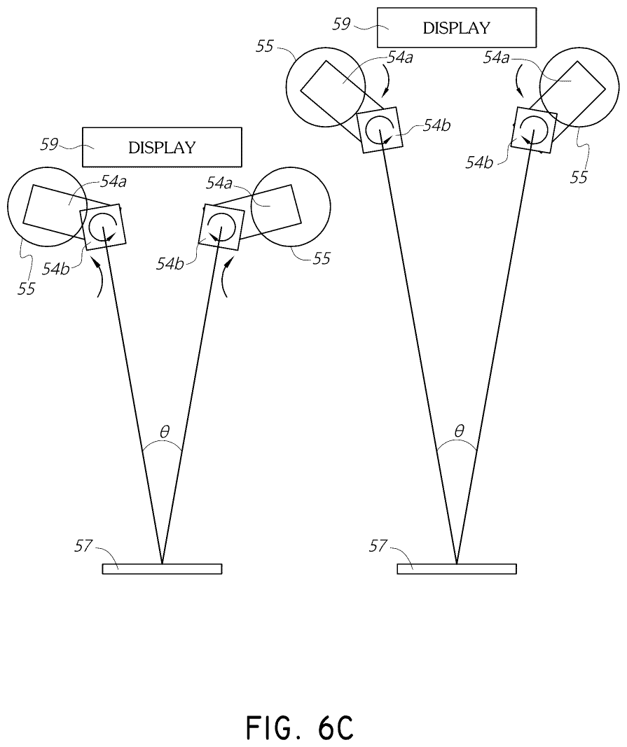

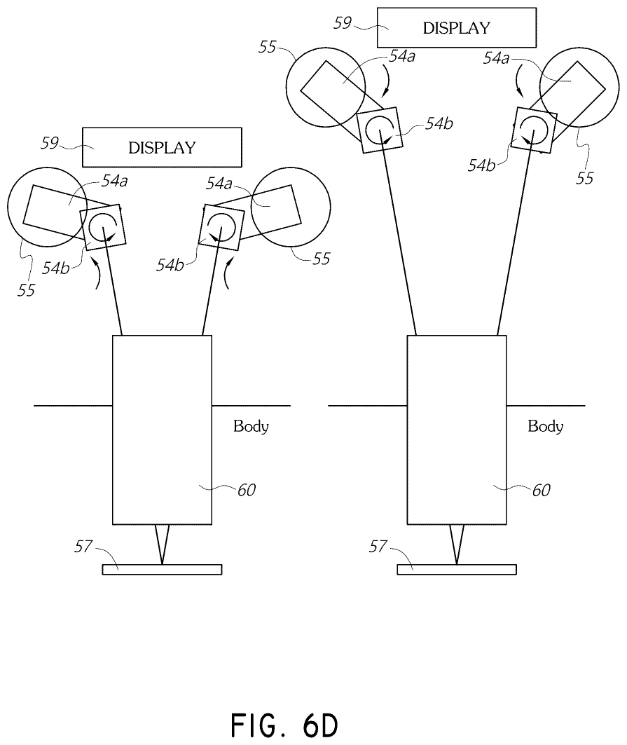

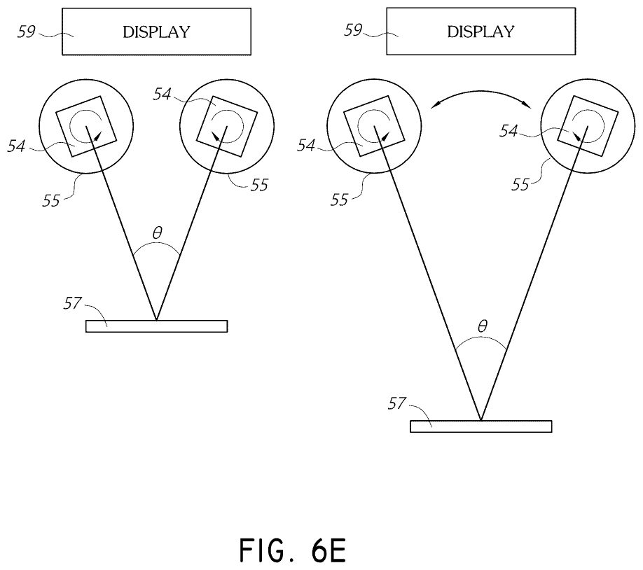

FIGS. 6A-6E illustrate embodiments of optical assemblies 53 for use in a stereoscopic surgical imaging system, such as those described herein with reference to FIGS. 5A-5B. FIG. 6A illustrates a side view of an example optical assembly 53 configured to use a turning prism 54 to fold an optical path from a tissue 57 to a sensor 56 along a lens train 55 that is situated near or adjacent to a viewing platform 9. This can advantageously provide a relatively long optical path in a relatively compact distance.

FIG. 6B illustrates a front view of an embodiment of an optical assembly configured to change a convergence angle in a stereoscopic imaging system. The prisms 54 can be the turning prism 54 illustrated in FIG. 6A. The prisms 54 can be configured to rotate to change a convergence angle, and as a result, a convergence point and/or a working distance. The working distance, which can be a distance from the prisms 54 to the target 57 (e.g., tissue), can be user-selectable or adjustable. In various embodiments, with increased working distance to the target 57, the convergence angle can decrease. Conversely, when the working distance gets smaller, the convergence angle can increase (e.g., θ1>θ2). This can be advantageous where the lens path 55 is fixed and the working distance is adjustable. The stereo imagery can then be viewed on the display 59 by a user.

FIG. 6C illustrates a front view of an embodiment of an optical assembly 53 that is configured to maintain a substantially constant convergence angle. The optical assembly 53 can include two prisms 54a and 54b for each optical path, wherein the prisms 54a, 54b can move and/or rotate. For example, when the working distance decreases the first set of prisms 54a can rotate towards one another to decrease an effective distance between the second set of prisms 54b. The second set of prisms 54b can, in turn, rotate to compensate for the changed angle so as to converge on the common target. The second set of prisms 54b can direct the light to the first set of prisms 54a which can then direct the light down the fixed lens paths 55 (e.g., fixed in their position relative to the viewfinder). By providing a relatively fixed convergence angle, a change in working distance may not require refocusing for the user. Maintaining a constant convergence angle, especially a comfortable angle, may reduce the strain on the user such as a surgeon performing a prolonged, arduous procedure.

FIG. 6D illustrates a front view of an embodiment of an optical assembly 53 configured to provide a substantially narrow convergence angle to be able to view stereoscopic imagery through a narrow insertion tube 60 (e.g., a tube partially inserted into a body during a procedure). A similar assembly 53 can be used as described with reference to FIG. 6C, and the convergence angle can be maintained substantially constant or at least sufficiently narrow to view through the insertion tube 60.

FIG. 6E illustrates a front view of an embodiment of an optical assembly 53 configured to provide a substantially constant convergence angle by moving the lens paths 55 laterally, e.g., toward or away from one another. The prisms 54 can be made to have a substantially constant orientation (e.g., no rotation for changing working distances) and compensation for changing working distance can be accomplished by translating the optical paths laterally to separate or join the optical paths. The translation of the optical paths can be accomplished using any suitable means including, for example, electro-mechanical actuators, slides, articulating arms, etc. This can simplify the optical assembly compared to the embodiments having two sets of prisms as only one set of prisms may be used when the lens paths are configured to move.

The embodiments of the optical assembly 53 which are configured to maintain a sufficiently narrow convergence angle can be advantageous as they allow stereo access to narrow surgical entries by allowing the angle to decrease and avoid clipping one of the stereo paths. For example, the left and right lens paths can move closer to one another and the prisms can adjust to the proper convergence angle for that distance. As another example, the left and right lens paths can remain fixed and there can be sets of prisms for each path configured to direct the light along the lens paths while maintaining a substantially constant convergence angle. In some embodiments, maintaining a constant convergence angle can be visually helpful to the user when zoom changes, e.g., because the changing depth cues do not confuse the user's eye and/or brain. In addition, constant convergence may induce less stress on the user.

### Movement Control System

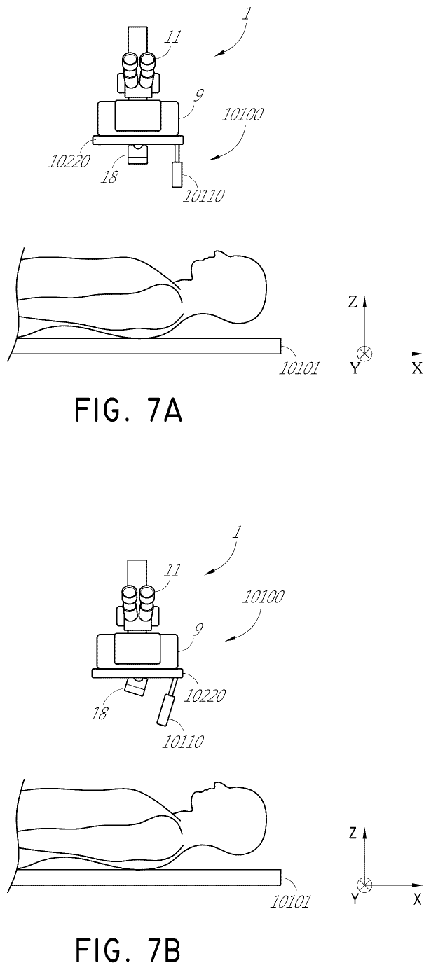

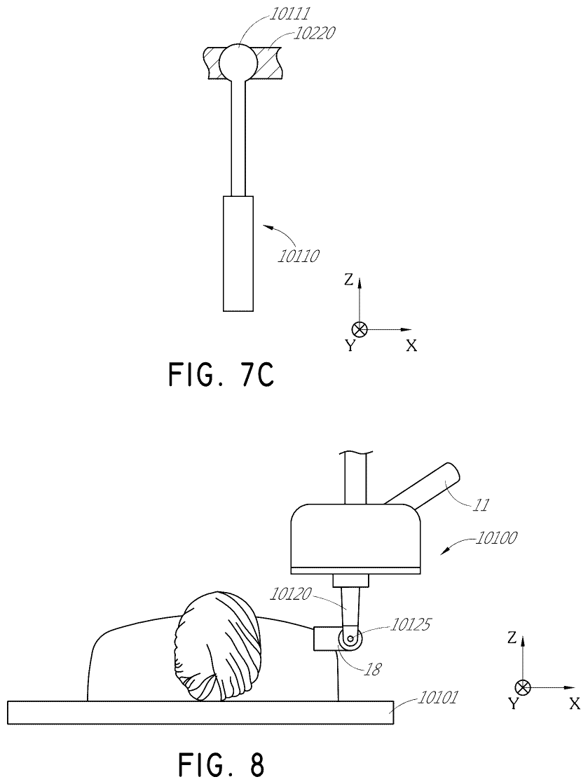

FIGS. 7A-C illustrate embodiments of components of a movement control system 10100 that can be configured to allow an operator of the surgical visualization system 1, such as a medical professional or assistant, to control the movement of one or more imagers 18. Such imagers may comprise cameras that provide a surgical microscope view through the oculars 11 or eyepieces of the binocular display unit 9. In various embodiments, the movement control system can enable the imagers 18 to be moved without changing the positioning of oculars 11, and thus an operator can remain in an ergonomic position while changing the view provided by the imager 18. The imager 18 can be on the binocular display unit 9 or located elsewhere such as on a separate platform or articulated arm. Additionally, unlike conventional articulated optical systems which are generally unwieldy, complex, and have the potential for introducing optical aberrations, use of the movement control system 10100 with the surgical visualization system 1 can result in a simplified system with greater optical clarity and range of movement. It should be appreciated by one of skill in the art that, while the description of the movement control system 10100 is described herein in the context of medical procedures, the movement control system 10100 can be used for other types of visualization and imaging systems. Movement of the imagers 18 can be performed prior to and/or during the activity, such as surgical procedures, dental procedures, and the like. Movement of the imagers 18 can advantageously allow a medical professional or other operator to alter the view through oculars 11, for example, to provide different surgical microscope-like electronic visualizations which might be beneficial during the course of a medical procedure or for different surgical procedures.

In some embodiments, control of the movement of the imager 18 can be achieved using a single control member such as 10110. This provides the advantage of allowing single-handed operation of the movement control system 10100 which can, for example, allow a medical professional to move one or more imagers 18 using only one hand while using a second hand for other tasks such as performing surgical techniques. It should be appreciated by one of skill in the art that, while the description of the movement control system 10100 is described herein in the context of medical procedures, the movement control system 10100 can be used for other types of visualization and imaging systems.

### Operation

As illustrated in FIGS. 7A-C, in some embodiments, the control member, such as a joystick, 10110 can be used to translate the imager 18, adjust the pitch, yaw, and/or roll of the imager 18, and/or adjust the working distance of the imager 18. In some embodiments, the oculars 11 can remain immobile when translating the imager 18, adjusting the pitch, yaw, and/or roll of the imager 18, and/or adjusting the working distance of the imager 18. The ability for a single control member 10110 to control translation, adjustments to pitch and/or yaw, and/or adjustments to the working distance can beneficially simplify operation of the device as an operator need not release the control member 10110 to control multiple aspects of its operation. For example, an operator can translate the imager 18 and subsequently adjust the pitch and/or yaw without having to release the control member 10110 thereby increasing ease-of-use of the system and enhancing efficiency when using this system. In some embodiments, however, a pair of control members 10110 (e.g., a left hand control member and a right hand control member) can be used for left hand and right hand.

As shown in FIG. 7C, one or more control members of the movement control system 10100, such as control member 10110, and/or one or more imager arms (see FIG. 8) can be attached to a component of the movement control system 10100 using various types of joints and/or can be remote from the movement control system 10100 such as a remote joystick or toggle. In some embodiments, the control member 10110 can include a joint for attachment to the movement control system 10100. For example, as shown in the illustrated embodiment, control member 10110 can include joint 10111. In some embodiments, one or more of the joints can include components for detecting movement of the control member and/or an imager arm. For example, one or more of the joints can include one or more sensors for detecting rotation and/or translation of the control member and/or the imager arm about the joint. The signals from these sensors can be used to control other components of the movement control system, such as one or more electromechanical components.

For purposes of this disclosure, rotation about joints, such as joint 10111, around the x-axis is hereinafter termed “pitch” or “tilt” and rotation about joints, such as joint 10111, around the y-axis is hereinafter termed “yaw” or “pan.”

As shown in the illustrated embodiment, the joint 10111 can be spherical joints received in a socket formed in the member 10220 thereby forming a ball-and-socket attachment. As should be apparent to one of ordinary skill in the art, other types of mounting mechanisms may be used for attaching control member 10110 as well as an imager arm to components of the movement control system 10100. For example, joints such as gimbals can be used which limit the rotational degrees of freedom about the gimbal. Other types of joint can be used depending on the types of movement the movement control system is designed to allow. For example, if only pitch is needed without yaw, one can use a joint having a single rotational degree of freedom. In some embodiments, the control member 10110 can be positioned remotely from the movement control system 10100.

### General Embodiment

With continued reference to FIGS. 7A and 7B, in some embodiments, the movement control system 10100 can be attached to an attachment structure, such as binocular display unit 9, and support one or more imagers 18. As shown in the illustrated embodiment, the movement control system 10100 can be oriented generally underneath the binocular display unit 9 and in some embodiments can be sized such that the movement control system 10100 does not extend significantly beyond the outer housing of the binocular display unit 9. This can advantageously provide a smaller form factor thereby reducing the likelihood that the movement control system 10100 will interfere with the medical professionals and assistants during a medical procedure. In other embodiments, the attachment structure can be other components of the surgical visualization system 1 such as, but not limited to, a dedicated articulating arm or a display arm. In some embodiments, the movement control system 10100 can extend significantly beyond the outer housing of the binocular display unit 9 or any other platform to which it is attached. This can be advantageous in situations where a greater degree of movement of the imagers 18 is desired or in embodiments where the control member 10110 is located above the attachment point between the movement control system 10100 and binocular display unit 9.

With continued reference to FIGS. 7A and 7B, as discussed in part above, the movement control system 10100 can be configured to allow translation of one or more attached imagers 18 along a plane relative to the binocular display unit 9. In some embodiments, the binocular display unit 9 can be immobile while the one or more imagers 18 are translated. For example, when attached to the binocular display unit 9 with the movement control mechanism 10100 parallel to an operating table 10101, the one or more imagers 18 can be translated along a plane parallel to the operating table 10101. As shown in the illustrated embodiment, the movement control system 10100 can be translated along both the x-axis and the y-axis (which projects perpendicularly through the sheet). This can advantageously allow the medical professional to position the view of oculars 11 for comfortable viewing by the surgeon thereby reducing physical strain on the surgeon during long procedures.

In some embodiments, defining an imager 18 centered on the movement control system 10100 (as shown in FIG. 7A) as having an x-axis, y-axis, and z-axis coordinate of zero, the movement control system 10100 can have a range of translation relative to the binocular display unit 9, of approximately ±500 mm along the x-axis and y-axis at full extension, approximately ±400 mm along the x-axis and y-axis at full extension, approximately ±300 mm along the x-axis and y-axis at full extension, approximately ±200 mm along the x-axis and y-axis at full extension, or approximately ±100 mm along the x-axis and y-axis at full extension. In some embodiments, full extension along one axis can be greater than full extension along the other axis. For example, in some embodiments, full extension along the x-axis may be approximately ±175 mm whereas the y-axis extension can be three-quarters full extension of the x-axis, one-half full extension of the x-axis, one-quarter full extension of the x-axis, or any other ratio between unity and zero. In some embodiments, the range of translation relative to the binocular display unit 9 along the y-axis can be approximately ±87.5 mm. This can be advantageous in cases where allowing the y-axis to have a full range of motion may interfere with the medical professional and/or assistants.

These ratios can be reversed such that the range of translation of the x-axis can be three-quarters full extension of the y-axis, one-half full extension of the y-axis, one-quarter full extension of the y-axis, or any ratio between unity and zero. Additionally, in some embodiments, the imager 18 can translate further in the “positive” direction than the “negative” direction. For example, along the x-axis, the imager 18 may move from −100 mm to 500 mm. Ranges of motion outside these ranges are also possible. As should be apparent to one of ordinary skill in the art, the maximum translation relative to the binocular display unit 9 along the x-axis and y-axis can be chosen to provide a balance between greater maneuverability, the yaw and/or pitch angles, working distances, size constraints, and other such factors.

As described in part above and as will be discussed in greater detail below, in some embodiments, translation of the imagers 18 can be performed by translating one or more control members, such as control member 10110, in the desired direction. In some embodiments, the control member 10110 can be electrically coupled to the movement control system 10100 to provide translation via an electromechanical system utilizing stepper motors, linear motors, or the like. For example, a joint of the control member 10110 can include components for detecting translation of the control member 10110. The signals from these sensors can be used to control other components of the movement control system, such as one or more electromechanical components such as stepper motors, linear motors, or the like to translate the imager 18. The electromechanical components can be coupled to a moveable platform to which the imager 18 can be attached. In some embodiments, the control member 10110 can be physically connected to the movement control system 10100 without any electromechanical assistance.

As should be appreciated by one of ordinary skill in the art, the movement control system 10100 need not translate solely along a plane parallel to the operating table 10101 or the x-y plane as set forth in the illustrated embodiment. In some embodiments, the plane of translation can be defined by the orientation of the mount to which the movement control system 10100 is connected. In some embodiments, the movement control system 10100 can be configured for non-planar translation and/or translation along more than one plane. In some embodiments, for example, a tip and tilt stage provides angular motion. A rotary stage can also be used to provide rotary motion.

With continued reference to FIGS. 7A and 7B, as described in part above, the movement control system 10100 can be configured to allow rotation of the one or more attached imagers 18 about a joint which can be attached to components of the movement control system 10100 and/or remotely from the movement control system 10100. In some embodiments, the movement control system 10100 can be designed to allow the control member, such as control member 10110, as well as the imager 18 and/or imager armto “pitch” or “tilt” and “yaw” or “pan” relative to the binocular display unit 9. In some embodiments, the binocular display unit 9 can be immobile while the “tilt” and “yaw” or “pan” of the one or more imagers 18 are adjusted. Pitch or yaw can allow the imager 18 to have a line of sight that is centered (e.g., focused) on the surgical site after the imager 18 is translated. This can advantageously allow the medical professional or assistant to adjust the viewing angle during a medical procedure. This can be beneficial in circumstances where a medical professional is unable to adequately view an object due to another element obstructing the view. Under such circumstances, a medical professional can translate the imager 18 and adjust the viewing angle of the imager 18 such that the same general area is viewed from a different angle.

In some embodiments, defining an imager 18 in a perpendicular orientation to the movement control system 10100 (as shown in FIG. 7A) as having an a pitch and yaw of zero (i.e., as shown in FIG. 7A), the movement control system 10100 can allow both pitch and yaw adjustments relative to the binocular display unit 9 within the range of approximately #60 degrees each, by approximately ±50 degrees each, by approximately ±40 degrees each, by approximately ±30 degrees each, by approximately ±20 degrees each, or approximately ±10 degrees each. In some embodiments, the pitch and yaw can have different adjustment ranges. For example, in some embodiments, the yaw can have an adjustment range of approximately ±40 degrees whereas the pitch can have an adjustment range of approximately three-quarters that of the yaw, one-half that of the yaw, one-quarter that of the yaw, or any other ratio between unity and zero. In some embodiments, the pitch can have an adjustment range of approximately ±20 degrees.

The adjustment range of yaw and pitch can correspond to the distance at full extension along both the x-axis and the y-axis. For example, in some embodiments, the pitch and yaw can be chosen such that the imager 18 can remain centered on the surgical site when the movement control system 10100 is fully extended in any direction. In some embodiments, the working distance between the imager 18 and the surgical site can be approximately 200 mm, with a range of translation along the x-axis of approximately ±175 mm, and a range of translation along the y-axis of approximately ±87.5 mm. In order to remain centered on the surgical site, the pitch adjustment range can be ±20 degrees and the yaw adjustment range can be ±40 degrees. As such, because the full extension need not be the same in both directions, the pitch and yaw adjustment ranges can also be different to match the differences in extension. In other embodiments, such as those in which the working distance can be adjusted, the pitch and yaw adjustment range can be chosen such that the imager 18 can remain centered on the surgical site when the movement control system 10100 is fully extended in any direction at at least one working distance. For example, in embodiments where the working distance can be adjusted between approximately 200 mm and 400 mm, the pitch and yaw adjustment range can be approximately ±20 degrees and approximately ±10 degrees respectively to allow centering at a working distance of 400 mm.

Additionally, in some embodiments, the imager 18 can adjust further in a “positive” angle than a “negative” angle. For example, the yaw may range from −5 degrees to 15 degrees.

As described in part above and as will be discussed in greater detail below, in some embodiments, increasing or decreasing the pitch and/or yaw of the imagers 18 relative to the binocular display unit 9 can be achieved by increasing or decreasing the pitch and/or yaw of the one or more control members, such as control member 10110. In some embodiments, the control member 10110 can be electrically coupled to the movement control system 10100 to provide pitch and yaw via an electromechanical system utilizing stepper motors, linear motors, or the like. For example, a joint of the control member 10110 can include components for detecting pitch and/or yaw of the control member 10110. In some embodiments, the joint of the control member 10110 can be gimbals which can detect pitch and/or yaw of the control member 10110. The signals from these sensors can be used to control other components of the movement control system, such as one or more electromechanical components such as stepper motors, linear motors, or the like to adjust the pitch and/or yaw of the imager 18. As should be appreciated by one of ordinary skill in the art, in some embodiments, the movement control system 10100 can be configured to allow rotation along other axes such as the z-axis. In some embodiments, the control member 10110 can be physically connected to the movement control system 10100 without any electromechanical assistance.

Additionally, in some embodiments, the movement control system 10100 can be configured to adjust the working distance between the imagers 18 and the surgical site. In some embodiments, the binocular display unit 9 can remain immobile while the working distance of the imagers 18 are adjusted. In some embodiments, the working distance can range from between approximately 1 m to approximately 10 mm, from between approximately 800 mm to approximately 50 mm, from between approximately 600 mm to approximately 100 mm, or from between approximately 400 mm to approximately 200 mm. In some embodiments, the control member 10110 can be electrically coupled to the movement control system 10100 to provide working distance adjustment via an electromechanical system utilizing stepper motors, linear motors, or the like. For example, a joint of the control member 10110 can include components for detecting rotation of the control member 10110 about the longitudinal axis. The signals from these sensors can be used to control other components of the movement control system, such as one or more electromechanical components such as stepper motors, linear motors, or the like to adjust the pitch and/or yaw of the imager 18. In some embodiments, the control member 10110 can be physically connected to the movement control system 10100 without any electromechanical assistance.

In some embodiments, the movement control system 10100 can include a translation system for translating an imager 18 and/or an imager arm, a pitch-yaw adjustment system for adjusting the pitch and/or yaw of the imager 18 and/or an imager arm, a control member, such as control member 10110, and one or more imager arms to which the imager 18 can be attached. In some embodiments, a working distance adjustment system can be included which can allow adjustments in working distance of the imager 18 and/or an imager arm. It should be appreciated by one of ordinary skill in the art that the translation system, the pitch-yaw adjustment system, and/or the working distance adjustment system can be used separately or in any combination.

Operation of the translation, pitch-yaw adjustment, and/or working distance adjustment systems can be performed using a control member, such as control member 10110. In some embodiments, control member 10110 can be operatively coupled to the translation, pitch-yaw adjustment, and/or working distance adjustment systems. For example, as described above, in some embodiments, the control member can be coupled to an electromechanical system for controlling the translation, pitch-yaw adjustment, and/or working distance adjustment systems. The control member can be directly attached to a component of the movement control system 10100 or can be remotely positioned (e.g., a toggle or joystick on a separate module). In some embodiments, the control member can be coupled directly to the translation, pitch-yaw adjustment, and/or working distance adjustment systems such that no electromechanical devices are used. In some embodiments, the operator can be given the option of controlling the translation, pitch-yaw adjustment, and/or working distance adjustment systems with or without electromechanical devices. For example, the operator can control the translation, pitch-yaw adjustment, and/or working distance adjustment systems without electromechanical devices for certain portions of a procedure and use such electromechanical devices for controlling the translation, pitch-yaw adjustment, and/or working distance adjustment systems during other portions of a procedure. As another example, in some embodiments coarse control of the movement control system 10100 can be achieved without use of electromechanical devices whereas fine control of the movement control system 10100 can be achieve with use of electromechanical devices, vice-versa, or a combination of the two.

In some embodiments, the movement control system 10100 can include a control system which controls functions of the electromechanical devices. In some embodiments, the electromechanical components can be programmed such that the electromechanical components can orient the translation, pitch-yaw adjustment, and/or working distance adjustment systems in certain positions based on the operator's input. For example, the electromechanical components can be programmed such that it goes to reverts back to a pre-set or previous position upon receiving a command from the operator. As another example, the electromechanical components can be programmed such that an operator can specify a desired position for the imager 18 and the control system can control the electromechanical devices coupled to the translation, pitch-yaw adjustment, and/or working distance adjustment systems orient the imager 18 in the desired position.

With reference to FIG. 8, in some embodiments, the imager arm 10120 and the imager 18 can be attached such that the imager 18 can be directed towards the side of the head of a patient. For example, in some embodiments, the imager 18 can be attached to the imager arm 10120 using a yoke 10125 which can be designed to allow for coarse and/or fine control of pitch, yaw, and/or roll of the imager 18. In some embodiments, the yoke 10125 can have one or more pivots which can be configured to allow the imager 18 to have a viewing angle parallel to the operating room floor such that an operator can view the side of the head. In some embodiments, the yoke 10125 can be configured to allow the imager 18 to rotate such that the imager can be directed to a portion of the back of the head.

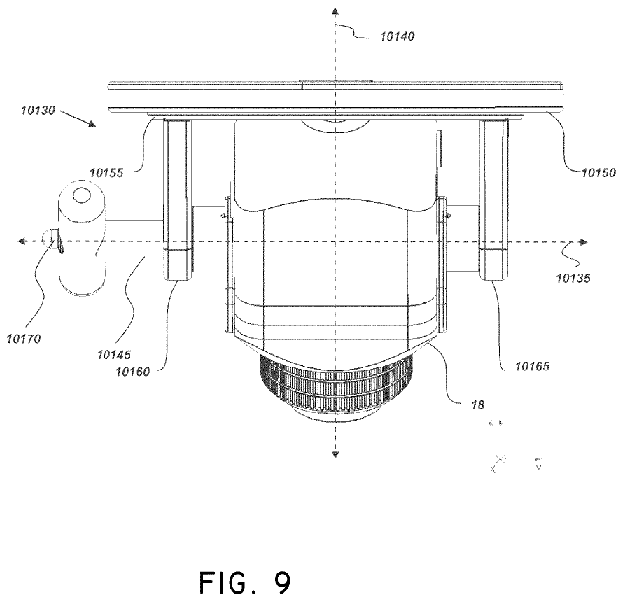

In some embodiments, the imager 18 can be positioned on a movement control system 10130 providing at least two rotational degrees of freedom and/or at least one translational degree of freedom. In some embodiments, movement control system 10130 can provide two rotational degrees of freedom and at least two translation degrees of freedom. For example, as shown in FIG. 9, the movement control system 10130 can allow for rotation along axis 10135 of the movement control system 10130 and/or along axis 10140 (which can be parallel with the z-axis). Moreover, as shown in the illustrated embodiment, the movement control system can allow translation along both the x-axis and y-axis. In some embodiments, apparatus 10130 can provide at least one translational degree of freedom.

As shown in the illustrated embodiment, the movement control system 10130 can include a one or more control members, such as control member 10145. Control member 10145 can be positioned such that the longitudinal axis of the control member 10145 is parallel with and/or collinear with axis 10135. This can advantageously allow the imager 18 to be rotated about axis 10135 by rotating the control member 10145. In some embodiments, the control member 10145 can be mechanically coupled to the imager 18. In some embodiments, the control member 10145 can be coupled to the imager 18 via an electromechanical system. For example, the control member 10145 can include sensors for detecting rotation of the control member 10145 and use data received from the sensors to rotate the imager 18 via electromechanical components such as stepper motors, linear motors, or the like.

As shown in the illustrated embodiment, the movement control system 10130 can include a first plate element 10150 and a second plate element 10155 which can be rotatable coupled. The second plate element 10155 can include first and second supports 10160, 10165 to which the imager 18 can be attached. In some embodiments, the first and second plate elements 10150, 10155 can be rotatable coupled such that the axis of rotation of the two plate elements 10150, 10155 is parallel and/or collinear with axis 10140.

In some embodiments, the control member 10145 can include one or more switches and/or actuators 10170 for controlling movement of the device. For example, the actuator 10170 can be coupled to mechanisms which can unlock the apparatus 10130 such that the movement control system 10130 can be manipulated to rotate and/or translate the imager 18. In some embodiments, the switches and/or actuators can be coupled to an electromechanical system to rotate and/or translate the movement control system 10130.

In some embodiments the movement control system 10130 may comprise a plurality of handles, for example, a left hand handle and a right hand handle to accommodate for surgeons who are left handed and surgeons who are right handed.

Also, the movement control system can be placed on a different platform other than the binocular display unit to further decouple the line of sight of the microscope view camera and the surgeon. For example, various embodiments may include at least two arms, one for a binocular display unit and one for a camera or imager that provides stereo surgical microscope views and to allow the surgical microscope camera be placed at a location away from the binocular display unit and to decouple line of sight of the surgical microscope from the line of sight of the ocular.

### Optical Systems for Displays

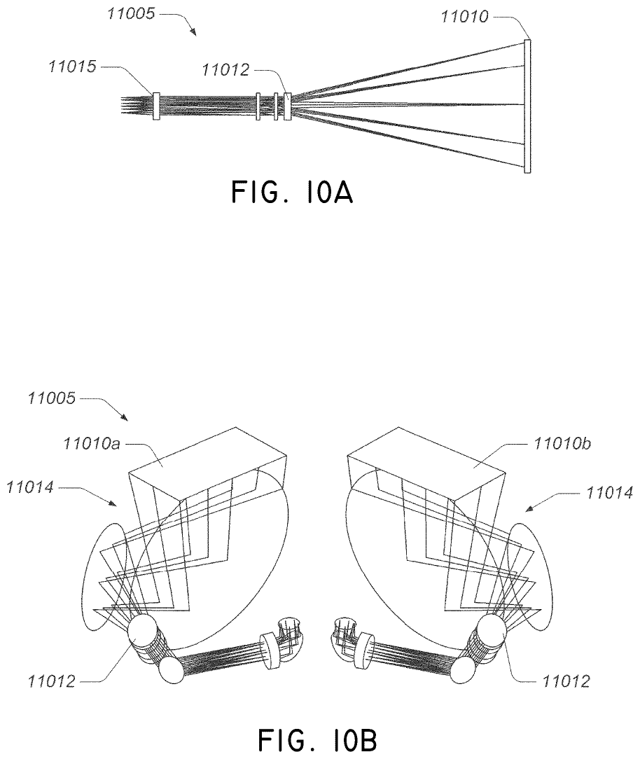

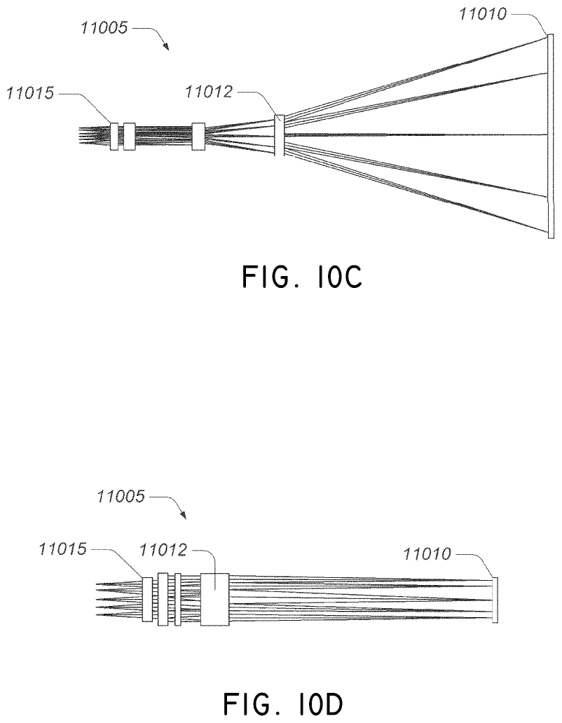

FIGS. 10A-10D illustrate example display optical systems 11005 configured to provide a view of displays 11010 through oculars (not shown) that receive light from the last lens 11015 in the display optical system 11005. The display optical system 11005 forms an exit pupil at or near the entrance pupil of the surgeon binoculars. These pupils are closely matched, for example, in size and shape. In some embodiments, the exit pupil of the display optical system 11005 can be the same size or smaller than the entrance pupil of oculars used to view the display. The oculars form an exit pupil that is matched (e.g., in size and shape) to the entrance pupil of the surgeon's eye(s). In some embodiments, the display optical system 11005 is configured to produce a beam that has a relatively constant cross-section between the first lens element 11012 and the last lens element 11015, where the cross-section is relatively small. Advantageously, this allows the display optical system 11005 to be included in a relatively small or compact package and use relatively small optical elements. In some embodiments, the last lens 11015 collimates the beam leaving the display optical system 11005. The termination of the rays shown in FIG. 10A to the left of lens 11015 is the exit pupil of the display optical system 11005. In some embodiments, the exit pupil of the display optical system 11005 is configured to be the same size or smaller than, and positioned at the same location, as an entrance pupil of a binocular viewing assembly configured to allow a user to view the display 11010.

The lenses in the display optical system 11005 form a highly color-corrected view of the display by forming the exit pupil in a position favorably disposed for the user and the binoculars. A combination of singlets and bonded lenses provide such correction. The display optical system 11005 may be designed to provide such correction while keeping a small beam column or ray bundle, which permits adding mirrors and obtaining a compact package. In various embodiments, producing an undistorted image can be difficult without such a group of lenses designed properly to provide such correction. This correction includes both color correction as well as distortion correction.

The display optical system 11005 advantageously allows a relatively small, compact lens assembly to provide a view of a relatively large display 11010. The display optical system 11005 can be configured to work with displays 11010 of varying sizes, including, without limitation, displays with a diagonal that is less than or equal to about 0.86 in. (22 mm), at least about 0.86 in. (22 mm) and/or less than or equal to about 10 in., at least about 1 in. and/or less than or equal to about 9 in., at least about 2 in. and/or less than or equal to about 8 in., or at least about 4 in. and/or less than or equal to about 6 in. The display may, for example, have a diagonal of about 5 inches or about 8 inches in some embodiments. The total optical path length of the display optical system 11005 can be less than or equal to about 9 in., at least about 9 in. and/or less than or equal to about 20 in., at least about 10 in. and/or less than or equal to about 19 in., at least about 14 in. and/or less than or equal to about 18 in. The display optical system 11005 can include lenses, mirrors, prisms, and other optical elements configured to direct and manipulate light along an optical path. The display optical system 11005 can be used in conjunction with a primary display, a surgeon display, an assistant display, possibly other displays, or any combination of these.

The example display optical system 11005 illustrated in FIG. 10A has a total optical path length of about 16.2 in. (412 mm). It is configured to provide an image of a 5 in. display 11010. The display optical system 11005 can include a lens 11012 configured to direct the light from the display 11010 along a path wherein light from the display 11010 is directed along a path with a relatively narrow cross-section. In various embodiments, the light received from the display is initially substantially reduced in beam size for example by the lens 11012 or lenses closest to the display and a more narrow beam is produced. In certain embodiments, for example, the lens 11012 or lenses closest to the display collect light at an angle (half angle) in excess of 20°, 25°, 30° and reduce the beam size of the light. This design is advantageous because it allows for the elements in the display optical system 11005 to be relatively small and compact. In some embodiments, the cross-section of the optical beam after the lens 11012 in the display optical system 11005 can be configured to be relatively constant. This configuration allows folding or redirecting mirrors present in the optical path to remain small.

FIG. 10B illustrates a binocular display optical system 11005 configured to provide a view of stereo displays 11010a, 11010b through a pair of oculars. The binocular display optical system 11005 can be based on the optical design illustrated in FIG. 10A, and can include one or more elements 11014 in the optical path before the lens 11012 to reduce the physical size of the optical system while maintaining the length of the optical path. These elements can include mirrors, prisms, and/or other optical elements configured to redirect the light from the displays 11010a, 11010b to the lens 11012. In some embodiments, the elements 11014 include curved mirrors which redirect the optical path and converge the rays from the displays 11010a, 11010b. In some embodiments, the elements 11014 include mirrors or prisms (for example that may have planar reflecting surface) that do not substantially affect the convergence of the light rays, but redirect the optical path. In some embodiments, because of the shape of the beam incident on the reflective surface, for example, mirror, the reflective surface or cross-section of the mirror is non-circular, and is, for example, elliptical. Accordingly, in various embodiments the cross-section of the mirror or other reflective surface is possibly being longer in one direction than in another, for example, orthogonal direction. These elements may fold the optical path to provide for a more compact system. Such a system may therefore have an optical path length from display to ocular that is longer than the length and/or width of the viewing platform of the combination thereof.

In some embodiments, the display optical system 11005 can include at least four mirrors, or less than or equal to four mirrors. In certain implementations, two mirrors can be used to fold the optical path from the display 11010 to the exit pupil, the two mirrors positioned between the first lens 11012 and the display 11010. In some embodiments, the display optical system 11005 includes at least four lenses or less than or equal to four lenses.

The example display optical system 11005 illustrated in FIG. 10C has a total optical path length of about 18.7 in. (475 mm). It is configured to provide an image of an 8 in. display 11010. The display optical system 11005 can include a lens 11012 configured to direct the light from the display 11010 along a path wherein light from the display 11010 is directed along a path with a relatively narrow cross-section, allowing for the display optical system 11005 to be relatively small and compact. In some embodiments, the cross-section of the optical beam after the lens 11012 in the display optical system 11005 (e.g., to the exit pupil prior to the entrance to a binocular viewing assembly) can be configured to be relatively constant. This configuration allows folding or redirecting mirrors present in the optical path to remain small. The display optical system 11005 can be configured to be used in conjunction with a display 11010 with a relatively high resolution.

The example display optical system 11005 illustrated in FIG. 10D has a total optical path length of about 9.3 in. (237 mm). It is configured to provide an image of a smaller display, in this case a 0.9 in. (22 mm) display 11010. Because the display is much smaller than the display in the embodiments described in connection with FIGS. 10A-10C, the optical path can be much shorter and may fit into a smaller space. The display optical system 11005 can include a lens 11012 configured to direct the light from the display 11010 along a path wherein light from the display 11010 is directed along a path with a relatively narrow cross-section, allowing for the display optical system 11005 to be relatively small and compact. In some embodiments, the cross-section of the optical path after the lens 11012 in the display optical system 11005 can be configured to be relatively constant. This configuration allows folding or redirecting mirrors present in the optical path to remain small. Based at least in part on the relatively short optical path length, the display optical system 11005 can be configured to be used in conjunction with a secondary display or an assistant display.

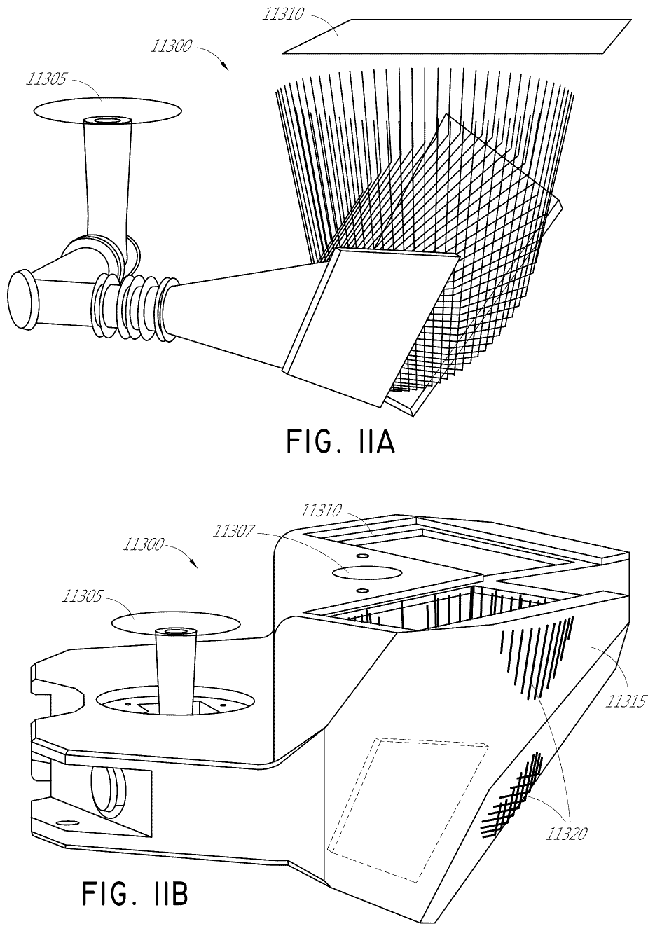

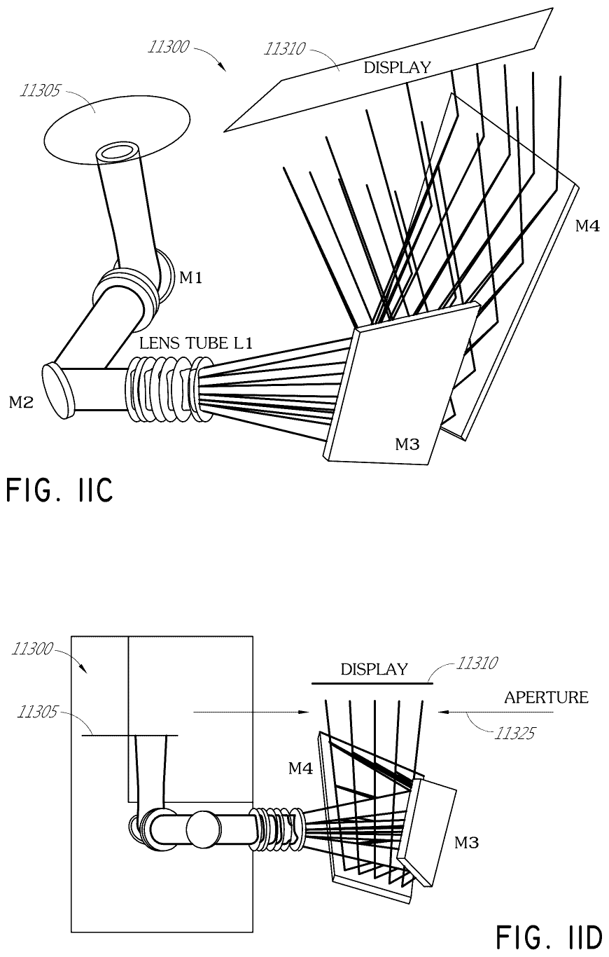

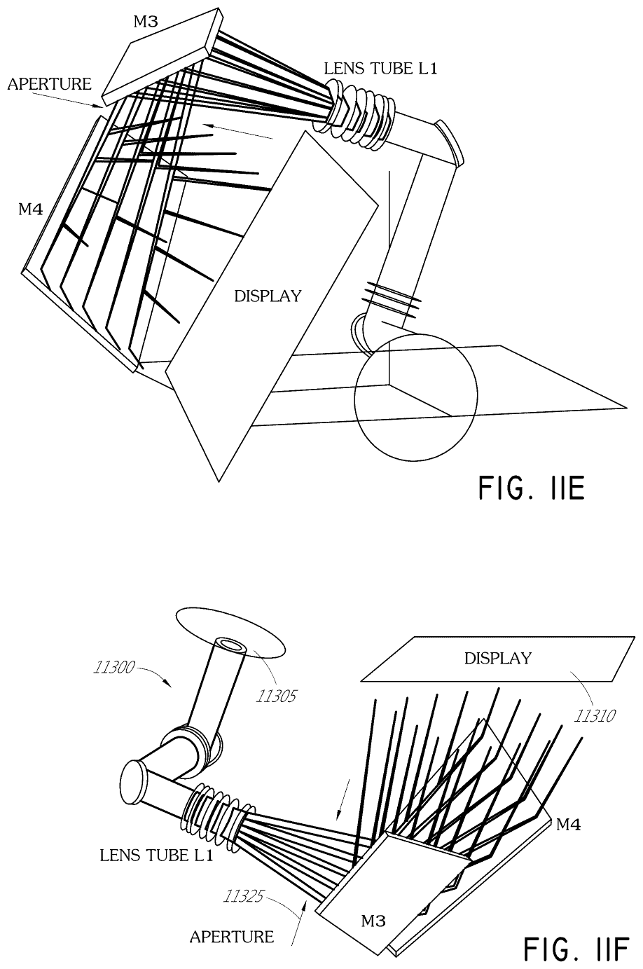

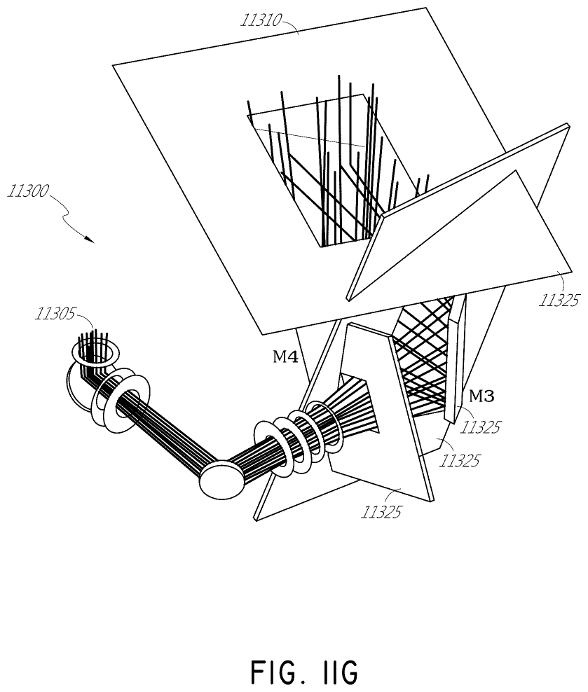

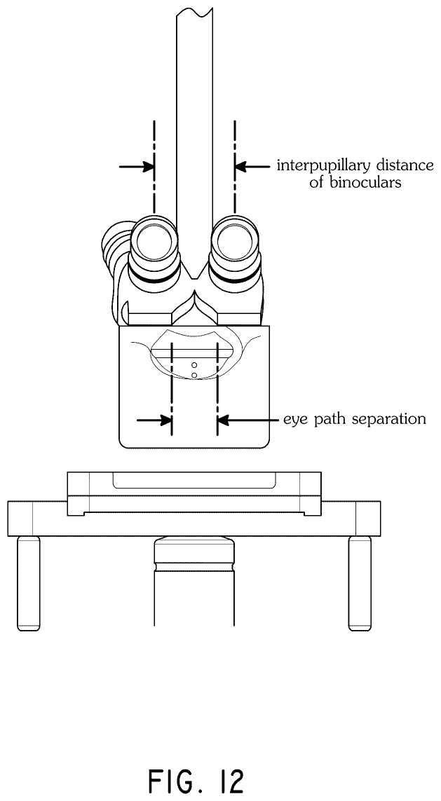

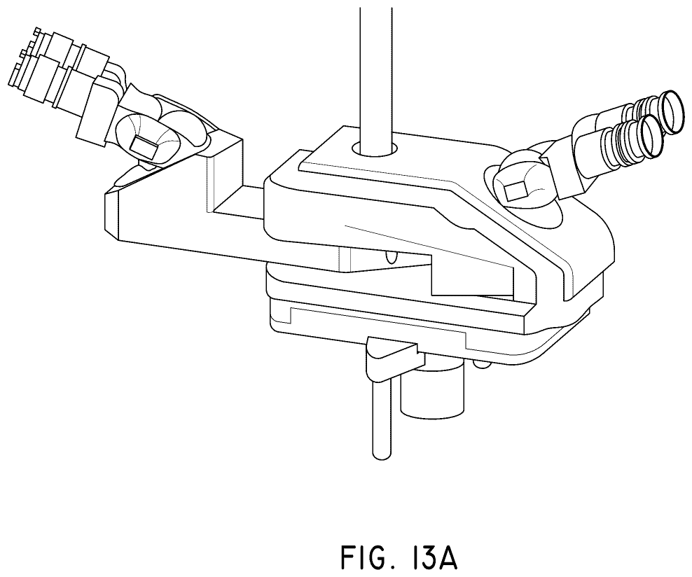

FIGS. 11A-11G illustrate example display optical systems 11300 configured to provide a view of a display 11310, the display optical system 11300 having an exit pupil 11305 wherein light paths that would intersect a viewing assembly housing 11315 are reduced or eliminated through baffles or apertures 11320, where a baffle includes a panel with an aperture. FIG. 11A illustrates an example embodiment of a display optical system 11300 comprising a display 11310, with other optical components configured to direct the light from the display 11310 to the exit pupil 11305. The light paths are traced with black lines to show the periphery of the bundle of light paths from the display 11310 to the exit pupil 11305. FIG. 11B shows this same display optical system 11300 as situated in an example viewing assembly housing 11315. When the display optical system 11300 is configured in this way, portions of the light 11320 from the display 11310 are outside of the housing 11315, which leads to light being reflected and/or scattered off the sidewalls of the housing along the path to the exit pupil 11305. This can lead to undesirable results, such as degradation in the quality of the image of the display 11310 viewed with an ocular, for example, by reducing contrast. The display optical systems 11300 can be configured to provide a collimated beam at the exit pupil 11305 such that a binocular viewing assembly comprising an objective and oculars can mate to the viewing assembly housing 11315 and view the display 11310.

In some embodiments, one or more baffles or apertures 11325 can be incorporated into the display optical system 11300 to reduce or eliminate the amount of light that intersects with the housing 11315. The apertures may be disposed to reduce the view of the sidewalls by the ocular, thereby reducing the light collected that is reflected off the sidewalls. FIG. 11C illustrates an example embodiment of the display optical system 11300 without any apertures. The display optical system includes mirrors M1, M2, M3, and M4 to redirect the light path within the viewing assembly. The mirrors M1, M2, M3, and M4 fold the optical path such that the display optical system 11300 can be contained in a more compact housing having a smaller footprint. Additionally in various embodiments, the mirrors M1, M2, M3, and M4 fold the optical path wraps around a supporting column configured to support the housing on an arm. In various embodiments the column is a conduit for electrical signals, power, and illumination fibers. Electronics boards, for example, with FPGAs, etc., can be disposed on the top of the display. Such a configuration may be useful because signal integrity (e.g. of MIPI2 signal) can be preserved with short cable routing. An opening 11307 for the support column about which the optical path is wrapped is visible in FIG. 11B. The display optical system includes lenses in lens tube L1 to shape (e.g., collimate) the light path along the path from the display 11310 to the exit pupil 11305. The lens tube L1 can be used to maintain a relatively narrow optical passageway that contains substantially all of the light travelling from the display 11310 to the exit pupil 11305. FIG. 11D illustrates the example embodiment of the display optical system 11300 from FIG. 11C with an aperture 11325 added between the mirror M4 and the display 11310. FIG. 11E illustrates the example embodiment of the display optical system 11300 from FIG. 11C with an aperture 11325 added between the mirror M3 and the final mirror M4. FIG. 11F illustrates the example embodiment of the display optical system 11300 from FIG. 11C with an aperture 11325 added between the lens tube L1 and the mirror M3.

FIG. 11G illustrates the example embodiment of the display optical system 11300 from FIG. 11C, with apertures 11325 added at all the locations illustrated in FIGS. 11D to 11F, between the lens tube L1 and the mirror M3, between the mirror M3 and the mirror M4, and between the mirror M4 and the display 11310. Simulations of the performance of this configuration have shown that the radiant intensity of unwanted light, e.g., light that arrives after being reflected or scattered from the inner housing of the housing 11315, have been reduced by about 3.6× while the radiant intensity at the exit pupil 11305 from the display 11310 has been substantially held constant which substantially means that there is less than a 10% change in the radiant intensity.

In some embodiments, the display optical system 11300 can include at least four baffles or less than or equal to four baffles. In certain implementations, four baffles can be included in the optical path between the first lens and the display 11310. In some implementations, two mirrors can be included in the optical path between the first lens and the display 11310. In some embodiments, the optical path can include, in order from the display 11310, a first baffle, a first mirror, a second baffle, a second mirror, and a third baffle prior to the first lens.

In some embodiments, the display can be a curved surface, for example either a projection display or recent generation of flexible LCD or OLED displays having high-resolution (e.g., in excess of 300 ppi). A curved display may provide two advantages: the imaging optics for the display can be less complex than for flat panels, and the cone or numerical aperture of each picture element in the display can be directed towards the viewing optics and in the periphery of the display, thereby providing a brighter image less subject to vignetting.

In some embodiments, the display can be a volumetric display comprising two or more transmissive display panels having a single backlight wherein the transmissive display panels are stacked to provide different planes of focus for a surgeon. The transmissive displays can be active matrix liquid crystal displays (“AMLCD”) or other types of transmissive displays. The backlight can be a fluorescent lamp, LEDs, or other suitable light source. By having displays positioned in different focal planes, image data from different focal planes may be presented to the surgeon with relatively less image processing and/or compression compared to a system which combines data from multiple focal planes into a single image. In some embodiments, a number of cameras can be positioned at varying depths or having varying focal distances such that the displays at different focal planes are configured to display image data from cameras positioned or focused at different depths to create a display that assists the surgeon in identifying positions of features within displayed images.

The display can show, as an overlay, pre-operative CT, MR, or other 3D image datasets from, for example, conventional surgical navigation systems (e.g., the Medtronic StealthStation or Treon, Stryker Surgical Navigation System, or Brainlab, among others). In various embodiments, in addition to images, the display can additionally provide numerical data and/or text. For example, in various embodiments, the display can overlay information such as distance or tool measurements, transparent tool renderings, camera identification information (e.g., the portion of the composite image attributable to a specific optical sensor may generate an identifying border around that portion), up/down orientation, elapsed time, and/or one or more still images captured from one or more optical sensors from a previous time in the operation The tracking system can provide 5-DOF (degrees of freedom) or 6-DOF position and orientation information to conventional surgical navigation systems. Other information, graphic, alpha numeric, or otherwise, can be provided.

The tool image can be magnified with respect to the wide-field view image, and change in image scaling will occur as the tool is moved in and out. In some embodiments, a visual metaphor for embodiments of the display is that of a hand-held magnifying glass for inspecting and doing work on a smaller region of a larger workpiece, while seeing the larger workpiece with lower magnification (if any) in more peripheral regions of the visual field to provide situational awareness. Tool images, for example, can be superimposed on the background image thereby blocking that portion of the background image. In various embodiments, the tool images may be stereo.

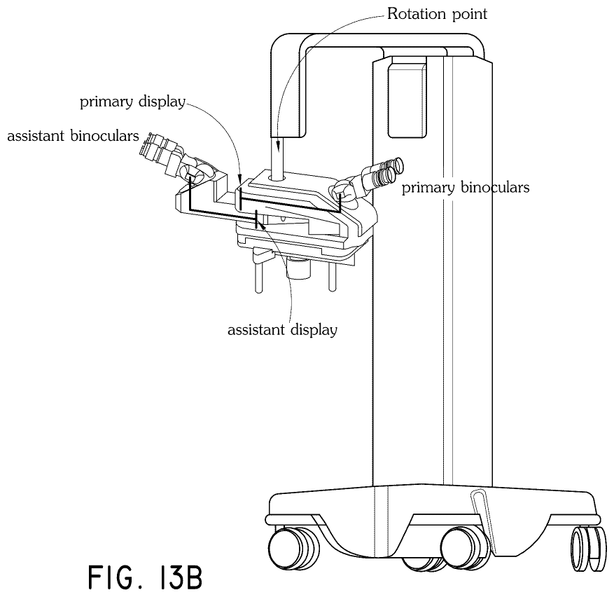

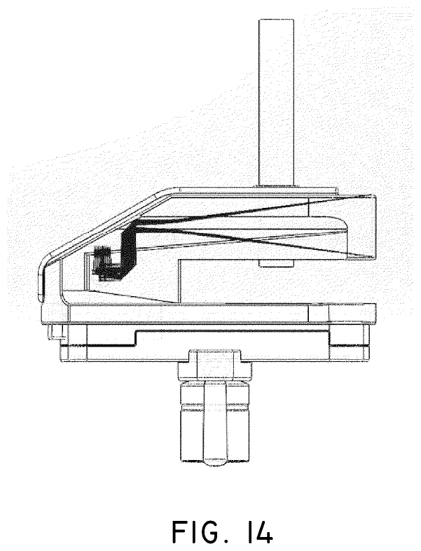

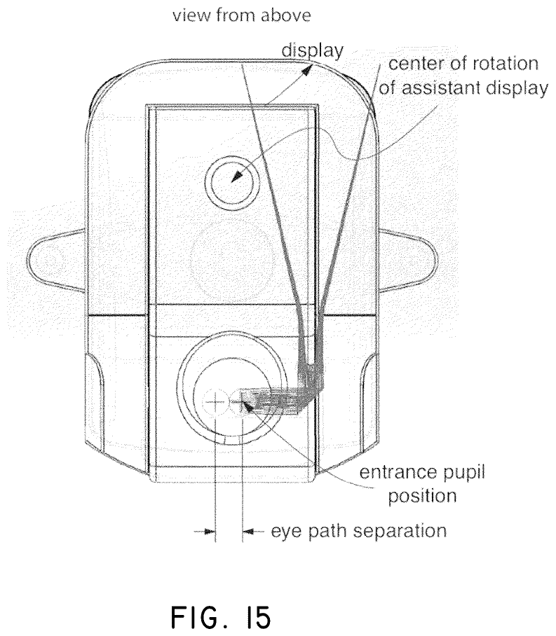

FIG. 12 shows a front view of a viewing platform comprising the binocular assembly and display enclosure assembly. Various embodiments optionally separate the two into self-contained components at a point in the optical path where the beam diameters of each eye path are approximately 18 to 20 mm in diameter where the beams may be collimated, and the separation between the 2 eye paths is approximately 22 to 25 mm. However, these separate optical portions or sections of the optical display system need not be on separate platforms or housings or otherwise be separately contained. The separation between eye paths is substantially less than that of the viewer, which is approximately 65 mm with a range of, for example, 52 to 78 mm. In some embodiments, the viewing platform can include binoculars for a primary surgeon and an assistant surgeon, as illustrated in FIGS. 13A and 13B. The collimated eye paths have a near constant diameter through a series of fold mirrors shown in FIGS. 14 and 15 and a first optical element. This compact folding with corresponding baffles within the section of the optical path comprises 40-50% of the total track of the optical path in the enclosure.

In various embodiments, the eye paths are mirror images of each other and are substantially parallel in direction, that is they have little or no convergence, and the display panels are orthogonal to the viewer in some embodiments, in plane with each other or parallel in other embodiments.

FIG. 15 shows a path from the exit pupil, which is outside of the enclosure, and matches within 25% the corresponding entrance pupil of the binocular assembly located in this same collimated section where the two components meet and attach. The overall optical path length of each eye from collimated pupil matching attachment point to electronic display for images is approximately 300 mm to 400 mm for the primary surgeon display. The ratio of path length to display diagonal is within a range of 1:2.2 to 1:3.5, display diagonal to path length. Various embodiments have display diagonals of 100 mm to 150 mm, one for each eye path.

The assistant surgeon display and display pathway are reduced in various embodiments to permit a more compact dual assembly. In the case of the assistant surgeon display being less than the primary surgeon display configuration in diagonal of viewed display and path length. The ratio of path length to display diagonal is within a range of 1:5 to 1:7, display diagonal to path length. Various embodiments have assistant display diagonals of 25 mm to 30 mm, one for each eye path.

With a near constant optical beam diameter of each path multiple mirrors are positioned between the first element group and the last element group with a single mirror in the converging beam path and the display. Between the last optical element and the display are 2, 3, or 4 or more baffles for stray light rejection. The aperture of each baffle is proportional to the display ratio, 3:4, or 16:9 or other.

When the surgeons are opposite one another, in some embodiments where the overlapping displays are nested within each other and where the assistant surgeon displays rotates about a point within the overlapping optical paths, the overlap ratio is 1:2 to 3:4.

The assistant surgeon display path may rotate around a rotation point through 270 degrees or thereabouts, the center of rotation taking the form of a column or bearing. Examples of primary and assistant displays with primary and assistant binoculars are illustrated in FIGS. 13A and 13B.

The rotation point of the assistant surgeon display is defined as a vertical column allowing the assistant surgeon eye pair to be horizontal through the range of rotation.

Various embodiments for the optical path of the primary surgeon display take the form of the following:

### Example 1

| | | | | |
|---|---|---|---|---|
| Surf | Radius | Thickness | Glass | Diameter |

Mirror (1^{st} turning)

| | | | |
|---|---|---|---|
| STOP | Infinity | 30 | 15 |

Mirror (2^{nd} turning)

| | | | | |
|---|---|---|---|---|
| 1 | 68.17 | 6 | N-FK5 | 25 |
| 2 | −282 | 3 | SF6 | 25 |
| 3 | Infinity | 106.48 | | 25 |
| | | (mirror) | | |
| 4 | 337.3 | 4 | N-BK7 | 25 |
| 5 | −186.75 | 2 | SF2 | 25 |
| 6 | −557.4 | 5.816 | | 25 |
| 7 | −43.14 | 3 | N-BK7 | 25 |
| 8 | −100 | 9.621 | | 25 |
| 9 | −33.96 | 2 | N-BAF10 | 25 |
| 10 | 32.51 | 4.5 | N-SF6HT | 25 |
| 11 | 189.23 | 187.897 | | 24.3 |
| | | (mirror) | | |
| 12 | Infinity | 0.01 | BK7 | 130.2 |
| Display | | | | 130.2 |
| | | | | (Diagonal) |

The input field of view, 2ω, is 6 degrees, but could be 3 degrees or as much as 10 degrees.

Various embodiments for the optical path of the assistant surgeon display take the form of the following:

### Example 2

| | | | | |
|---|---|---|---|---|
| Surf | Radius | Thickness | Glass | Diameter |

Mirror (1^{st} turning)

| | | | | |
|---|---|---|---|---|
| STO | Infinity | 35 | | 17.5 |
| 2 | −28.45 | 2.6 | N-SF6 | 28 |
| 3 | −37 | 2 | | 30 |
| 4 | −680 | 5.6 | N-BK7 | 30 |
| 5 | −43.29 | 50 | | 30 |
| 6 | 38.4 | 8 | N-SSK8 | 30 |
| 7 | 39.68 | 42.718 | | 28 |
| | | (mirror) | | |
| 8 | −30.7 | 4 | F2 | 28 |
| 9 | −43.29 | 48.93 | | 30 |
| | | (mirror) | | |
| Display | | | | 27.85 |
| | | | | (Diagonal) |

The input field of view, 2ω, (for example, of the electronic display e.g., LCD or LED display) is 6 degrees, but could be 3 degrees or as much as 10 degrees.

In the above embodiments, the relation between the input field angle, 2ω, and angle theta, the marginal field angle, on the display, the source, is shown below:

### Embodiment 1

2ω:6 degrees
2θ:30 degrees

### Embodiment 2

2ω:6 degrees
2θ:30 degrees

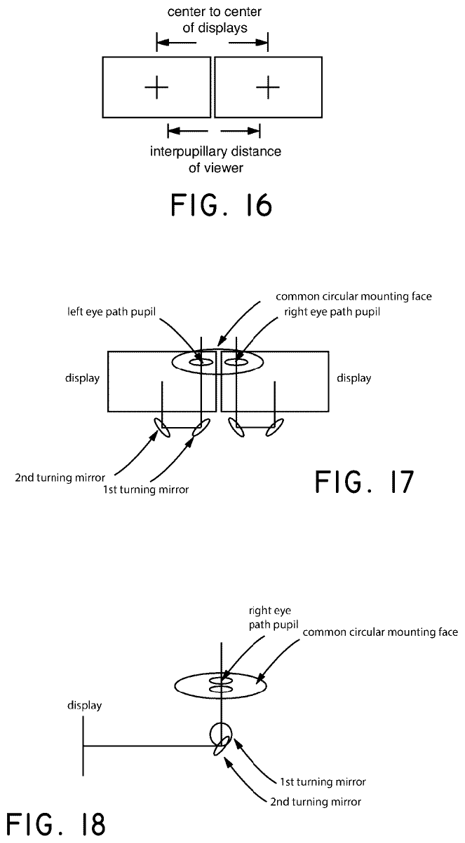

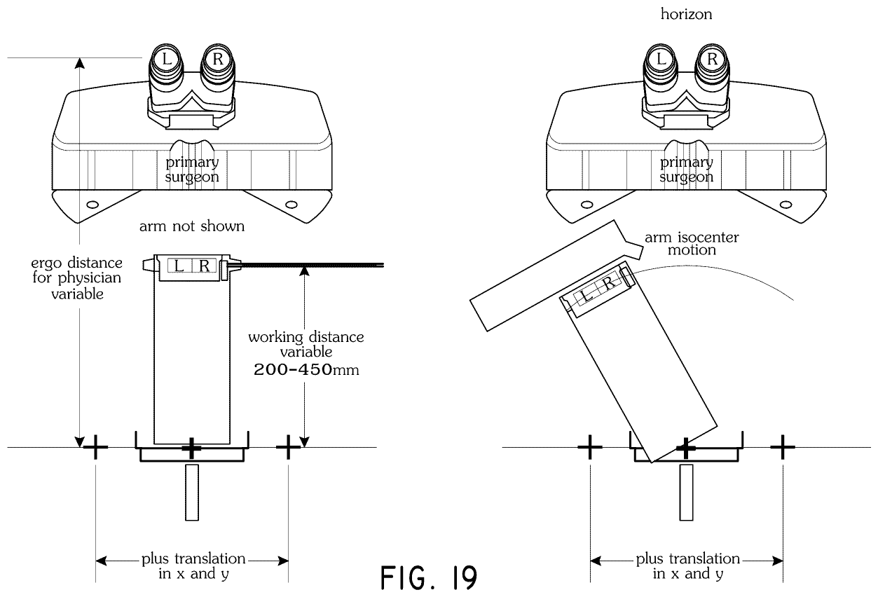

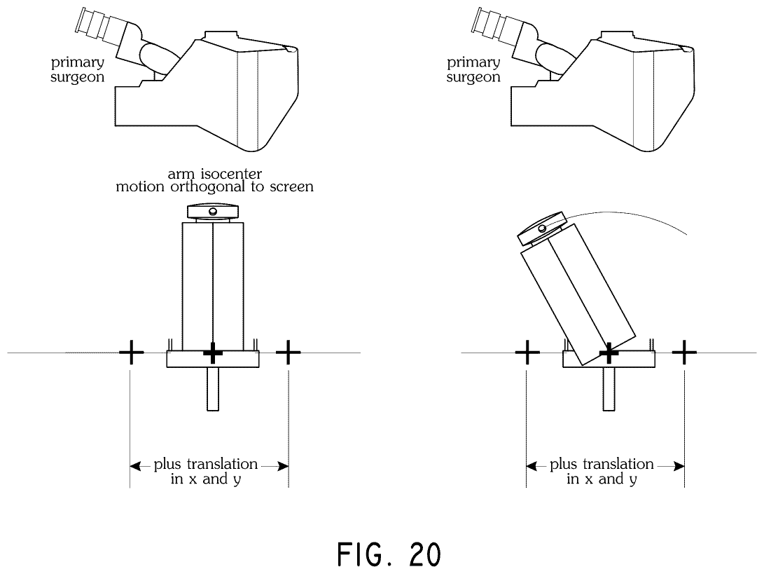

FIG. 16 shows the center-to-center distance of the displays being greater than the inter-pupillary distance of the surgeon. FIG. 17 shows the two eye path pupils and their respective center lines passing from a common mounting face that encircles both eye paths. This distance is less than the inter-pupillary distance of the user. FIG. 18 shows the first two turning mirrors, which adjust for the difference between the center-to-center distance of the displays and the inter-pupillary distance of the user in order to produce a compact electronic display with compact dimensions in axial and width dimensions for surgical imaging in 3D, stereo. The first turning mirror may reside in the space between the common mounting face and the first optical element in the prescription. Alternatively, the first turning mirror can be positioned after the first optical element. The second turning mirror resides within the spaces between the first and last optical elements of the prescription.

The optical system for each eye could have an equal sized display inserted in the path at the point of the last mirror which would be replaced by a beam splitter directing some portion of the path, likely 30, 50 or 70% of the energy to one path or the other. The second display may be used for fluorescence overlay or text or additional images.

Alternatively, the single display can be controlled at the CPU (central processing unit) level of overall system or by other processing and/or control electronics to provide a multitude of images optimized or configured for each (e.g., left and right) eye. Such optimizations/configurations can include real or calculated parallax. In some embodiments, fluorescence or different wavelength images or other images are superimposed on different images electronically.

The CPU or processing and/or control electronics can also select appropriate views from within the surgical site from any one of a number of sources based on the position of the assistant display using an encoder located, for example, on the rotation point or other known point. This can allow the assistant surgeon an appropriate view based on his or her position with respect to the primary surgeon.

### SOME EXAMPLE EMBODIMENTS

The following is a list of some example numbered embodiments. The examples presented herein are not intended to limit the scope of the disclosed embodiments, but merely represent exemplary combinations to illustrate potential uses and configurations. Nothing in the following should be interpreted to indicate that any one piece or component is essential to the embodiments disclosed herein.

1. A two-part (e.g. left and right) channel display or Wheatstone-like stereo display configured to provide left and right images with parallax provided by inter-pupillary distance between human eyes comprising a binocular assembly for the user and an electronic display assembly.
2. The display of Embodiment 1, where both assemblies are combined and the optical paths are collimated (the binoculars are focused at infinity).
3. The display of Embodiments 1 or 2, where each eye has a separate non-communicating path to one or more displays.
4. The display of any of Embodiments 1-3, where the entire binocular and display assembly is compact enough to fit over or near the patient (e.g., the doctor can reach the patient to operate while simultaneous viewing).
5. The display of any of Embodiments 1-4, where the binocular portion of the display can be ergonomically reconfigured while leaving the electronic display assembly stationary.
6. The display of any of Embodiments 1-5, where the exit pupil of the combined system is equal to or greater than the entrance pupil of the user's eyes.
7. The display of any of Embodiments 1-6, where the exit pupil of the electronic display portion is greater than or equal to the entrance pupil of the binocular assembly.
8. The display of any of Embodiments 1-7, where the inter-pupillary distance between the right eye path and left eye path is less than that of the user making the unit more compact.
9. The display of any of Embodiments 1-8, where the inter-pupillary distance between the right eye path and left eye path is approximately 22 to 24 mm at the exit face of the electronic display assembly and the entrance face of the binocular assembly.
10. The display of any of Embodiments 1-9, where two or more displays can be combined by beam splitters for each eye for the display of additional pre-operative and/or intra-operative information or add additional picture-in-picture like views or superimpose other wavebands in false colors over a first image in the other display.
11. The display of any of Embodiments 1-10, where two or more displays can be positioned and moved relative to a stationary beam splitter/beam combiner (e.g. 45 degree beamsplitter/beam combiner) to allow superpositioning of the displays or tile views of same.
12. A two-part Wheatstone-like stereo display comprising a binocular assembly for a second user and an electronic display assembly.
13. The display of Embodiment 12, comprising a second similar or smaller two-part Wheatstone-like stereo display as described in any of Embodiments 1-11 that can be integrally combined with a first display comprising any of Embodiments 1-11.
14. The display of Embodiment 13, where the second stereo display is used by an assisting surgeon.
15. The display of Embodiment 14, where the second stereo display can be repositioned around an axis so that the assisting surgeon can be positioned at 90 degrees to the first surgeon, or across the table from or 180 degrees with respect to, through to −90 degrees to the first surgeon.
16. The display of any of Embodiments 12-15, where repositioning the assistant stereo display does not change the position of the first surgeon's display.
17. The display of any of Embodiments 12-16, where the assistant surgeon's binocular portion can be ergonomically reconfigured without moving either system.
18. The display of any of Embodiments 12-17, where the assistant surgeon's views can be rebroadcast from that of the first surgeon display without modification or with modification such as mirroring or inverting.
19. The display of any of Embodiments 12-18, where the assistant surgeon's views can be modified by mirroring or rotating or other electronic processing as the assistant surgeon's display is rotated to a new position relative to the first surgeon.
20. The display of any of Embodiments 12-19, where the assistant surgeon's views can be different from the first surgeon's views permitting team surgery.

### Binocular Display Unit Incorporating Primary Surgeon Display and Assistant Display

As described above, electronic displays that receive video feed from stereo cameras can be provided for both the primary surgeon and assistant. Two separate binocular displays, one for the primary surgeon and one for the assistant can be used. Each display can have left and right electronic displays that present left and right channels of a stereo camera. Viewing the two displays together, which present images with the proper parallax for stereo imaging, the viewer can see 3D images (video) when peering through the oculars of the binocular display.

In certain embodiments, the two binocular displays can be attached and supported together on an articulating arm. In particular, a binocular display unit may comprise a separate primary surgeon display assembly and an assistant display assembly. Each can include a housing that includes therein a pair of electronic displays and imaging optics and possibly some folding and/or redirection mirrors. In various embodiments, each can also include a binocular assembly comprising a pair of objectives, prisms, and oculars for the left and right eye of the viewer.

In some embodiments, the primary surgeon display assembly and an assistant display assembly can be coupled together, for example, using a post or rotatable joint, and the assistant display assembly can rotate about the post or joint with respect to the primary surgeon display assembly. In this manner, an assistant standing opposite to the primary surgeon or standing to the left or right of the surgeon can look through the assistant display without moving the surgeon display assembly.

Accordingly in various embodiments, the binocular display unit includes primary surgeon oculars and displays on a single unit possibly in separate housings associated with the primary surgeon display assembly. This applies for the assistant display as well. However, the displays and oculars need not be separately housed and can be attached to a common base housing. Alternatively, the primary surgeon oculars and assistant oculars can be separable from the binocular display unit. When joined, the oculars and binocular display unit can form a single assembly. As another example, the oculars and binocular viewing assembly can be a substantially unitary assembly, wherein the oculars are substantially permanently affixed to the binocular display unit. As another example, the oculars can be configured to be removed and attached to the binocular viewing assembly with relative ease so that different oculars can be swapped in and out of the binocular viewing assembly. As discussed above, the assistant oculars can be rotatable and/or repositionable, in direction, for example, ±90°, 180°, with respect to the primary surgeon's oculars without moving the primary surgeon display. The assistant oculars and display assembly can be rotatable through intermediate angles for example from 90° through 270° (e.g., −90° to 90° or the primary and assistant oculars form an angle that is greater than about) 90° or more with respect to the surgeon and surgeon display assembly. Similarly, the primary surgeon oculars can be configured to rotate or otherwise be reoriented relative to the assistant oculars without moving the assistant oculars.

In various embodiments, the binocular displays can receive images from a plurality cameras including possibly left and right cameras on a stereo image pair. Depending on the position of the assistant, the selection of cameras may be different as well as presentation (e.g., orientation of the video images) may vary. Accordingly, in various embodiments, information regarding the location of the assistant is received and based on that location, processing electronics selects certain cameras to provide video images to the respective electronic displays in the assistant display assembly and orients or presents those images appropriately. For example, the images provided the primary surgeon may be mirror images of the images provided to the assistant if the assistant is standing opposite (facing 180° with respect to) the primary surgeon. Accordingly, manipulation (e.g., rotation) and/or selection of images may be varied depending on the location of the assistant. In certain embodiments, the location of the assistant can be determined based on the rotation of the assistant display assembly. Sensors such as encoders in the binocular display unit may provide such information. Other approaches to determining and/or monitoring the assistant's location including tracking sensors on the assistant (and/or primary surgeon) may be employed.

### Presentation of Combined 3D Image Content and 2D Image Content

As discussed above, in various embodiments images with parallax can be provided to left and right electronic displays that are viewed through respective left and right imaging optics and oculars. As a result, a surgeon or assistant viewing these displays through the pair of oculars will see a three dimensional image. In some embodiments images without parallax may be displayed on both the left and right electronic displays to produce a two-dimensional image. In some embodiments, the left and right displays can include both image content that includes parallax and image content without parallax. This image content could come from stereo and mono cameras, etc. (Stereo cameras have two channels, left and right, oriented at appropriate angles with respect to each other as the left and right eyes of a human to provide the appropriate parallax for three-dimensional rendition and perception.)

In various embodiments when both parallax image content and images without parallax content (corresponding to 3D image content and 2D image content, respectively) are provided, the images with parallax (3D image content) may be emphasized over the images without parallax (2D image content). For example, the 3D content may have brighter intensity and/or higher saturation than the 2D image content. Other parameters may also be used to emphasize the 3D content.

When 3D image content is visible to the viewer, stronger 2D image content may be distracting or degrade the viewing experience. Accordingly, emphasizing to 3D image content may be advantageous in some cases.

### Calibration of 3D Space

In various embodiments, calibrating the 3D space being imaged may be useful. In some embodiments for example, a calibration pattern may be projected onto the surgical site. For example, a light source and imaging optics may be employed to project an image of a reticle or other calibration pattern having known features and dimensions onto the surgical site being imaged, for example, by a camera configured to produce a surgical microscope view or a camera on a retractor or surgical tool. One or more of these cameras may image the projected calibration pattern together with the surgical site. Knowledge about the reticle pattern and how that pattern appears when projected onto a three dimensional surface will provide depth information about that three-dimensional surface. This information can be determined by processing electronics. In some embodiments a 3D CAD rendition of the surgical site may be generated based on the image of the surgical site and the information from the projected calibration pattern. Alternatively or in addition, measurements such as distance from one feature in the surgical site to another or the size of features in the surgical site may be determined. Volumes may also potentially be able to be calculated. The information may have other uses as well.

### Example Camera/Sensor Designs

Stereo cameras can be included on various medical devices, for example, retractors, surgical tools etc.). Such cameras can include a single sensor or detector array (e.g., CMOS, CCD, etc.) or a pair of sensors to obtain left and right eye views. A single sensor, for example, may be employed to obtain left and right images of a stereo camera pair. The sensor may be partitioned into areas to receive light from left and right imaging optics that produces left and right images on the active area of the sensor. A mask can be employed to partition the active area of the sensor into these left and right areas for receiving the left and right images. In some embodiments, stereo optics with left and right lens trains image onto the single sensor that is coupled to a processor configured to collect left and right images from the sensor at far left and right edges of sensor and superimpose those images to form a stereo image with same convergence as eye. See, for example, U.S. patent application Ser. No. 14/491,935 filed Sep. 19, 2014 which is incorporated herein by reference in its entirety, which shows stereo camera designs including single sensor and multiple sensor camera designs. The mask can be moved to collect light from different parts of the sensor. In some embodiments, the mask can be moved dynamically to accommodate variable optical parameters of the camera optics, for example, variable focus and working distance, which coincides with varying divergence. The mask may be implemented via software and corresponds to which pixels of the sensor to exclude from image formation. Conversely, the software implemented mask determines what pixels are used to collect image data. Separate left and right open portions of the mask where light to form the image is collected can be spaced farther apart or closer together depending on the desired convergence angle, focus, work distance, etc. Such dynamic masks can be employed in stereo cameras employing separate left and right optical sensors to account for different convergence angle, focus, work distance, etc. See, for example, U.S. patent application Ser. No. 14/491,935 filed Sep. 19, 2014 which is incorporated herein by reference in its entirety, which shows stereo camera designs including multiple sensors.

In other designs, it may be possible to have a chip with two spaced apart active regions thereon corresponding to left and right image channels. A single chip in a single package can comprise semiconductor and be patterned such that two spaced apart regions of pixels may be created to receive light from left and right lens trains. The space may include in some embodiments electronics or dead space. The space between these regions may not be active areas for collecting and sensing light. The spacing may accommodate for example the space needed for the two (left and right) lens trains or other physical components. A single 45° turning prism or a pair of 45° turning prisms may be employed to redirect light from said lens trains onto the front face and active regions of the sensor.

### Example Image Selection Control and Interface Designs

In various embodiments the surgeon may select images for viewing without lifting his or her head from the binocular display unit and/or without using his or her hands which may be preoccupied with surgical tools. Accordingly, in various embodiments, the images to be viewed are selected using an actuator (e.g., input device) that is not actuated by a hand. In some embodiments, the images to be viewed are selected by a user using a user interface element on a tool, such as a surgical tool. This can be done using the hand or by some other method of interacting with the user interface element. In some embodiments, a foot actuation device such as a foot pedal is employed. Accordingly, the surgeon can depress the foot pedal to indicate which video image feed or camera for example from the various cameras on the retractors and/or surgical tools or one or more cameras that provide a surgical microscope field of view the surgeon is interested in using. In some embodiment, the surgeon depresses a foot pedal to cycle through different video feed (e.g., thumbnails). The surgeon could depress another foot pedal to select one of these for enlarging etc. In certain embodiments, a single pedal may be employed. Depressing the pedal quickly will enable the surgeon to cycle through the various video feeds. Holding the foot pedal down a longer period of time may be used to select one of the video feeds for enlarging and/or placing the image at a particular location such as more central. Two or more foot pedals could also be used.

In certain embodiments, a display shows a single central image plus a plurality (e.g., 4) thumbnails disposed about a central image. Selection of which thumbnail replaces the image at center is specified by clicking on the input device, 1, 2, 3, or 4 times (e.g., if 4 thumbnails). The thumbnails can be arranged about the central image(s) like the numbers on a clock and the number of clicks can similarly be assigned to the thumbnail. For example, two clicks can designate the second thumbnail which is located in the lower right hand corner of central image. The central image can be enlarged in some embodiments. More than a single image may be included in the center. The selected image may be viewed at locations other than the center of the display. For example, two images, one main and another PIP, could be used. Other arrangements are possible.

### Cameras Configuration Providing Surgical Microscope Views

In various embodiments one or more cameras may provide surgical microscope views. In certain embodiments, a camera or stereo camera pair is disposed on the binocular display assembly as illustrated in FIG. 19. Such systems offer ergonomic benefits. For example, the angle of the camera or stereo camera that provides the surgical microscope view can be tilted to provide for a different view or perspective, yet the surgeon can remain in the same position. As shown, the oculars remain in the same horizontal plane despite the tilting of the camera. This feature can enable the surgeon to remain in a more comfortable position instead of contorting his or her body for an extended period of time to obtain a particular view of the surgical site during a surgery. This benefit is particularly useful for long physically arduous surgeries. Configurations that provide isocentered positioning, as shown, can reduce disorientation in some cases.

Lateral translation of the camera may also be possible without repositioning the oculars. Thus the surgeon can stay in the same position, which may be desirable.

FIG. 20 shows tilting in the orthogonal direction. Again the surgeon need not tilt his or her head to obtain views in this direction. As referred to above, configurations that provide isocentered positioning, as shown, can reduce disorientation in some cases.

Each of these views can be provided to the assistant through the assistant display as well. As discussed herein, in various embodiments the assistant display can be attached to the primary surgeon display in a binocular display unit.

Although shown on the binocular display unit, the camera providing surgical microscope views can be used on a platform and the binocular display unit so as to decouple the line of sight of the surgeon oculars from the camera further providing increased ergonomic benefits.

### Configurations for Temporal Approach

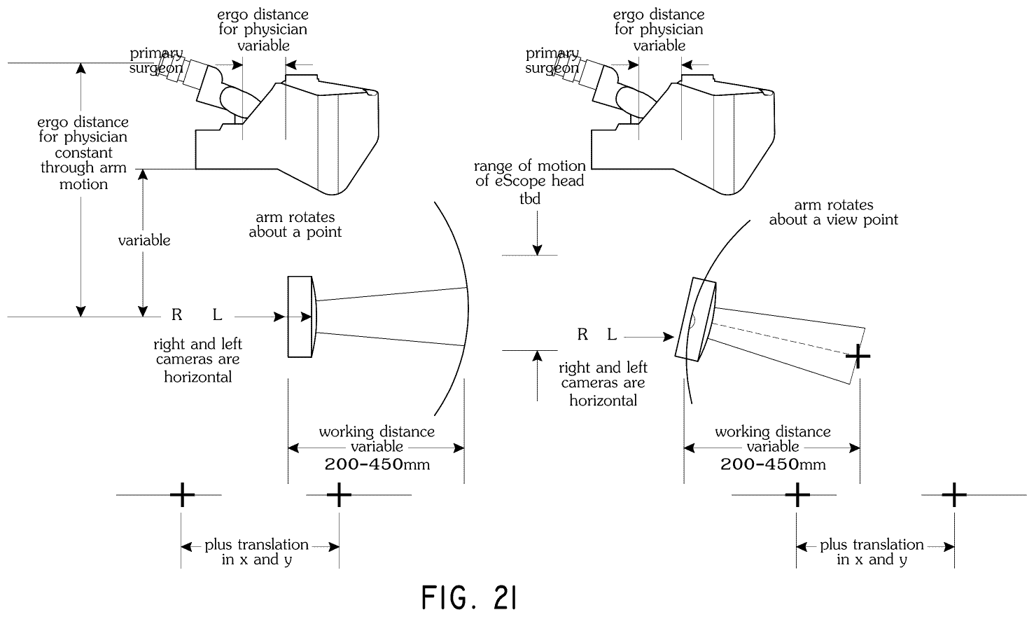

For some surgery, temporal access, for example, through the ear, through the side of the head or back etc. is desirable. Surgical microscope camera views from the temporal direction can be provided using the surgical microscope camera as shown in FIG. 21. A movement control system comprising a gimbal, for example, attached to the binocular display unit can support a camera the provides a surgical microscope view and that can be moved and reoriented to provide such oblique or temporal views. Once again, the surgeon need not tilt his or her head or re-position to obtain this perspective. Configurations that provide isocentered positioning, as shown, can reduce disorientation in some cases.

These views can also be provided to the assistant through the assistant display as well.

### Automatic Assistant Display Camera Selection and Image Manipulation

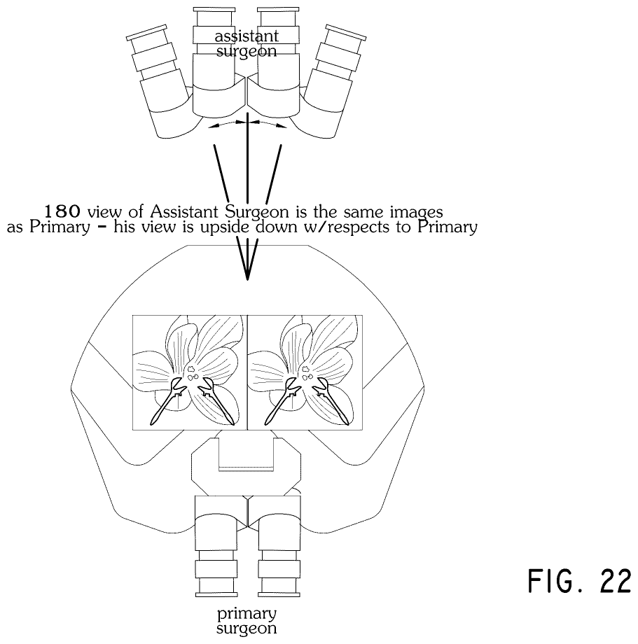

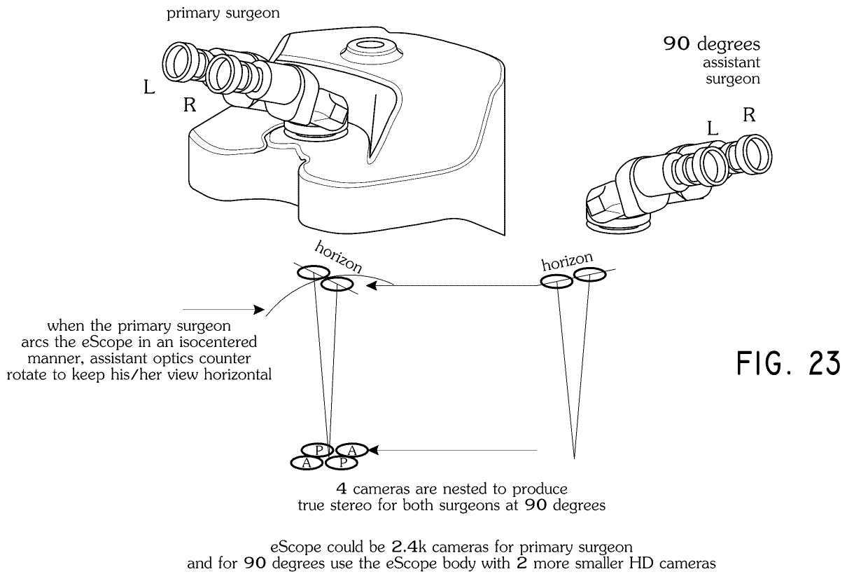

As discussed above, the assistant may be located opposite to the surgeon, or on the left or right of the surgeon, or in other locations. In various embodiments, the location of the assistant is sensed, and the appropriate camera views are provided to the assistant viewing the assistant electronic displays in the assistant binocular viewing assembly. FIG. 22 illustrates how the assistant opposite of the surgeon (with the assistant display assembly directed 180° with respect to the surgeon display assembly) is to see the images reoriented (e.g., upside down) and with the locations of the images reversed. Selection of cameras for different assistant positions, e.g., 180° position, 90° position, can apply to mono and/or stereo cameras on the retractor and/or on a surgical tool as well as to surgical microscope view cameras. Additionally, such camera selection based on sensing the assistant's location can be used for side/temporal (e.g., through ear) approaches discussed above.

### Variation in Assistant Location Accommodated by Multiple Cameras

In various embodiments, multiple cameras for providing a surgical microscope view may be employed to provide for the variation in assistant location. FIG. 23, for example, shows how four cameras nested in a 2×2 array can provide for surgeon view as well as assistant view where assistant is on the right side of the surgeon viewing form an orthogonal direction (e.g., 90°).

Accordingly, various embodiments include multiple cameras and sensors to accommodate multiple assistant locations (e.g., +90°, 180°). A processor can electronically provide the assistant display with the right/left mirror image of the surgeon's 3D camera pair for the 180° assistant position. Two stereo camera pairs oriented 90° with respect to each other effectively provides four nested camera fields arranged in a 2×2 array (e.g., a diamond), two orthogonal stereo pairs for surgeon and assistant, respectively.

In various embodiments, one of the sensors pairs can be a 4K sensor with higher resolution than the other sensor pair(s). For example, one sensor pair (or sensor with left and right portions for respective left and right eye views) can comprise a 4K, higher resolution sensor for the surgeon.

In some embodiments, a camera for the surgeon may be configured to move in an isocentered manner. In some embodiments the assistant optics is counter rotated to maintain the assistant display camera view horizontal.

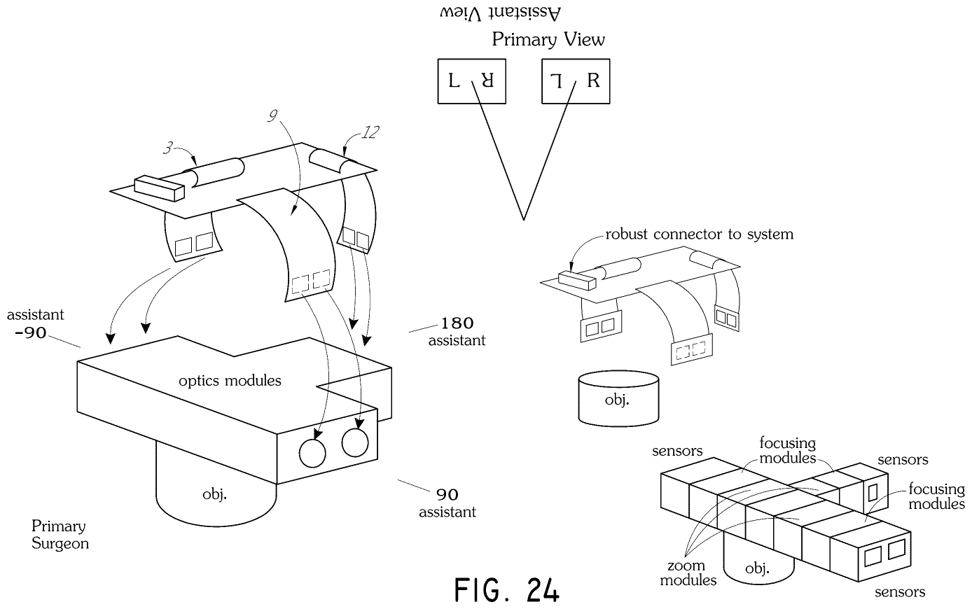

See also FIG. 24, which shows a system for providing multiple cameras providing surgical microscope views for the assistant. Note that in various embodiments, one of the stereo camera pairs at either the 3 o'clock position or the 9 o'clock position can be excluded. If, for example, the stereo camera pair for the 3 o'clock position is excluded, but the assistant is located at the 3 o'clock position, the stereo camera pair for the 9 o'clock position can be used with the images rotated upside down and the proper cameras selected for the left and right images that the assistant is to see.

FIG. 24 shows a common objective for the different sensors. A common objective may be employed for the sensors used for the primary surgeon as well as the sensors used for the assistant surgeon. A common objective may also be employed for left and right channels and left and right sensors (even if not used as a common objective for both the assistant and primary display). FIG. 23 show a common object employed for three sensor pairs, each having left and right sensors for respective left and right eye views.

### Methods of Surgery

In various embodiments a surgeon will use the camera that provides the surgical microscope view at the early stages of the surgery, for example, to make the incision for access into the body and to introduce tools initially into the body. As the tools progress into the surgical site, the surgeon may additionally use the cameras on the retractor. In certain cases, the surgeon will use the proximal retractor cameras initially and the distal retractor cameras thereafter as the surgical tool(s) passes deeper into the surgical site, for example, passing through proximal regions of the opening in the body into more distal regions into the surgical site. The various cameras can be employed to guide advancement of the tool into the desired depth in the body and into the surgical site. Similarly, with removal of the instruments, this process may be reversed (for example, the distal camera may be used more after relying on the proximal camera, and the surgical microscope camera may be used after the distal camera).

Various embodiments of the system may additionally be configured to provide for the same convergence angle for each of the stereo cameras, for example, the stereo camera that provides the surgical microscope view as well as stereo cameras on the retractor, including possibly both proximal and distal stereo cameras. Also, if a stereo camera is mounted on a surgical tool, such as for example, a Kerrison, this tool camera too may have the same convergence angle. In some embodiments, camera's having similar convergence angle can be selected as the tool progresses into (or out of) the surgical site and/or at different stages of the surgery. Having a similar convergence angle from one stereo camera to another should provide a more comfortable viewing experience for the surgeon.

The convergence angle is determined by the separation of the left and right cameras of a stereo camera pair that make up the stereo camera. These cameras obtain images of the object from different perspectives akin to the human's eyes separated by an inter-pupillary distance. The convergence angle is also determined by the distance to the object, for example, the working distance of the camera. In particular, the convergence angle depends on the ratio of the distance separating the left and right cameras and the working distance of the camera pair to the object.

The human brain and eye react to depth cues resulting at least in part from the convergence. Likewise images produced by stereo cameras having a convergence angle (based on inter-pupillary distance and working distance of the stereo camera pair), will provide depth cues to viewers of those images. As the surgeon may be transitioning between viewing images from the camera that produces surgical microscope views, the proximal and distal cameras on the retractor, and one or more cameras on surgical tools, the surgeon will receive depth cues from these different cameras. In various embodiments, the stereo cameras have the same convergence so as to avoid introducing changes among the depth cues as the surgeon moves from viewing video from one of the cameras to another and to yet another and back, for example.

In various embodiments, the camera that provides the surgical microscope view has a variable work distance. The surgeon may select a working distance for this camera that is suitable for the type of procedure to be performed. This work distance may establish a convergence angle, if for example the separation between the left and right cameras in the stereo camera pair is fixed. (In other embodiments, with variable convergence angle for the stereo camera that provides surgical microscope views, the surgeon may select a convergence angle.) In certain embodiments, other stereo cameras may be configured to be adjusted to also provide this same convergence angle. For example, the stereo camera or cameras on the retractor and/or surgical tool may be adjustable to provide the same convergence as is provided by the camera configured to provide surgical microscope views. Such cameras may include proximal and/or distal cameras on the retractor. See, for example, U.S. patent application Ser. No. 14/491,935 filed Sep. 19, 2014 which is incorporated herein by reference in its entirety, which shows stereo camera designs including single sensor and multiple sensor camera designs.

A mask associated with the two-dimensional detector array may be adjusted to provide for the desired convergence angle. For example, a single sensor may be employed to obtain left and right images of a stereo camera pair. See, for example, U.S. patent application Ser. No. 14/491,935 filed Sep. 19, 2014 which is incorporated herein by reference in its entirety, which shows stereo camera designs including single sensor and multiple sensor camera designs. The sensor may be partitioned into areas to receive light from left and right imaging optics that produces left and right images on the active area of the sensor. A mask can be employed to partition the active area of the sensor into these left and right areas for receiving the left and right images. In some embodiments, stereo optics with left and right lens trains image onto the single sensor that is coupled to a processor configured to collect left and right images from the sensor at far left and right edges of sensor. Using left and right displays, the left and right images are provided to left and right eyes of the surgeon or assistant, whose brain forms a third stereo image therefrom. The separation of the left and right areas that receive the left and right images establishes the inter-pupillary distant that together with the working distance controls the convergence angle. Accordingly, the mask can be moved to collect light from different parts of the sensor potentially increasing or decreasing this inter-pupillary distance. Moreover, in some embodiments, the mask can be moved dynamically (increasing or decreasing this separation) to accommodate variable optical parameters of the camera optics, for example, convergence, as well as variable focus and working distance. The mask may be implemented via software and corresponds to which pixels of the sensor to exclude from image formation. Conversely, the software implemented mask determines what pixels are used to collect image data. Accordingly, separate left and right open portions of the mask where light to form the image is collected can be spaced farther apart or closer together depending on the desired convergence angle.

Such a mask need not be limited to embodiments such as those with a single sensor. Embodiments such that employ two detector array chips can also have one or more masks that can be moved to accommodate for different optical parameters including convergence, work distance, focal length, etc. See, for example, U.S. patent application Ser. No. 14/491,935 filed Sep. 19, 2014 which is incorporated herein by reference in its entirety, which shows stereo camera designs including single sensor and multiple sensor camera designs. One or both two dimensional detector arrays can have masks having open regions that are laterally translated to change the distance separating the locations where light is collected, thus changing, for example, the convergence angle. As discussed above, the mask may be implemented via software and corresponds to which pixels of the sensor to exclude from image formation. Conversely, the software implemented mask determines what pixels are used to collect image data. Separate left and right open portions of the mask where light to form the image on separate respective chips is collected can be spaced farther apart or closer together depending on the desired convergence angle. In some embodiments, a mask is disposed on one chip while the other chip does not have such a dynamically moveable mask. By moving the mask on the one chip, however, optical parameters such as convergence can be altered. For example, if the chip on the left has a dynamic mask, the open portions in the mask can be spaced farther apart or closer to the chip on the right depending on the desired convergence angle.

Accordingly, the mask can be adjusted, for example, one or more openings therein can be translated, to provide for the same convergence between stereo cameras on the retractor and/or surgical tool as on the stereo camera that provides surgical microscope views. For example, for a particular surgical procedure, a convergence angle may be initially established by selecting a working distance for the stereo camera providing the surgical microscope view depending on the type of procedure to be performed. This selection of working distance may establish a convergence angle between the left and right channels of the stereo camera pair that provides surgical microscope views. A mask on one or more other stereo cameras (e.g., a stereo camera pair on a surgical tool, proximal and/or distal stereo cameras on a retractor, etc.) may be changed or reconfigured, for example, by moving one or more openings therein, to provide the same convergence angle as provided by said one or more stereo camera pairs. Consequently, using the reconfigurable mask with movable aperture(s), stereo camera pairs on retractors or surgical tools may be provided with a similar convergence as the stereo camera pair providing the surgical microscope view. By maintaining the same convergence for the different cameras, the depth cues provided the surgeon can be maintain relatively constant despite viewing images from different stereo cameras (e.g., surgical microscope view camera, proximal retractor camera, distal retractor camera, surgical tool camera, etc.). As a result, a more comfortable viewing experience may be provided.

In certain embodiments, the stereo camera may additionally provide adjustable focus. One or more actuators may be included that are configured to translate one or more lenses in the camera optics that images the surgical site onto the two-dimensional detector array to change the focus of the camera. These actuators may be driven electrically in some embodiments although different types of actuators could be employed. These actuators can be included in the package that supports the camera and is disposed on the retractor. Advantageously, cameras on retractors (in contrast for example to endoscopes) have available space lateral to the imaging lenses (e.g., in the radial direction) in which such actuation devices can be located. The result may be that the lateral dimensions (e.g., in x and y) exceed the longitudinal dimensions (z), however, surgical access to the surgical site would not be impeded by utilization of the space surrounding the lenses in the lateral or radial directions.

In various embodiments, when the focus is changed using the actuator, the mask may be reconfigured or changed as discussed above. For example, one or more open region or aperture in the mask through which light is directed to the left and/or right channel can be shifted laterally to increase or decrease the convergence angle. In this manner, the convergence angle of the stereo camera with the adjustable focus disposed on the retractor or surgical tool can be altered to be the same as the convergence angle of the stereo camera providing the surgical microscope views. Constant convergence angle for different stereo cameras can be provided even if such cameras include an adjustable focus. Both the focus and the mask can be changed as needed to provide the desired focus and convergence angle.

Incorporating an adjustable focus enables a camera lens having a smaller depth of focus to be employed. Such a camera lens will have a larger numerical aperture and smaller F-number than a similar lens that produces a larger depth of focus. Some benefits of the larger aperture lens are increased light collection and resolution.

In some embodiments, one or more lenses may be translated laterally to alter the convergence of the stereo camera. For example, one or more lens included in imaging optics for the left and/or right channel may be translated orthogonal to the optical axis or optical path to the sensor to alter the convergence angle, for example, to provide different or the same convergence angle with different work distances, focuses, similar to the use of the laterally displaced mask discussed above.

### Assistant Display

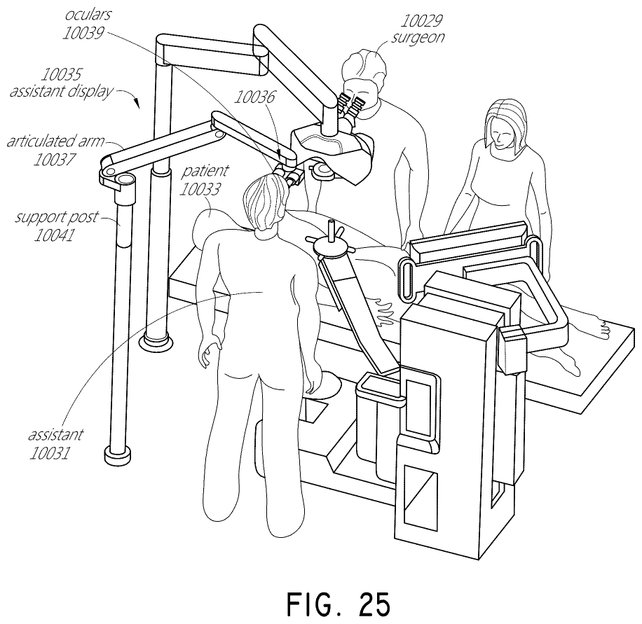

FIG. 25 is a schematic illustration of a surgical visualization system including an assistant display. In some embodiments, a separate assistant display may be provided for use by a surgical assistant or observer. As illustrated in FIG. 25, the assistant display 10035 comprises a binocular viewing platform 10036 for the assistant that includes oculars 10039 mounted on a lockable articulated arm 10037, which extends from a support post 10041. The assistant binocular viewing platform can include one or more displays such as LCD or organic LED displays as described with regard to the surgeon's viewing platform. Likewise, optics may also be included in the binocular viewing platform to provide a view of the display through the oculars. A Wheatstone set-up may for example be used in some embodiments. In various embodiments, the assistant display 10035 can include, for example, one or more eMagin NTE AMOLED displays. In some embodiments, the display can provide a three-dimensional view, as described in more detail above with respect to the surgeon binocular display. In some embodiments, the viewing platform may be disposed above and/or over the patient, similar to a surgical microscope. The viewing platform may be disposed on an articulated arm so as to be arranged above and/or over the patient, similar to a surgical microscope. Providing the viewing platform above or over the patient permits the surgeon to be sufficiently close to the patient (e.g., at the patient's side) to perform the surgery while looking through the oculars. Additionally, the viewing platform is compact, thus allowing the surgeon to be in close proximity to the patient without being separated from the patient by a bulky system. The oculars can thus be disposed sufficiently over the patient so that the surgeon's hands can reach the patient to perform surgery. Advantageously, the surgeon's close proximity to the patient can allow the surgeon to more closely monitor the patient during the surgery.

Accordingly, in various embodiments described herein, a surgical visualization system includes a viewing platform, for surgeons and/or assistants etc., comprising a housing containing one or more displays therein. The displays provide video from a camera viewing the surgical site. In various embodiments, the viewing platform does not provide a direct view through the housing. The surgeon or assistant does not see through the housing directly viewing the surgical sight using light passing from the surgical site through the housing. Instead, the viewer peers into the housing, via oculars, at displays. The displays present images obtained from cameras sensors viewing the surgical sight. Such a configuration provides ergonomic benefits as the line of sight of the stereo camera is decoupled from the displays and/or oculars so that the surgeon or assistant need not have his or her line of sight aligned with the line of sight of the stereo camera providing the surgical microscope view. This configuration and benefit may apply to both the surgeon and assistant displays. Mirrors may be employed to direct left and right images from one or more displays to left and right oculars. In some embodiments left and right displays or left and right eye portions of a display provide images with parallax consistent to that of human eyes or Wheatstone configuration is used to provide stereo. Mirrors may fold the optical path from the display(s) to the oculars thereby providing for a more compact smaller footprint. Additionally, lenses may be employed in the optical path from the display(s) to the oculars. The lenses may collimate the light from the display and form a collimated or substantially collimated beam that is received by the oculars. The lenses may also reduce the beam size. For example, one or more rectangular displays having a diagonal between 3-8 inches, 4-6 inches, e.g., 5 inches may be used. The lens may collect and collimate light from such a large object and reduce the beam to a smaller size for the oculars which may have an aperture size, for example, between about 0.3-2.0 inches or 0.5 to 1.0 or 1.5 inches. Producing a beam having a reduced cross section enables smaller folding mirrors to be employed. In various embodiments the housing is compact and has a small footprint. The viewing platform may provide three-dimensional images via the left and right pair of oculars. Accordingly, in various embodiments, the viewing platform has a similar feel as a surgical microscope that is a compact a stereo binocular microscope, to be familiar to surgeons. The viewing platform may be disposed above and/or over the patient, similar to a surgical microscope. The viewing platform may be disposed on an articulated arm so as to be arranged above and/or over the patient, similar to a surgical microscope. Providing the viewing platform above or over the patient permits the surgeon to be sufficiently close to the patient (e.g., at the patient's side) to perform the surgery while looking through the oculars. By providing a compact viewing platform that can be disposed proximal to, above and/or over the patient allows the surgeon to be in close proximity to the patient without being separated from the patient by a bulky system. The oculars can thus be disposed sufficiently over the patient so that the surgeon's hands can reach the patient to perform surgery. Advantageously, the surgeon's close proximity to the patient can allow the surgeon to more closely monitor the patient during the surgery.

### Example Optical Configurations for a Surgical Microscope View Camera Having a Common Objective Lens

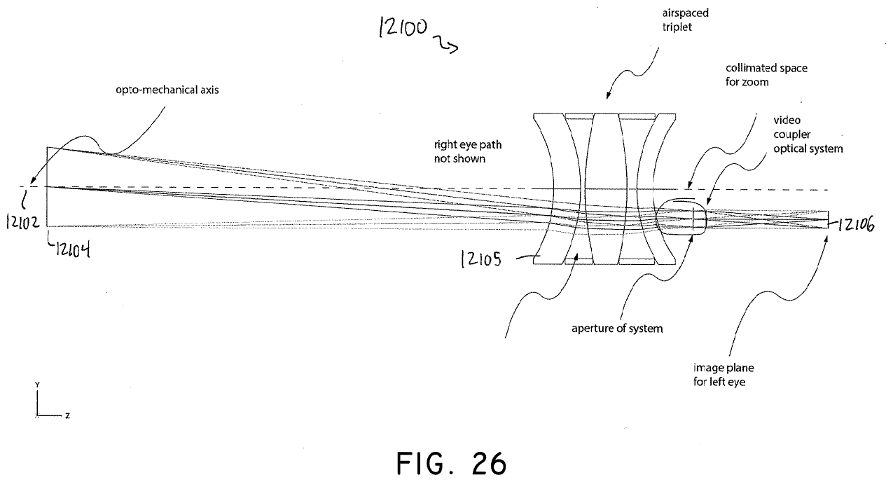

FIG. 26 illustrates an example imaging system 12100 comprising an objective triplet 12105. The objective 12105 has an opto-mechanical axis 12102 that runs through a center thereof. The illustrated imaging system 12100 shows only the optical path for the left eye imager, but it is to be understood that a similar image would be formed for a right eye imager along a right eye optical path. The objective triplet 12105 comprises an air-spaced triplet lens configured to collimate light for a zoom system and/or a video coupler optical system, examples of which are described herein. The aperture of the system can be located after the objective triplet 12105 (e.g., rearward the objective triplet, distal from the surgical site, etc.). An image of object 12102 is formed along the left eye optical path at the left eye image plane 12106. An image sensor here can then produce video for a left-eye view in a binocular display system, as described herein. A counterpart right-eye view image (not shown) could also be produced on the opposite side of the opto-mechanical axis of the objective at a similar longitudinal position along the length of the triplet opto-mechanical axis as the left eye image. In some embodiments, the air-spaced triplet 12105 can be a super achromat.

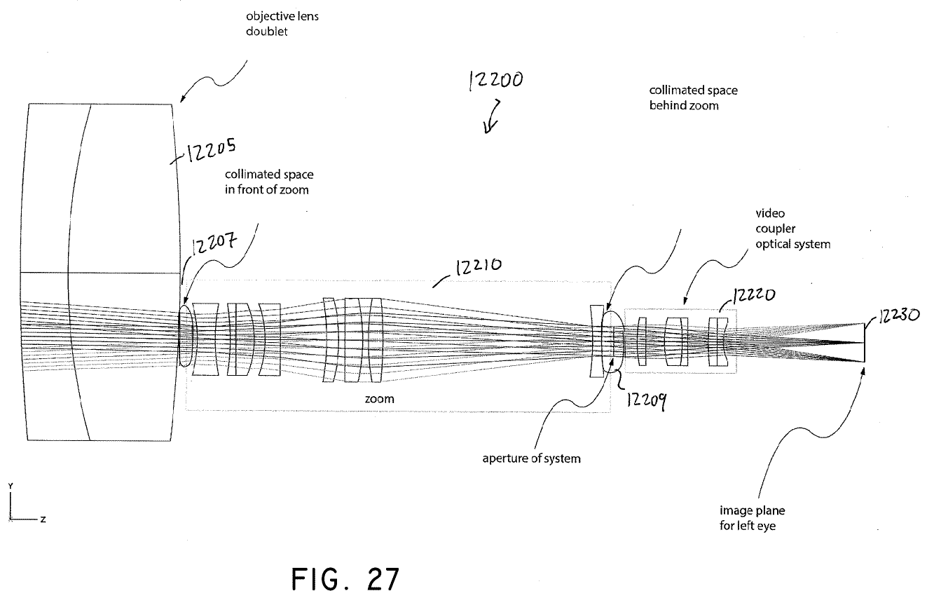

FIG. 27 illustrates an example imaging system 12200 comprising a common objective lens 12205 for both optical paths of stereo imagers. The imaging system 12200 can be configured to provide a relatively high zoom factor. The common objective lens 12205 can be a doublet used at its focal length. The doublet can be an achromat. In some embodiments, the common objective lens 12205 can comprise more than two lens elements, e.g., three, four, five, or more than five lens elements.

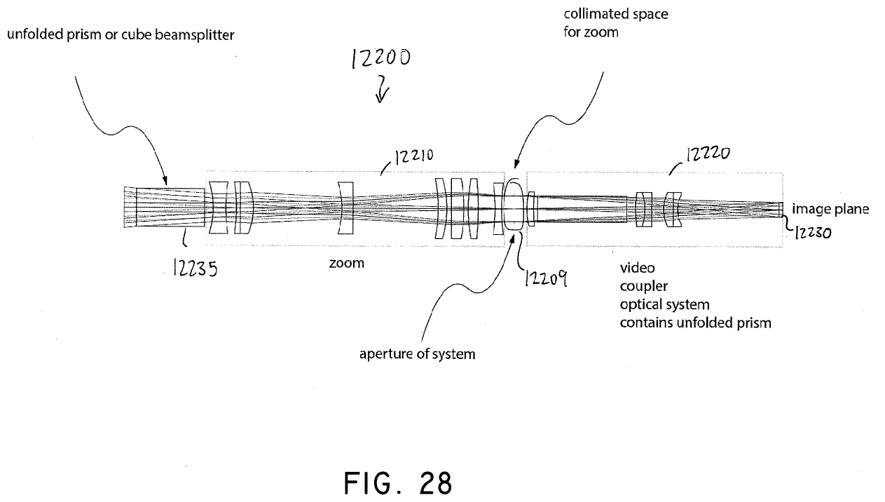

In various embodiments, the afocal lens group may comprise first and second lenses or lens groups lens separated by a distance. An additional lens or lenses may be included, for example, between these two lenses. In the embodiment shown, for example, a central lens group is shown disposed between first and second power lens groups. In some embodiments, such as shown in FIG. 27, the first and second lens or lens groups are negative and the central lens is positive. In contrast, however, as shown in FIG. 28, the first and second lens or lens groups may be positive and the central lens is negative. As shown in FIGS. 27 and 28, however, these first and second lens groups and the central lens group may comprise positive and negative lenses or just a single positive or negative lens. A variety of other configurations are possible. The lenses may be in different locations and separated by different distances. One or more lenses may be moved to change magnification. In various embodiments, the lens group is afocal such that collimated light input by the afocal zoom will be output as a collimated beam. Magnification may also be provided. In various embodiments, an afocal zoom such as shown in FIG. 27 is disposed in each of the right and left optical paths (e.g., corresponding to right and left eye views for a stereo display).

The light from an object can be collimated by the objective lens 12205 to produce collimated light output 12207. The collimated light 12207 can then enter the afocal zoom lens group 12210. The afocal lens group 12210 is configured to receive collimated light and output collimated light. As described above, the afocal lens group 12210 can include one or more moving lens elements and/or variable power optical elements to change a magnification of the collimated light output to result in a zoom lens system. Also, the collimated space 12207 between the objective lens 12205 and the zoom lens group 12210 can be of any arbitrary size (e.g., length along the optical path)

The collimated light exiting the afocal zoom lens group 12210 passes through an aperture 12209 before entering the video coupler optical system 12220. The focal length of the video coupler optical system 12220 can be configured to provide a suitable image size at an image plane 12230, wherein the suitable size can depend at least in part on the size of an image sensor at the image plane 12230. The video coupler optical system 12220 comprises a plurality of lens elements configured to receive collimated light and focus the light at the image plane 12230. In various embodiment, the video coupler optical system 12220 has positive optical power to focus the image on a sensor array at the image plane 12230. The video coupler optical system 12220 may provide color correction. Multiple lens element comprising different material with different Abbe number, for example, may provide color correction. For example, one or more doublets that provide color correction can be included such as shown in FIG. 27.

In some embodiments, the video coupler lens group can be configured to cause relatively small translations to result in relatively large changes in work distance. For example, the ratio of the change in lens translation to working distance may be from 1:10 to 1:40, 1:20 to 1:30. In one example, where the ratio is 1:25, the video coupler lens group can be configured to move in the longitudinal direction about 1 unit (e.g., 1 mm-2 mm) to result in a change in about 25 units (e.g., 25 mm-50 mm) in working distance. This can make it relatively easy to use one hand to adjust the working distance, relative to previous systems where it was difficult to adjust the working distance with one hand. Accordingly, the work distance can be moved optically by changing one or more lenses within the optical system and the acquisition system as a whole need not be moved, for example, if placed on a movable arm, the arm need not be moved.

In various embodiments, collimation of the beam output from objective 12205 permits a wide range of the longitudinal distances between the objective and the zoom system 12210. In certain embodiments, therefore, optical elements such as prisms may be included between the objective and the zoom system, because, in various embodiments, the beam will be collimated in this location. Other designs are also possible.

Similarly, in various embodiments, collimation of the beam output from the zoom system 12210 permits a wide range of the longitudinal distances between the zoom system and the video coupler optical system. Accordingly, in some embodiments, one or more prisms can be positioned in the collimated light space before or after the aperture 12209, between the afocal zoom lens group 12210 and the video coupler optical system 12220. In the embodiments shown, the aperture is placed between the zoom group and video coupler group. The prisms or other optical redirection elements can be configured to adjust the distance between the right and left eye image paths to accommodate the size and positions of image sensors for the respective images. For example, when the imagers are larger than inter-pupillary space, prisms can be used to offset right and left eye optical paths.

Although a zoom system 12210 and video coupler optical system 12220 for a left eye optical path are shown, a similar zoom system 12210 and video coupler optical system 12220 for a right eye optical path can also be included. FIG. 28 illustrates the zoom lens group 12210 and the video coupler optical system 12220 of the example imaging system 12200. In certain embodiments, the prism or cube beam splitter may be used to alter the direction of the beam and possibly make the camera system more compact or configured in a more desirable shape for the particular application. The objective, for example, can be oriented so as to point in one direction and the zoom and video coupler systems can be oriented in a different, for example, orthogonal direction.

As described above, the zoom systems 12210 shown in FIGS. 27 and 28 have different designs. Similarly, the video coupler optical systems 12220 also have different designs. For example, the video coupler optical system shown in FIG. 28 shows an unfolded prism between a first lens element and a second lens element. Such a prism may permit the optical path to be redirected, for example, to provide a more compact of desirable shape.

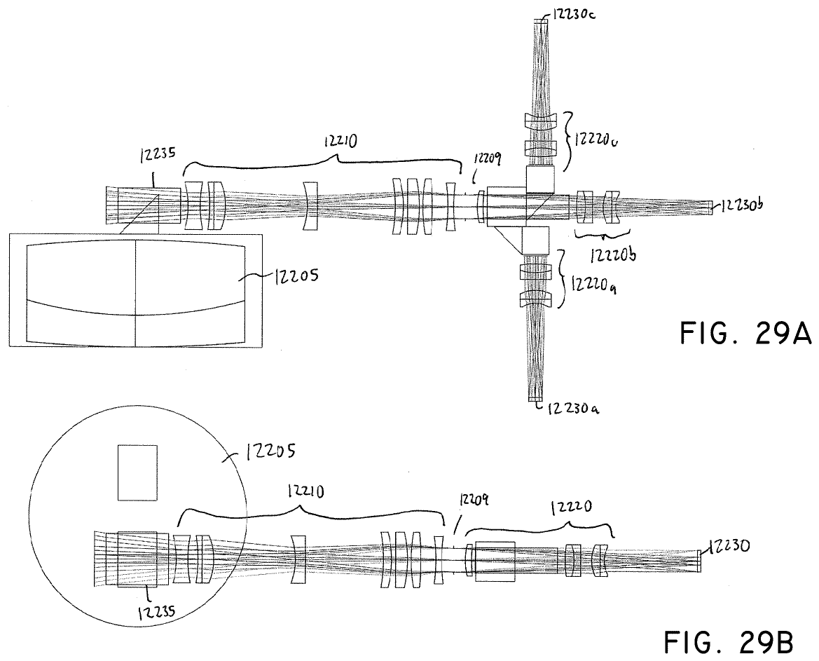

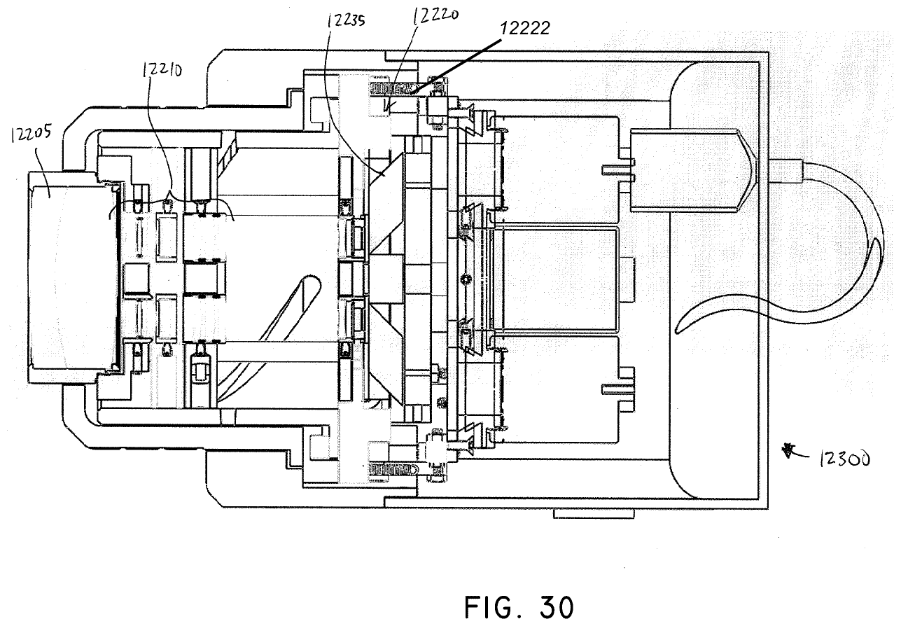

FIGS. 29A-29B illustrate respective side and top views of the example imaging system 12200 having a common objective lens 12205 for stereo imagers. As illustrated in FIG. 29A, a prism is included between the objective 12205 and the zoom system 12210 to permit the object to face in a first direction and the zoom system to extend in a longitudinal direction along a second perpendicular direction. Such a configuration may make the system more compact or reduce the footprint of the overall imaging system. FIG. 29A also illustrates three optical paths to three images plane (and respective optical sensor locations) 12230a,b,c. The different optical paths can be used for different purposes. For example, each optical path, in various embodiments, can be used for different (e.g., separate, adjacent, or overlapping) wavelength bands for imaging. For example, the optical paths can be used for infrared, visible, and/or near infrared image acquisition. Other wavelength ranges are possible as well. In some embodiments, one or more optical path can be used for stereo image acquisition and one or more optical paths can be used to acquire monocular images. In some embodiments, the optical paths can lead to image sensors with different resolution. In various embodiments, the stereo imagers and monocular imagers have different resolutions, wherein the monocular resolution exceeds the stereo resolution (e.g., the resolution of one of the pair of image sensors in the stereo pair). For example, a 4K sensor may be included at the image plane 12230b (with lower resolution sensors in other optical paths). An image from this 4K sensor may, for example, be displayed on a flat screen or panel display for multiple viewers. This image may alternatively be provided to the binocular display of the surgeon, e.g., possibly in mono. Other uses for these different paths are possible. Additional optical paths, for example for fluorescence imaging or other purposes are possible. Additional beam splitters or folding optics (e.g., mirrors) may be employed as needed.

In the design shown in FIG. 29A, as also shown in FIG. 28, the beam splitter or fold element(s) are included within the video coupler 12220 between the first and second optical elements.

FIG. 29B illustrated one of the left or right eye views from a top view. A counterpart right or left eye view optical system could also be included for stereo imaging.

The common objective 12205 collimates light that passes through the prism, fold element, or beam splitter 12235 to redirect the optical path. The collimated light enters the afocal zoom lens group 12210, which provides collimated light output. The aperture 12209 restricts the aperture of the imaging system 12200. The collimated light then passes through beam splitters and optical redirection elements 12240 to generate a plurality of optical paths. Each optical path comprises a video coupler optical system 12220a, 12220b, 12220c configured to generate an image at image planes 12230a, 12230b, 12230c.

FIG. 30 illustrates a cut-away view of an example stereo imager housing 12300 having a common objective 12205. The housing 12300 provides mechanical support for the imaging system 12200 comprising the objective 12205, the afocal zoom lens group 12210, the video coupler optical system 12220, and image sensors 12222.

In some embodiments fluorescence images can be collected and displayed. These fluorescence images may be viewed superimposed on images of the surgical site not based on fluorescence. Cameras that image in different wavelengths, such as infrared, could image the surgical site or objects contained therein. In some embodiments, features could be made to fluoresce, for example, by injecting fluorescent chemical and illuminating the area with light that will induce fluorescence. For example, in certain embodiments anatomical features may contain fluorescent dye that fluoresces, for example, when exposed to short wavelength radiation such as UV radiation. Such a technique may be useful to identify and/or highlight the location and/or boundaries of specific features of interest such as tumors, etc. The fluorescence or other wavelength of interest may be detected by the one or more cameras imaging the surgical field such as one or more camera providing a surgical microscope view. For example, an optical detector that is sensitive to the wavelength of the fluorescent emission may be employed to view the fluorescent image. In some embodiments, the wavelength of fluorescent emission is in the infrared. In certain embodiments sensors sensitive to different wavelengths may be employed. In particular, one or more sensors sensitive to the fluorescing wavelength (e.g., IR) may be used in conjunction with one or more sensors not sensitive or less sensitive to the fluorescing wavelength but sensitive or more sensitive to other useful wavelengths (e.g. visible light). Light can be collected and distributed to both types of detectors for example using a beam splitter such as a wavelength dependent beam splitter that reflects one wavelength and passes another. The fluorescent and non-fluorescent images can be recorded by the respective sensors. In some embodiments, the fluorescent and non-fluorescent images can be superimposed when displayed on electronic displays that receive image data from both types of sensors.

In some embodiments, images produced by fluorescence or other wavelengths of interest are superimposed on one or more images from other camera(s). Filtering could be provided to remove unwanted wavelengths and possibly increase contrast. For example, the filter can be used to remove excitation illumination. In some embodiments emission image content, (e.g., fluorescing tissue) can be parsed and superimposed on image content that is not emitting (e.g., tissue that is not fluorescing), or vice versa.

In some embodiments, IR fluorescence images are superimposed over non-IR (e.g. visible) images. Other wavelengths such as other fluorescence wavelengths may be employed. In various embodiments, such as where the fluorescing wavelength is not visible (e.g., for fluorescence in the infrared), an artificial color rendition of the fluorescing content can be used in place of the actual fluorescing color so as to enable the fluorescing tissue to be visible.

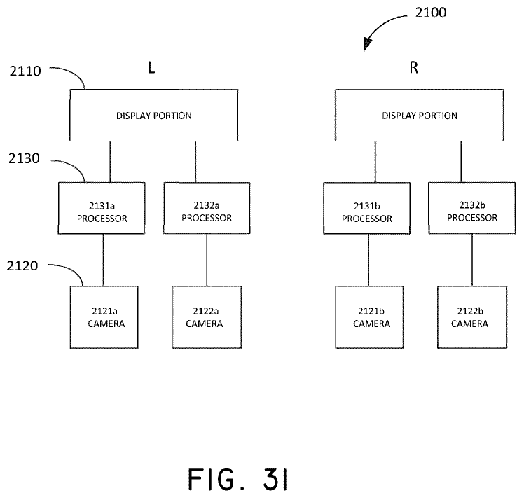

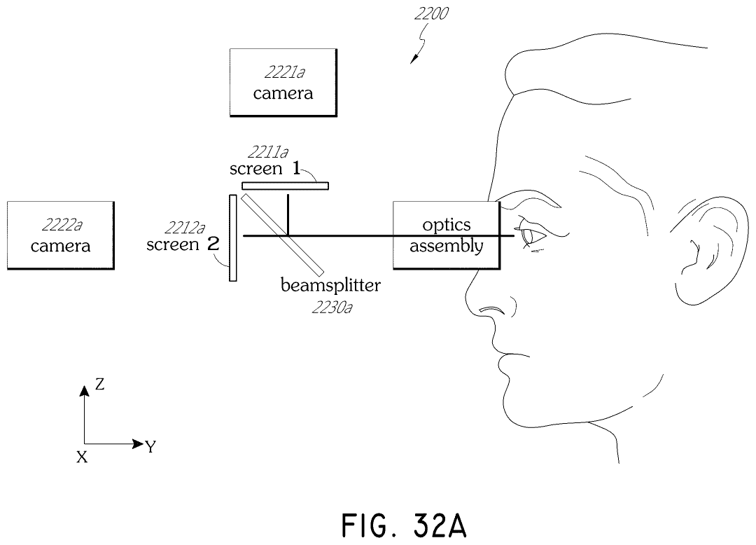

FIG. 31 schematically illustrates an example medical apparatus in accordance with certain embodiments described herein. The medical apparatus 2100 can comprise a display (or display portion) 2110, a plurality of cameras 2120, and one or more processors 2130. The plurality of cameras 2120 can include at least one first camera 2121a configured to image fluorescence in a surgical field, and at least one second camera 2122a configured to produce a non-fluorescence image of the surgical field. The processor 2130 can be configured to receive images from the plurality of cameras 2121a, 2122a, and to display on the display 2110 a fluorescence image from the at least one first camera 2121a and to display on the display 2110 the non-fluorescence image from the at least one second camera 2122a. As shown in FIG. 31, the processor 2130 can advantageously include a plurality of processors 2131a, 2132a, e.g., a separate processor for each camera within the plurality of cameras 2120. For example, at least one first processor 2131a can be configured to receive an image from at least one first camera 2121a and to display on the display 2110 a fluorescence image. In addition, at least one second processor 2132a can be configured to receive an image from at least one second camera 2122a and to display on the display 2110 the non-fluorescence image.

The display 2110 can be a primary display, a surgeon display, an assistant display, possibly other displays, or any combination of these. The display 2110 can include a display portion, a display, or display device as described herein. For example, in some embodiments, the display 2110 can include a display (or display portion) to be viewed through one or more oculars, e.g., a display within the viewing platform 9 of the surgical viewing system 1 shown in FIGS. 1, 3, 4A, 5A and 5B. The display (or display portion) could be within a housing. In other embodiments, the display 2110 can include a display mounted on a display arm from the ceiling or on a post, e.g., a display device 13 on display arm 5 of the surgical viewing system 1 shown in FIG. 1.

In various embodiments, the plurality of cameras 2120 can include a camera to provide a surgical microscope view of the surgical field. In some embodiments, the plurality of cameras 2120 can include a camera disposed on a surgical tool or on another medical device. The plurality of cameras 2120 can include at least one first camera 2121a and at least one second camera 2122a configured to form a left-eye view of the surgical field. The plurality of cameras 2120 can also include at least one first camera 2121b and at least one second camera 2122b configured to form a right-eye view of the surgical field. In some embodiments, the left and right-eye views are for stereoscopic viewing of the surgical field and the cameras can be angled to provide desired convergence mimicking the human eye. One or more cameras 2121a, 2121b, 2122a, and/or 2122b of the plurality of cameras 2120 can include optical assemblies as described herein. For example, one or more cameras 2121a, 2121b, 2122a, and/or 2122b can include a turning prism 54, a lens train 55, and/or a sensor 56 as shown in FIG. 6A.

As described herein, for the left-eye view, the at least one first camera 2121a can be configured to image fluorescence in a surgical field, and the at least one second camera 2122a can be configured to produce a non-fluorescence image of the surgical field. Similarly, for the right-eye view, the at least one first camera 2121b can be configured to image fluorescence in a surgical field, and the at least one second camera 2122b can be configured to produce a non-fluorescence image of the surgical field.

In some embodiments, the first camera 2121a and/or 2121b can be sensitive to infrared wavelengths, ultraviolet wavelengths, or other fluorescence wavelengths. For example, an optical detector, e.g., sensor 56 or an array of sensors, of the first camera 2121a and/or 2121b can be sensitive to fluorescence wavelengths. In some embodiments, the first camera 2121a and/or 2121b sensitive to fluorescence wavelengths can include an infrared, ultraviolet, or other fluorescence light source. In some embodiments, illumination using an optical fiber can be used to provide pump radiation to induce fluorescence. In some embodiments, a filter may be used to selectively direct fluorescence wavelengths to the first camera 2121a and/or 2121b sensitive to fluorescence wavelengths. In some embodiments, the second camera 2122a and/or 2122b may not be sensitive to fluorescence wavelengths.

In some embodiments, the processor 2130 can be configured to superimpose the fluorescence image over the non-fluorescence image. In other embodiments, the processor 2130 can be configured to superimpose the non-fluorescence image over the fluorescence image. In various embodiments, the processor 2130 can electronically process and synchronize the fluorescence and non-fluorescence images together. For example, the processor 2130 can read, align, and combine together the images.

The processor 2130 can include a general all-purpose computer and in some embodiments, a single processor may be used with both the left and right display portions 2110. However, various embodiments of the medical apparatus 2100 can include separate processing electronics for the left-eye and right-eye views. Such separate processing for the left and right channels can be advantageous over a processor with single processing electronics or the general all-purpose computer since time is critical in surgical procedures. For example, in some embodiments, having separate dedicated processing electronics for each channel can provide pure parallel processing, which results in faster processing of images, thereby reducing latency. In addition, addressing a failure of a general all-purpose computer may entail rebooting of the computer and involve some downtime. Furthermore, with separate processing electronics in left-eye and right-eye view channels, if one of the processing electronics were to fail, the processing electronics in the other channel can continue to provide images to the surgeon. Such redundancy can also be incorporated into a monocular viewing system. For example, in some embodiments of a monocular viewing system, two channels similar to a binocular viewing system can be provided. Images for the monocular viewing system can be split into each channel, with each channel having its own processing electronics.

Furthermore, in some even more advantageous embodiments, as shown in FIG. 31, the medical apparatus 2100 can include separate processing for each camera within each channel to further increase processing of images and reduce latency. For example, for the left-eye view, processor 2131a can be configured to receive an image from camera 2121a and to display on the display 2110 a fluorescence image from camera 2121a. Processor 2132a can be configured to receive an image from camera 2122a and to display on the display 2110 the non-fluorescence image from camera 2122a. The fluorescence and non-fluorescence images can be superimposed optically on the display 2110. Similarly, for the right-eye view, processor 2131b can be configured to receive images from camera 2121b and to display on the display 2110 a fluorescence image from camera 2121b. Processor 2132b can be configured to receive images from camera 2122b and to display on the display 2110 the non-fluorescence image from camera 2122b. The fluorescence and non-fluorescence images can be superimposed optically on the display 2110.

In certain embodiments, each of the separate processing electronics can be configured for image manipulation, e.g., to receive image data, process the image data, and output the images for display. For example, each of the processing electronics can be configured to receive one or more user inputs, receive one or more input signals corresponding to images from one or more cameras, and/or select which image to display. Each of the processing electronics can also resize, rotate, or reposition the selected image based at least in part on one or more user inputs. The processing electronics can also produce one or more output signals to drive one or more displays to produce one or more images. For example, each processing electronics can include a microprocessor, a field programmable gate array (FPGA), or an application specific integrated circuit (ASIC). Each processing electronics can also include a graphics processing unit (GPU) and random access memory (RAM). The processing electronics can also control the color balance, brightness, contrast, etc. of the one or more images.

In some embodiments, instead of superimposing fluorescence and non-fluorescence images, an image at a first wavelength range can be superimposed with an image at a second wavelength range. For example, one or more sensors can capture a first image at a first wavelength range, and one or more sensors can capture a second image at a second wavelength range. The first and second images can be superimposed optically as disclosed herein. As another example, the image at a first wavelength can be provided by narrow band imaging instead of fluorescence imaging. For example, a filter in some embodiments can allow imaging with the use of ambient light at blue (about 440 to about 460 nm) and/or green (about 540 to about 560 nm) wavelengths for the image at the first wavelength. Imaging at or near these wavelengths can improve visibility of features since the peak light absorption of hemoglobin occurs at these wavelengths. The image at the second wavelength can be provided without narrow band imaging (e.g., use of ambient light without a filter).

In further embodiments, the plurality of cameras 2120 can include different cameras for multiple views of the surgical site instead of or in addition to cameras mainly for imaging at different wavelengths. For example, in some embodiments, the plurality of cameras 2120 can include a camera providing a surgical microscope view, a camera disposed on a surgical tool, and a camera disposed on another medical device to provide different views of the surgical site. Some embodiments can also include a switch to determine which views are to be displayed either as superimposed, overlapping, adjacent, or as a monocular view. One or more image could also be from other sources, e.g., a data file, a computed tomography (CT) scan, a computer aided tomography (CAT) scan, magnetic resonance imaging (MRI), an x-ray, ultrasound imaging instrument, etc.

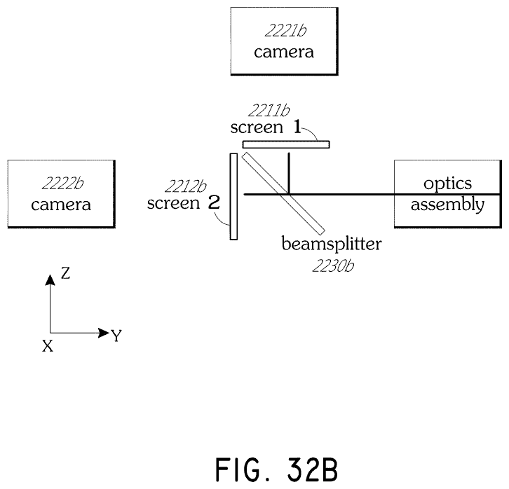

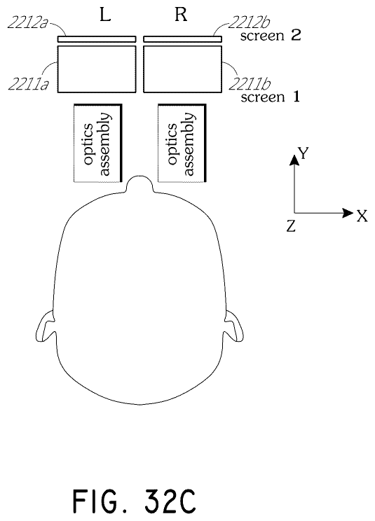

FIG. 32A schematically illustrates another example medical apparatus in accordance with certain embodiments described herein. Some such embodiments can also advantageously decrease the time to produce an image for viewing, which can be important in certain surgical procedures. For example, the medical apparatus 2200 can include a plurality of displays (or display portions), a plurality of cameras, and one or more beam combiners. As shown in FIG. 32A, to form a left-eye view, the plurality of cameras can include at least one first camera 2221a configured to produce a fluorescence image onto a first display 2211a and at least one second camera 2222a configured to produce a non-fluorescence image onto a second display 2212a. In some embodiments, the cameras 2221a, 2222a can produce the images onto the plurality of displays 2211a, 2212a, e.g., with a processor. However, in such embodiments, an electronic processor need not perform the combining of images. A beam combiner 2230a can be configured to receive the fluorescence and non-fluorescence images from the first 2211a and second 2212a displays and to combine or superimpose optically the fluorescence and non-fluorescence images for left-eye viewing, e.g., within a housing through an ocular or on a display device.

As shown in FIG. 32B, to form a right-eye view, the plurality of cameras can also include another first camera 2221b configured to produce a fluorescence image onto another first display 2211b and another second camera 2222b configured to produce a non-fluorescence image onto another second display 2212b. In some embodiments, the cameras 2221b, 2222b can obtain images that can be viewed on the plurality of displays 2211b, 2212b, for example, using processing electronics. However, in such embodiments, an electronic processor need not perform the combining of images. A beam combiner 2230b can be configured to receive the fluorescence and non-fluorescence images from the first 2211b and second 2212b displays and to superimpose the fluorescence and non-fluorescence images for right-eye viewing, e.g., within a housing through an ocular or on a display device.

In various embodiments, the beam combiner 2230 can include a beam splitter (e.g., a 45 degree or other angle splitter used in reverse), a dichroic beam splitter, a prism, or other optical structure to combine the beams. As an example, a beam combiner 2230a can be placed within the left-eye optical path to receive the fluorescence and non-fluorescence images from the first 2211a and second 2212a displays and to superimpose the fluorescence and non-fluorescence images for left-eye viewing, e.g., within a housing through an ocular or on a display device. Similarly, another beam combiner 2230b can be placed in the right-eye optical path to receive the fluorescence and non-fluorescence images from the first 2211b and second 2212b displays and to superimpose the fluorescence and non-fluorescence images for right-eye viewing. Some embodiments can further include imaging optics (e.g., an optics assembly) disposed to collect light from the displays to enable the images to overlap. The imaging optics can be configured to form images at infinity. FIG. 32C schematically illustrates a top view of an embodiment of a medical apparatus incorporating the example left and right assemblies from FIGS. 32A and 32B.

In some embodiments, instead of superimposing fluorescence and non-fluorescence images, an image at a first wavelength range can be superimposed with an image at a second wavelength range. For example, a first camera 2221a can produce a first image at a first wavelength range onto a first display 2211a, and a second camera 2222a can produce a second image at a second wavelength range onto a second display 2212a. The beam combiner 2230a can optically superimpose the first and second images. As another example, the image at a first wavelength can be provided by narrow band imaging instead of fluorescence imaging, and the image at the second wavelength can be provided without narrow band imaging as described herein.

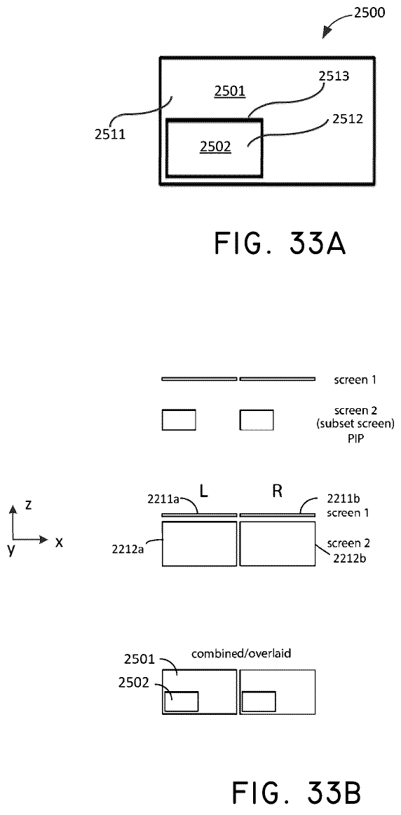

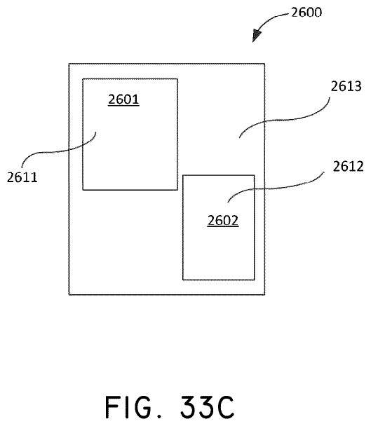

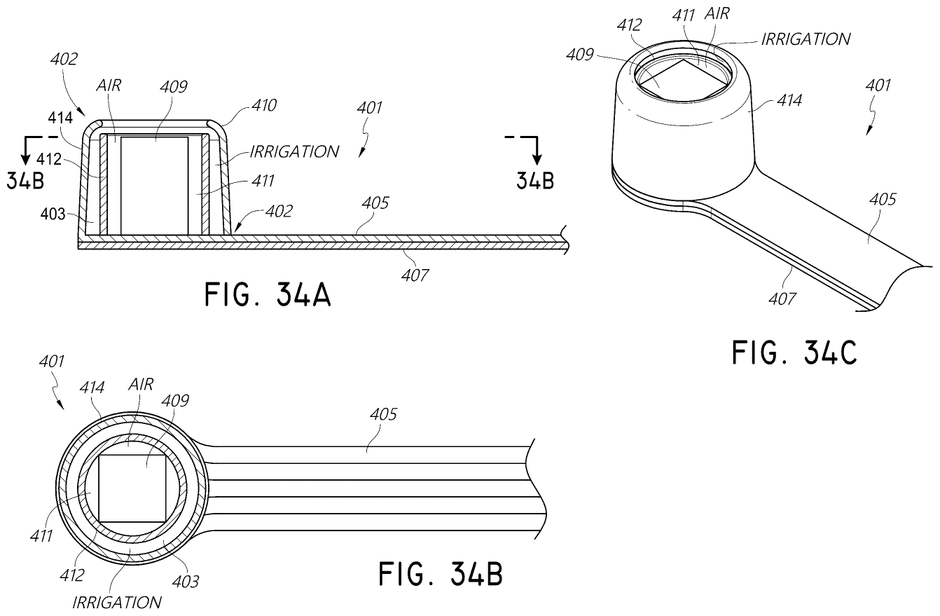

In addition, images from two different cameras of the same or substantially the same wavelength, but having other properties can be superimposed. For example, one image could be a natural image of tissue, and another view could be an unnatural image (e.g., an image with false color or an image with exaggerated or extreme contrast). In some embodiments, such superimposed images can advantageously show margins between healthy and unhealthy tissue. The example embodiments of the medical apparatuses shown in FIGS. 31 and 32A-32C can also be modified to produce a composite image of two or more images. FIG. 33A illustrates a schematic of an example composite image 2500, where a first (e.g., a background) image 2501 is produced on a first portion 2511 of the composite image 2500, and a second (e.g., a picture-in-picture (PIP)) image 2502 is produced on a second portion 2512 of the composite image 2500. In some embodiments, the images can include a fluorescence image and a non-fluorescence image. However, in other embodiments, the images are not necessarily fluorescence and non-fluorescence images. For example, one image can be a surgical microscope view of the surgical field from a camera producing the surgical microscope view. The other image can be the image of the surgical field from a camera disposed on a surgical tool or other medical device. One or more image could also be from sources other than cameras, e.g., a data file, a computed tomography (CT) scan, a computer aided tomography (CAT) scan, magnetic resonance imaging (MRI), an x-ray, ultrasound imaging instrument, etc. FIG. 33B schematically illustrates a front view of an embodiment of a medical apparatus incorporating the example left and right assemblies from FIG. 31 or 32A-32C to produce a composite image of two or more images for both left and right eyes.

Referring to the example embodiment shown in FIG. 31, for the left-eye view, the first camera 2121a can be a camera producing a surgical microscope view, and the second camera 2122a can be a camera disposed on a surgical tool or other medical device. Similarly, for the right-eye view, the first camera 2121b can be another camera producing a surgical microscope view, and the second camera 2122b can be another camera disposed on a surgical tool or other medical device. For each eye's view, the first camera 2121a, 2121b can produce the background image 2501 of the composite image 2500, and the second camera 2122a, 2122b can produce the PIP image 2502 in the composite image 2500. For the left-eye view, the processor 2131a can be configured to receive an image from the first camera 2121a and to display on the display 2110 the image as the background image 2501 of the composite image 2500. In addition, the processor 2132a can be configured to receive an image from the second camera 2122a and to display on the display 2110 the image as the PIP image 2502 of the composite image 2500. For the right-eye view, the processor 2131b can be configured to receive an image from the first camera 2121b and to display on the display 2110 the image as the background image 2501 of the composite image 2500. In addition, the processor 2132b can be configured to receive an image from the second camera 2122b and to display on the display 2110 the image as the PIP image 2502 of the composite image 2500. As shown in FIG. 33B, the position of the PIP image 2502 in the composite image 2500 can be in the same or different from that illustrated in the figures. Additional cameras or sources can also be used to produce a multiple PIP images.

Referring to the example embodiment shown in FIGS. 32A-32C, a beam combiner 2230a, 2230b can be placed within each eye's optical path to produce the composite image 2500. In some embodiments, the background image from a camera can be resized or the row count of pixels of the background image can be reduced. For example, the background image can be resized from the full frame to the size of the first portion 2511 (e.g., about ½, ⅔, ¾, etc.) of the composite image 2500. The beam combiner 2230a, 2230b in each eye's optical path between the viewer and the displays can superimpose the background image with a PIP image such that the background image appears on the first portion 2511 of the composite image 2500, and the PIP image forms within the remaining portion 2512 (e.g., about ½, ⅓, ¼, etc.) of the composite image 2500. In some embodiments, the remaining portion 2512 can include a border 2513 surrounding the PIP image 2502 to help prevent the viewer from seeing similar types of images as being falsely contiguous (e.g., similar types of tissues from multiple sources).

With reference to FIG. 32A, an example illustration using the left-eye view will be provided. The example illustration can also apply to the right-eye view in certain embodiments. For example, a first camera 2221a for providing a surgical microscope view can provide the background image on a first display 2211a, and a second camera 2222a disposed on a surgical tool or other medical device can provide the smaller image on a second display 2212a. The beam combiner 2230a can produce the background image from the first display 2211a as the first portion 2511 (e.g., about ⅔) of the composite image 2500. The beam combiner 2230a can also combine the PIP image from the second display 2212a as part of a second portion 2512 (e.g., about ⅓) of the composite image 2500. As shown in FIG. 33A, the background image 2501 can be produced in the majority (e.g., about ⅔) of the composite image 2500. The PIP image 2502 can be produced as part of, e.g., within the remaining portion 2512 (e.g., about ⅓) of the composite image 2500.

The display 2211a for the background image can be a 5″ display. The smaller PIP image from the second camera 2222a can be displayed on a smaller panel viewed off from the beam combiner 2230a, or could be displayed on a 5″ display using only a portion of the display (e.g., about ⅓ of the display or about part of ⅓ of the display). After properly baffling the optical pathways, the viewer can see the smaller image 2502 adjacent the background image 2501 as though it were a picture-in-picture.

The beam combiner 2230 can also produce additional PIP images from other displays as part of the composite image 2500. For example, multiple images (e.g., two, three, four, five, six, nine, twelve, etc.) from multiple displays (e.g., two, three, four, five, six, nine, twelve, etc.) can be viewed for each eye's view by using one or more beam combiners 2230.

In some embodiments, the smaller images can be superimposed with a dark (e.g., black) or light (e.g., clear) border to prevent the viewer from seeing similar images as being falsely contiguous (e.g., similar types of tissues from multiple sources). For example, after resizing the background image (e.g., to about ⅔ size), the remaining portion (e.g., about ⅓) of the image can be left black. The smaller images from other displays can be superimposed onto the black portion of the background image such that the images do not appear falsely contiguous. In addition, the border can help facilitate the beam combiner 2230 arrangement, making the alignment less critical in some embodiments. In some embodiments, the smaller images could be superimposed onto the background image. For example, the background image could include additional superimposed or overlapping images. Some embodiments can include a switch to determine which image to be displayed. For example, the background image could be switched off and not be displayed so that a different image(s) can be displayed in the first portion 2511 of the view 2500.

FIG. 33C illustrates that the images are not restricted to PIP images. FIG. 33C shows, for example, a schematic of an example view 2600 of multiple images (e.g., from multiple sources) of the surgical field disposed adjacent to one another. For example, a first (e.g., a background) image 2601 is produced on a first portion 2611 of the view 2600, and a second (e.g., a smaller or of similar size) image 2602 is produced on a second portion 2612 of the view 2600 such that the images do not necessarily overlap one another or do not need to substantially overlap, or one image does not need to be substantially contained within the other images. In such embodiments, the images can appear adjacent to one another or tiles in a manner that is not restricted to a PIP arrangement. As described above, more than two images may be included, for example, tiles with respect to each other. Additionally, more than one beam combiner and more than two displays may be employed in various embodiments to combine images, for example for the left eye (or for the right eye).

As described herein, some embodiments as shown in FIG. 32A-32C can, by the use of beam combiners 2230, advantageously can reduce latency by decreasing the time to produce an image for viewing. For example, multiple images can be tiled to view the multiple images from a variety of sources as opposed to being aligned and combined using an image processing technique that consumes computing power. In addition, an advantage of additional displays in each eye's path in certain embodiments can present to the viewer superimposed images without the complexity of electrical registration and timing issues. In some such embodiments, the brain can also merge the images if the additional displays are reasonably aligned optically.

### Camera Cleaning

Positioned within the body, the surface of the cameras or optical elements, such as lenses, can become fogged or otherwise obstructed (e.g., with blood). At least one of the cameras may be disposed on a surgical device. For example, at least one camera may be disposed on a retractor. In addition, at least one camera may be disposed on a surgical tool. In various surgeries, blood or other body fluids and/or biomaterial may be disposed on the camera and block or limit the field of view of the camera, or otherwise degrade the images produced by the camera. To maintain visual clarity, in some embodiments, the cameras can be cleansed while remaining in place within the surgical site. Thus, the cameras can be configured to be cleaned while on the surgical device, retractor, and/or surgical tool. In various embodiments one or more cameras outfitted with cleaning apparatus are included on retractors, tools, or both. A central hydraulic system can be used to provide hydraulic fluid (e.g., saline) and/or air to provide said cleaning for said retractor cameras, surgical tool cameras, or both. Accordingly, in various embodiments, surgical visualization systems comprise retractor cameras and surgical tool cameras each having cleaning apparatus for cleaning said cameras.

One approach to cleansing cameras is to provide pulses of fluid over the surface of the sensor or lens, thereby clearing any obstruction. Cleansing fluids may be, for example, distilled water, deionized water, or saline (including possibly physiological saline), among others. In some embodiments, these pulses may be brief, high-pressure, and low-volume. The pulse can be produced in a number of ways, for example, using a pop off valve (e.g., disposable elastomeric pop off valves) and a three way valve as discussed elsewhere herein. Pulses can also be produced other ways including by a diaphragm, actuated by a cam and motor in conjunction with a one-way valve. Fluid pressure can be supplied by air pressure in double spike IV bottle. A disposable diaphragm pump can be used to increase pulse pressure. In some embodiments, two pumps can be used to eliminate interruption in pulse pressure. In some embodiments, passive hydraulic amplifiers can be used to increase the fluid pressure. In some embodiments, solenoids, piezoelectric actuators, or other techniques may be used. A rolling edge diaphragm, Bourdon tube, or bellow can likewise be used to produce the pulse. In one embodiment, a reed valve can be configured to alternate between air and saline operating at the natural mechanical resonance frequency of the reed valve and associated fluid and air column dynamics. Pressure may then be maintained in the air and fluid circuits just below valve opening pressure, and an electrical signal may increase pressure until the reed valve opened, thus avoiding pulsating fluid tubing. In various embodiments, the fluid can be saline or other biocompatible liquid. In some embodiments, the lens elements are configured such that a stop is affixed to a first lens element wherein the stop covers a large fraction of the first lens element. Additionally, in some embodiments, the lens elements are configured such that a first element comprises a plano window. The stop can be located behind the plano window or more lens elements and can be relatively small. In such embodiments, the light collected by the stop is correspondingly small such that the area of the plano window that should remain clean is relatively small. This configuration can facilitate cleaning the lens system. In some embodiments, the plano window is secured to the lens system using a structure that does not extend over the top of the plano window so as to not interfere with mechanisms configured to clean the lens system. For example, the plano window can have a step edge or be retained by a support member (e.g., a metal ring or an edge of the lens system housing) that extends along a side portion of the plano window without extending beyond the distal face of the plano window through which light is collected for the sensor.

In some embodiments, a high-pressure fluid pulse can be followed by a high-pressure pulse of air or gas in order to squeegee or dry the surface of the camera or lens and prevent salt deposits or image obscuration. In some embodiments, the pressure of the fluid pulse and/or air or gas pulse can range from about 20 psi to 60 psi. For example, the pressure of the fluid pulse and/or air or gas pulse can be about 20 psi, 30 psi, 40 psi, 50 psi, 60 psi, or a pressure in between any of these values. In some embodiments, the pressure of the fluid pulse and/or air or gas pulse can be about 60 psi. Higher or lower pressures are also possible. The pressure of the liquid pulse and/or air or gas pulse may for example be as high as 70, 80, 90, 100 psi or higher or as low as 10 psi or lower. In various embodiments, the pressure ranges from about 50 psi to about 70 psi, for example, about 60 psi. The air or liquid may in some embodiments be ejected in a diverging manner although the air or liquid could be ejected in a more narrow focus straight spray. In various embodiments, the sources for the high-pressure air may be, for example, hospital compressed air systems, or compressed air tanks or possibly air compressors. The air pulse can be actuated similar to the fluid pulse as described above. For example, pop off valves (e.g., disposable elastomeric pop off valves) and a three way valve as discussed elsewhere herein can be used. In some embodiments, a diaphragm actuated by a cam and motor in conjunction with a one way valve may be used. In some embodiments, a Venturi effect may be used to generate the post-wash air flow. The Venturi effect depends on the size and shape of the port through which the fluid flows. In general, when a fluid flows through a constricted section of pipe, the pressure is reduced. This low pressure can draw in additional outside air and can cause air flow following the fluid pulse. As discussed in more detail below, a proportional foot pedal can control actuation of the fluid and/or air pulses.

In some embodiments, the camera cleaning sequence may comprise alternate delivery of liquid and air/gas multiple times. As an illustrative example, actuation of the camera cleaning system may cause the system to deliver pulses of liquid and air/gas three times. Thus, the camera cleaning system may deliver a pulse of liquid followed by a pulse of air/gas a first time, then a pulse of liquid followed by a pulse of air/gas a second time, then a pulse of liquid followed by a pulse of air/gas a third time. The number of pulses can be larger or smaller. Additionally, the sequence of liquid and air/gas pulses can vary. In other embodiments, for example, the camera cleaning sequence may comprise the delivery of a pulse of liquid, followed by the delivery of a pulse of air/gas, without further delivery of liquid or air/gas, until the camera cleaning system is actuated again. Additionally, multiple pulses of liquid can be grouped together as can multiple pulses of air or gas. In one illustrative example, for instance, two pulses of liquid can be followed by two pulses of air or gas. Or a plurality of pulses of liquid can be followed by a pulse of air or gas. Multiple pulses of air or gas may also follow a pulse of liquid. A wide range of sequences are possible and as discussed below may be selected by the user, for example, via a graphic user interface or other interface. Custom sequences may be selected or programmed by the user or pre-programmed for selection by the user or may be programmed in the system without possible modification by the user.

In some embodiments, the duration of the pulses may be 100-200 milliseconds. The pulse duration, however, may be longer or shorter. The pulse duration of the liquid can be the same as the pulse duration of the air/gas or alternatively the pulse duration of the liquid may be different (e.g., longer or shorter) than the pulse duration of the air or gas.

As referred to above, multiple parameters of the camera cleaning system may be programmed by the user, e.g., the surgeon, assistant, nurse or technician. For example, the duration of the pulses of liquid and/or air/gas may be programed. In addition, in some embodiments, the time that elapses between each pulse of liquid and air/gas may be programmed although in various embodiments, for example, where a three way valve is employed, the transition from fluid pulse to air/gas pulse will be virtually instantaneous. In embodiments where the camera cleansing system comprises alternate delivery of liquid and air/gas multiple times, the time that elapses between one round of delivering a pulse of liquid followed by a pulse of air/gas, before delivering another round of a pulse of liquid followed by a pulse of air/gas, may be also be varied and customized by the user by programming using an interface or may be pre-programed options or possibly no option is provided via pre-programing.

Accordingly, in some embodiments, the camera cleansing system may be pre-programmed by the manufacturer. In addition, the camera cleansing system may be programmed by the surgeon or other operator. In some embodiments, the surgeon or other operator may decide to alter the parameters of the camera cleansing system pre-programmed by the manufacturer. For example, depending on the surgical procedure, the surgeon or other operator may desire a different duration of the fluid and air/gas pulses. As another example, depending on the surgical procedure, the surgeon or other operator may desire a different number of pulses to be ejected with an actuation of a foot pedal or other form of input device. As another example, the surgeon or other operator may desire a different pressure for the pulses and/or volume of liquid or air to be ejected.

Actuation of the camera cleansing system may be automatic in some embodiments. For example, the camera cleansing system may be programmed to clean the cameras automatically every few minutes. In some embodiments, as discussed below, pulses can be automatically initiated upon detection of a feature of the image obtained from the camera, for example, if the color of the image is predominantly the color of blood, indicating blood is on the camera, or the spatial resolution degrades, etc.

In addition, actuation of the camera cleansing system can be manually controlled. For example, a proportional foot pedal can control actuation of the liquid and/or air/gas pulses. For example, depression of the foot pedal can actuate the delivery of liquid and/or air/gas pulses to the camera(s). In some embodiments, the foot pedal may be configured to actuate the delivery of a single liquid and air pulse combination when initially depressed and depressing the foot pedal further to the ground may increase the number of and/or rate of liquid and air/gas pulses delivered to the camera(s). Two or more distinct regions of foot pedal depression may thus be provided that cause pulses with different parameters to be delivered so as to give the surgeon more control of the pulses delivered. In some embodiments, the foot pedal is not proportional. In this manner, possibly the number of liquid pulses, the number of liquid and air pulse combinations, the pulse pressure, or other parameters can be controlled. In some embodiments, the fluid and/or air/gas pulses can be voice controlled. In some embodiments, the fluid and/or air/gas pulses can be controlled via a touchscreen. In some embodiments, the graphical user interface or other control can enable control of fluid and/or air/gas delivery to the cameras for cleaning the cameras and pulse washing and/or air drying cameras. Other input devices can also be used.

In various embodiments, the liquid, air, or gas delivered to the camera(s) may be provided by a plurality of fluid lines. The fluid lines may include an inlet at one end connected to a fluid source and an outlet at the other end. In some embodiments, the outlet includes a nozzle or is in fluid communication with a nozzle configured to deliver fluid to the camera(s) to be cleaned.

In various embodiments, these lines may connect to a hydraulic and/or pneumatic cassette used in a hydraulic and/or pneumatic system with pressurized hydraulic and/or pneumatic supplies. In various embodiments, the cassette comprises elastomeric proportional valves. In some embodiments, valves in the cassette control flow of the liquid and air to deliver the pulses to the cameras. As discussed elsewhere herein, in certain embodiments, pop off valves (such as disposable elastomeric pop off valves) may be utilized to control the delivery of liquid and/or air/gas to the camera(s). In some embodiments, three way valves connected to the liquid (e.g. saline source) and to the air source may be employed to switch from a liquid pulse to an air pulse while reducing dribble. Other configurations, however, may be employed.

In some embodiments, pinch valves for controlling the flow emission of pulses can be connected to the liquid and gas lines that extend from the cassette to the retractor. Actuating the pinch valves can cut off the supply of liquid or air/gas to the camera(s), while releasing the pinch valve may open the supply of liquid or air/gas. In some embodiments, actuation of the pinch valves can be driven by solenoids. In some embodiments, the pinch valves may be disposable roller pinch valves or thumb wheel valves.

In embodiments comprising a plurality of cameras, liquid and/or air/gas pulses can be delivered to each of the plurality of cameras simultaneously. In these embodiments, the cameras can be connected to the same fluid line in communication with the pressurized fluid source. In addition, one valve (e.g., pop off valve, pinch valve, roller or thumb wheel valve etc.) can be connected to the fluid line connecting the high pressure fluid source to the cameras. In some embodiments, this fluid line splits into or is coupled to multiple lines directed to the different cameras or groups of cameras. Thus, opening the one valve may cause all the cameras to be cleaned at the same time. Similarly, closing the one valve may cease the delivery of liquid and/or air/gas to all the cameras at the same time. Advantageously, including only one valve with the camera cleansing system can potentially reduce complexity, cost, and bulk to the camera cleansing system, compared to adding multiple valves to the camera cleansing system.

In other instances, liquid and/or air/gas pulses can be delivered to each of the plurality of cameras separately and individually. In these embodiments, each camera or groups of cameras may be connected to separate individual liquid and/or air/gas lines. In addition, multiple valves (e.g., pop off valve, pinch valve, roller or thumb wheel valve, etc.) can be connected to these separate fluid lines. For example, one valve can be connected to each fluid line dedicated to a respective camera or stereo camera pair. For example, one pop off valve can be connected to each fluid line dedicated to a respective camera or stereo camera pair. Advantageously, utilizing multiple valves can allow the liquid and/or air/gas delivery to be controlled to each camera separately and individually. Thus, for example, if one camera is dirty, only that camera may receive pulses of liquid and air/gas, thus allowing the views of the other clean cameras to avoid obscuration by pulses of liquid or air/gas.

Other variations are possible. For example, one valve can be connected to a fluid line dedicated to a respective retractor blade to control delivery of liquid and/or air/gas. Each retractor blade may comprise two or more cameras. Thus, for example, one valve may control the delivery of liquid and/or air/gas to a pair of cameras on one retractor blade. This valve may control the washing of the cameras on that retractor blade independent of the washing of one or more cameras on a separate blade. In some embodiments, the camera cleansing system may be configured so that the front lens or window of each channel (left and right channels) of stereo cameras is cleaned at the same time. This same approach applies to other types of retractors such as tube retractors. Groups of cameras on such retractors may, for example, each be controlled by separate valves. Multiple groups of cameras may therefore be independently controlled.

In some embodiments, the valves can come packaged with disposable tubing. The disposable tubing can comprise an input configured to connect to the hydraulic supply cassette and an output configured to deliver fluid to a camera. In some embodiments, the packs can support multiple cameras, such as 2, 4, or 6 cameras. In some embodiments, the packs can include multiple individual tubes, one for each camera or camera pair. In other embodiments, the packs can include a tube that splits into multiple outlets corresponding to the number of cameras configured to be supported by the pack. For example, a pack that supports 2 cameras may include a tube that splits into two outlets at one end of the tube. That same tube may include one input at the other end of the tube.

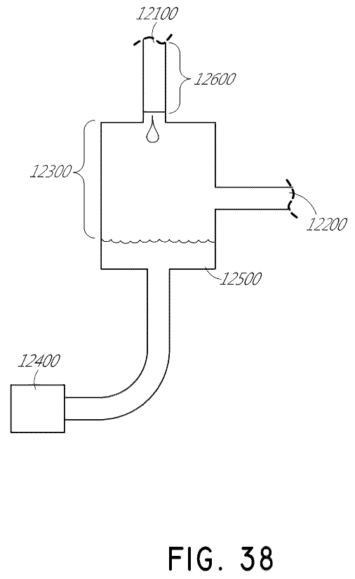

In various designs, electrical isolation is provided to reduce the risk of electrical shorts. Referring to FIG. 38, there is illustrated one embodiment of a hydraulic line with an inlet 12100 connected to a fluid source, a vertical drip member 12600, an outlet that comprises a nozzle 12400, a reservoir 12500 partially filled with fluid, an air gap 12300 in the reservoir 12500, and pressurized air 12200 that pushes the liquid in the reservoir 12500 through and out the nozzle 12400. The air gap 12300 can provide electrical isolation. Thus, the air gap 12300 can be utilized to electrically decouple the liquid delivered to the camera(s) from the electronics of the system and help ensure safety and reduce risk of damage to the equipment.

In some embodiments, the flex cable may include fluidic channels to convey the air, gas, or liquid to the camera optics to provide for cleaning. Fluidic channels can also transport other fluids such as pharmaceuticals, saline for irrigation, fluorescent dyes, etc. to the surgical site. Fluidic channels can also be provided for aspiration, to provide egress of gases or liquids from the surgical site. The fluidic channel containing flex cable may be an overlay or surrounding member affixed over the electronic flex cable, thereby allowing the fluid-carrying component to be disposable, whereas the electronic flex cable with integrated optics module may be sterilizable and reusable. The distal end of the fluidic flex cable can contain an outer housing that is secured over the imaging module. In some embodiments, it is the annular space and shape of the inner surface of said outer housing that directs the fluid and or fluid air pulses over the most distal surface of the optics for cleaning.

FIGS. 34A-C show an embodiment wherein an irrigation pathway 403 is provided by an outer sheath 401 comprising a cable 405 including fluidic channel containing cable and a portion that covers the sensor and imaging optics 409. The portion 402 of the sheath 401 that covers the sensor and imaging optics 409 can be shaped to provide conformal fitting yet leave a space 411 between the sheath 401 and the sensor and imaging optics 409 for air flow. A section 410 of the outer sheath 401 forward of the imaging optics can be shaped to direct the fluid across the distal surface of the lens. In some embodiments, the outer sheath 401 that delivers the fluid can be a separable assembly that can be added to or attached to the optical stack 409. In some embodiments, the fluid is delivered in the cable 405 that forms part of this sheath 401. This cable 405 is separate from the flex cable 407 that includes electrical connection for powering and receiving signal from the camera. In some embodiments, when the separable assembly is attached to the optical stack 409 and electric flex cable 407, the fluidic cable 405 assembly sits on top of the electrical flex cable 407 and above the optical stack 409. In some embodiments, the outer sheath 401 can be designed to snap onto the optical portion creating a seal around the optical stack 409 and then later detached. In various embodiments, the detachable sheath 401 is disposable while the imaging optics 409, sensor, and flex wire 407 connected thereto are sterilizable. In other embodiments, a fluid nozzle can be positioned on one side of the imaging optics, with an air nozzle on the other side with no sheath.

In some embodiments, the outer sheath 401 at the location of the camera and imaging optics 409 includes an inner wall 412 in addition to an outer wall 414, both shaped as concentric right circular cylinders, one inside the other. In some embodiments, the inner wall 412 which is closest to the imaging optics 409 surrounds the optical stack so as to leave a gap 411 of air between the optical stack 409 and the cylindrical inner wall 412. This gap 411 advantageously facilitates air flow between the optical elements (e.g., lenses) in the optical stack and thereby reduces the risk of condensation forming on the optical elements. In various embodiments, the outer sheath 401 is configured so as to allow fluid to enter the outer sheath 401 and be directed over the front most surface of the imaging optics 409. As described above, the delivered fluid can be liquid, or gas, or a combination of both. In some embodiments, the irrigation pathway 403 can also be used to deliver other fluids such as pharmaceutical and fluorescent dies. In some embodiments, the irrigation pathway 403 can be used for aspiration and fluid egress.

Other configurations are possible. For example, in some embodiments, the fluid can be delivered by separate fluidic channels of the same cable that includes the electrical power and signal lines.

In some embodiments, liquid pulses are produced when blood or other obstruction is detected. The intensity level of the camera can be monitored to determine when visibility is compromised. In various cases, the color of the light reaching the camera can be analyzed to determine, for example, that blood is obstructing or impairing vision of the cameras and to thereby trigger pulse washing. The processing electronics could be utilized to analyze the image signal and determine whether pulse washing is to be initiated. In some embodiments, attenuation of the green wavelength in comparison to other wavelengths such as red, or absorption of the green wavelength, may indicate that blood is on the camera and reducing the amount of light entering the camera. In some embodiments, a modulation transfer function can be utilized as an indicator of whether the front lens or window of the camera is dirty. For example, a modulation transfer function may show attenuation possibly of the lower frequencies, which may indicate that the image is degraded as a result of material on the surface of the front window or lens of the camera. This foreign matter may a least partially block, scatter, or otherwise redirect and/or attenuate the light collected by the camera. Other types of image processing measurements and tests may be used to ascertain whether the image is degraded or altered by material on the front of the camera.

Example Camera Integrated with Cleaning

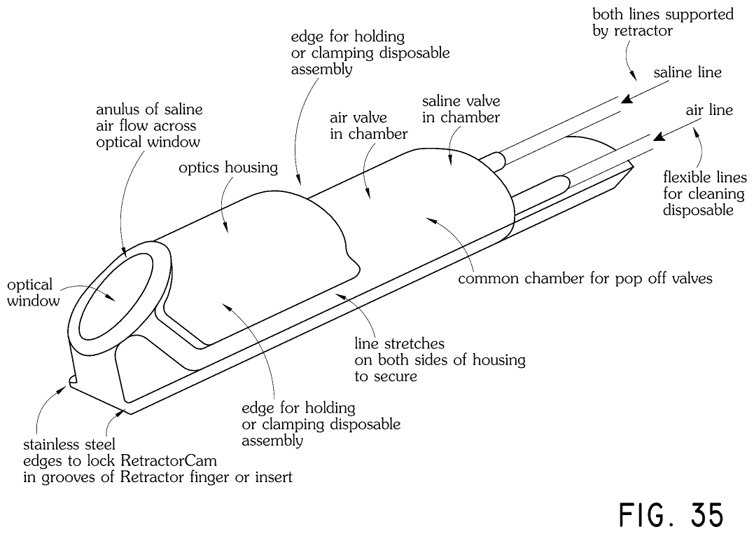

FIG. 35 shows a camera supported on a platform configured to lock into the blades or fingers of a retractor or into an insert such as for a tubular retractor as described above. In particular, metal, e.g., stainless steel, edges are shown for interfacing with the blades or fingers of the retractor or in the insert of the tubular retractor. These edges may fit into groove in the blades or of the tubular insert for the tubular retractor. The window of the camera optics is shown surrounded by an annulus through which air and saline can be ejected to clean the window. A saline line and an air line are shown for supplying pressurized saline and air. In some embodiments, pop off valve is included for the saline line and a pop off valve is provided for the air line to provide a pressurized pulse when the pressure exceeds a threshold. The pressurized pulse of saline exits the annular output port. In some embodiment, one or more (e.g., a pair or multiple pairs) of nozzles provide egress of liquid and gas for cleaning. This pulse of pressurized saline is followed by a pulse of pressurized air to force (“squeegee”) residual saline from the optical window. The shape and number of the exit ports for the saline and air proximal to the window may vary.

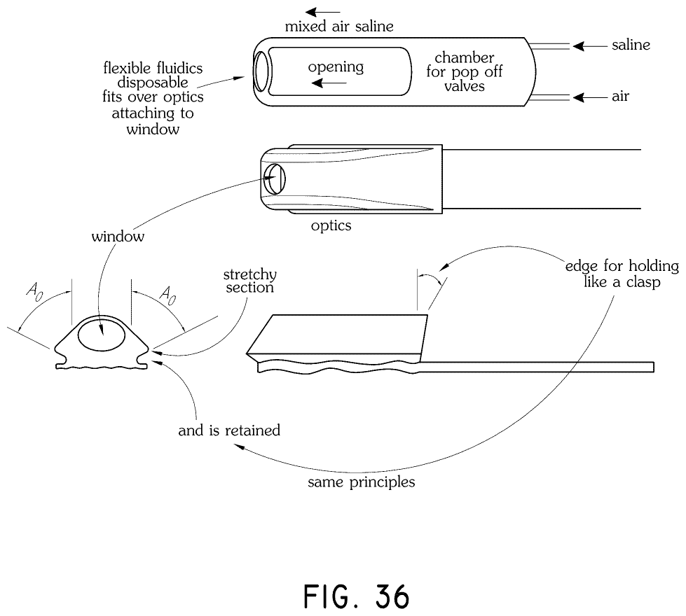

FIGS. 35 and 36 further illustrate a housing or a bladder for the camera fluidics used for cleaning the camera. The housing may be flexible and form fitted to the platform. In some embodiments, the housing is made of elastic material. One end of the housing may include a hole aligned with the optical window of the camera and the other end of the housing may include an edge for securing the housing to the platform in a manner similar to a hasp. In some embodiments, the housing can be slipped on over the optics, possibly by stretching the housing. In addition, the elastic material can assist in affixing the housing. Further, the housing may be configured to connect to the saline line and the air line when the housing is secured to the platform. In some embodiments, the housing may be disposable.

### Valves

Various embodiments described herein utilized valves. A number of different valves types can be employed.

In various embodiments, a valve comprises a linear actuator such as a linear motor, a member attached to the motor such that the motor can move the member, tubing having a pathway therethrough. The movable member is configured such that the motor can cause the movable member to contact the tubing to compress and close the pathway through the tubing. The motor can also cause the movable member to return from such a position so as to reduce the compression and open the pathway through the tubing. Moreover, the motor can cause the movable member to move back and forth in a linear direction between these positions thereby opening and closing the pathway at a rapid rate.

The resultant valve may be operated with the linear motor oscillating and thereby repetitively switching the pathway from an open to a closed state and back. The overall valve setting may be established by the duty cycle of the oscillation provided by the motor. The motor oscillation, for example, can be characterized as an oscillating waveform having a pulse width and/or duty cycle that can vary. By increasing the portion of time that the movable member compresses the tubing compared to the portion of time that the movable member is withdrawn and compresses the tubing less, the valve can be changed into a more closed state. In comparison, by decreasing the portion of time that the member compresses the tubing compared to the portion of time that the member is withdrawn more, the valve will be changed into a more open state. The waveform driving the motor can thus be modulated, for example, using pulse width modulation or frequency modulation (e.g., when the duty cycle is not 50%: 50%). This waveform can thereby determine how much time the moveable member spends compressing the tubing and thus the amount of resistance to the flow of fluid through the tube. Different types of motors and tubing and different configurations may be employed. In some embodiments the tubing may be disposable tubing.

Another type of valve comprises a pneumatically driven proportionally controlled fluid valve. This pneumatic valve can employ pressure (or vacuum) on opposite sides of a movable piston. The movable piston provides an occlusion to a fluid pathway. The movable piston itself may be the occlusion or may be physically attached to and drive a movable element that can be moved into or further into the fluid pathway to at least partially block the pathway as well as in a direction away from the pathway to reduce the amount that the moveable element blocks the pathway.

First and second pressures can be applied to respective opposite first and second sides of the piston. If the first pressure exceeds the second pressure the moveable element may be moved so as to increase blockage of the pathway. Conversely, if the second pressure exceeds the first pressure, the moveable element may be moved in the opposite direction so as to reduce the amount of blockage of the pathway. The first and second pressure, therefore, can be controlled to control the occlusion and thus the flow of fluid through the pathway.

The first and second pressures can be provided by air or gas on the respective first and second sides of the piston. (Alternatively, the first and second pressures can be provided by vacuum.) In this manner, a pneumatic proportional valve is provided. The opposing pressure provides for increased stability and control of the valve. In some embodiments, the first and second pressures on the respective first and second sides of the piston can be measured to provide feedback for adjusting the first and second pressures as may be desirable. Such feedback can further enhance the stability and control of the valve.

Other types of feedback besides pressure can also be used. For example, instead of pressure measurements, position measurement of the piston and/or occlusion can be employed. Hall effect encoding of the piston and/or moveable element can be used to track the position of the valve for feedback.

Accordingly, such a valve may include a housing for the piston and for providing first and second chambers on opposite sides of the piston. These chambers can be used to receive air or gas (or to be evacuated) to establish the first and second pressures respectively. Lines from pressurized air or gas sources (or vacuum pumps) can be in fluid communication with these chambers. Valves may be employed to control the flow of this air or gas into the first and second chambers. Processing electronic may also open and close the valves that flow air and gas into the first and second chambers. This processing electronics may receive feedback from one or more sensors such as the Hall effect sensors discussed above.

A valve similar in design but that uses hydraulic fluid rather than air or gas can be potentially be employed in other embodiments.

### Fluidic Pop Off Valves in Chamber

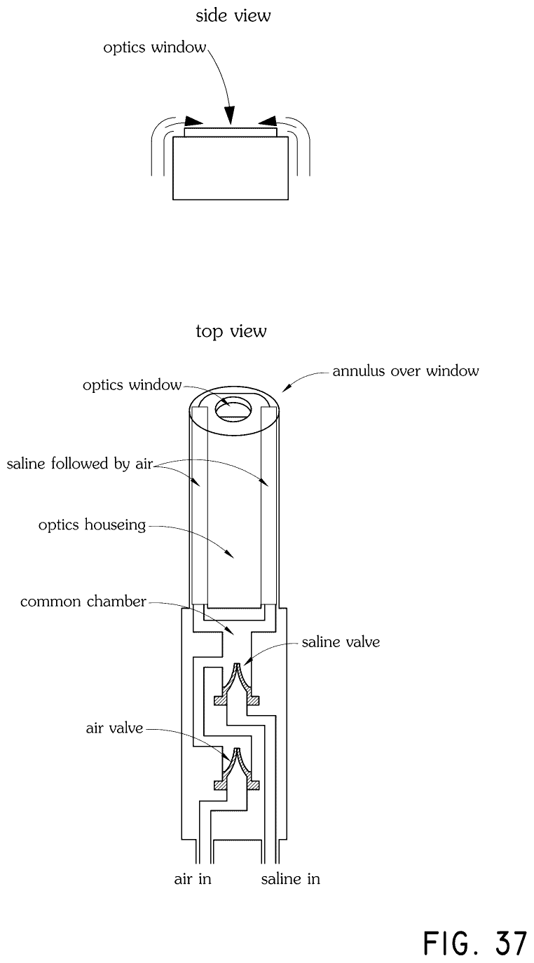

FIG. 37 represents a side cross sectional schematic view of one embodiment of fluidic pop off valves. These pop off valves may be encased in a housing or bladder that may be configured to be slipped onto a platform as described above with respect to FIGS. 35 and 36. Referring to FIG. 37, there is illustrated an air line comprising an air ingress, an air pop off valve (e.g., an elastomeric pop off valve), and a common chamber. Also illustrated is a saline line comprising a saline ingress, a saline pop off valve (e.g., an elastomeric pop off valve), and the common chamber. As illustrated, the saline valve may be disposed distal to the air valve. In addition, the common chamber, which is configured to receive both saline and air, may be disposed distal to the saline valve. With continued reference to FIG. 37, the common chamber may then extend to the camera optics in the optics housing, so that air and/or saline from the common chamber may travel to the camera optics in the optics housing and clean the camera optics.

In some embodiments, saline may be introduced into the saline ingress, and after the saline supply is shut off, air may be introduced into the air ingress. In operation, when saline is introduced into the saline line, pressure will build on the saline pop off valve until a pre-determined threshold is reached, at which point the saline pop off valve will open. Thereafter, saline may be travel through the common chamber and to the camera optics for cleansing the camera. When the saline supply is lowered or shut off, the pressure on the saline valve may drop, thus closing the saline pop off valve. As illustrated in FIG. 37, the saline pop off valve may be a one way valve. Thus, after saline cleanses the camera optics, and when the saline valve is closed, residual saline may remain in the common chamber, as the one way pop off saline valve may prevent saline from flowing back in a proximal direction out the saline ingress.

After saline is introduced into the saline ingress, air may be introduced into the air ingress. When the pressure of the air against the air pop off valve reaches a predetermined threshold, the air pop off valve may open, thus allowing the air to travel in a distal direction through the air line, into the common chamber, and to the camera optics for further removal of saline on the camera optics. Additionally, when the air travels through the common chamber, it may dry or blow out any residual saline in the common chamber as well as in the common lines/channels to the camera. Thus, running air and saline through a common chamber and common lines/channels to the camera may help prevent the build-up of residual saline in the common chamber and lines/channels to the camera and reduce the incidence of dribble.

### Kerrison

In various embodiments, the console can be equipped with a hydraulic and/or pneumatic system that can be employed to drive hydraulic and pneumatic tools.

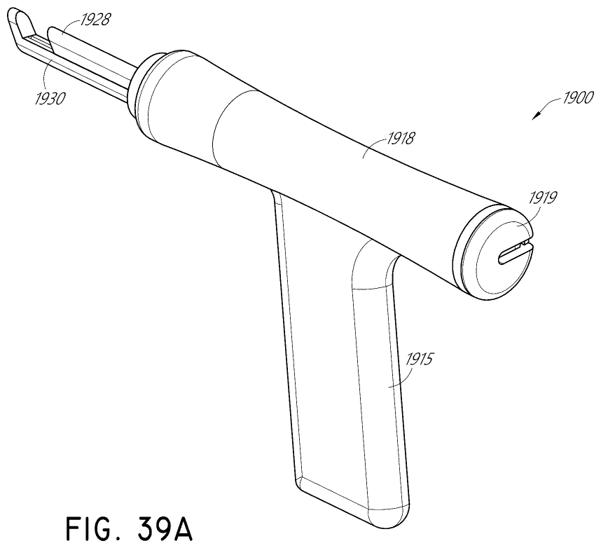

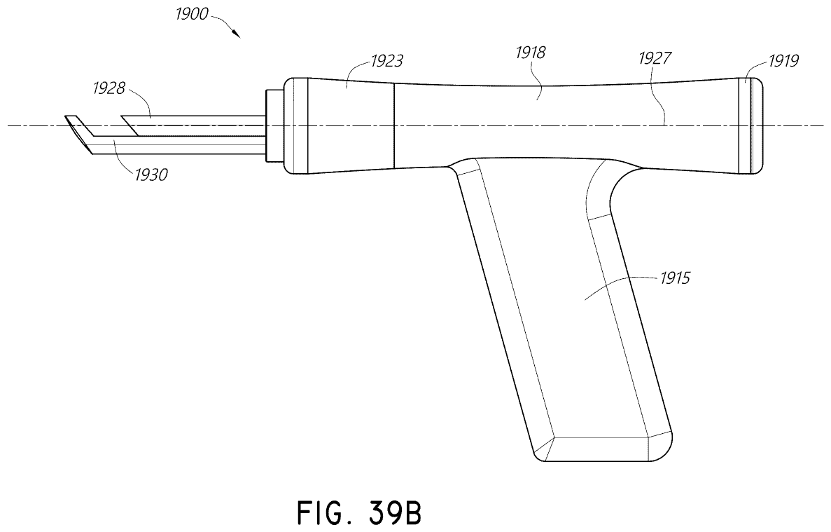

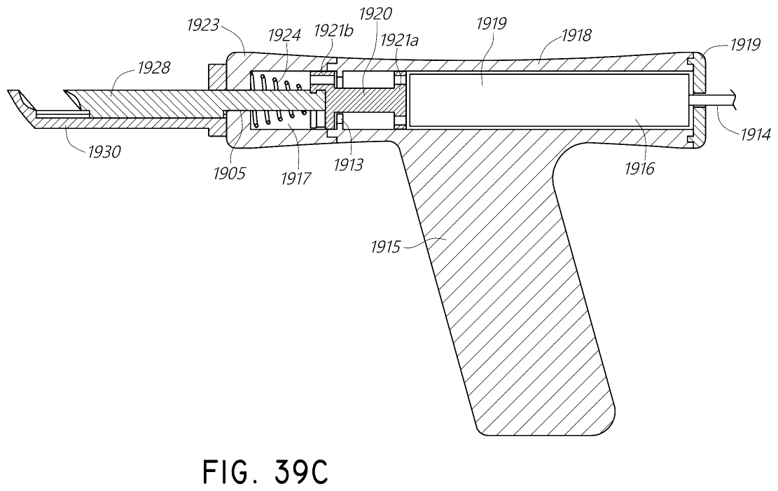

FIGS. 39A-C illustrate an embodiment of a Kerrison 1900 that can be operated hydraulically and/or pneumatically. The Kerrison 1900 can include a proximal handle portion 1918. The proximal handle portion 1918 can be attached or otherwise connected with a distal handle portion 1923. In some embodiments, the proximal handle portion 1918 includes a grip 1915 (e.g., a pistol grip or other ergonomic grip). The proximal handle portion 1918 can be configured to rotate about a handle axis 1927 (shown, e.g., in FIG. 39B) with respect to the distal handle portion 1923.

In some embodiments, the Kerrison 1900 includes a base 1930. The base 1930 can include a cutting portion at a distal end (e.g., the left end of FIG. 39B). The base 1930 can be fixed axially (e.g., parallel to the handle axis 1927) with respect to the distal handle portion 1923 and/or with respect to the proximal handle portion 1918. In some embodiments, the base 1930 and/or distal handle portion 1923 are rotatable about the handle axis 1927 with respect to the proximal handle portion 1918.

As shown in FIG. 39C, the proximal handle portion 1918 can define an actuation chamber 1919. In some embodiments, at least a portion of the actuation chamber 1919 along a length of the actuation chamber 1919 parallel to the handle axis 1927 has a substantially constant cross-section. In some embodiments, at least a portion of the actuation chamber 1919 has a circular cross-section.

In some embodiments, the distal handle portion 1923 defines a distal actuation chamber 1917. In some embodiments, the distal actuation chamber 1917 has a cross-section with substantially the same shape and/or size as a cross-section of at least a portion of the actuation chamber 1919.

The Kerrison 1900 can include a piston 1920. The piston 1920 can be operably coupled with and/or attached to a Kerrison top portion 1928. For example, the piston 1920 can be a unitary part with or attached/adhered/welded to the Kerrison top portion 1928. In some embodiments, the piston 1920 and top portion 1928 are connected via a releasable connection (e.g., a protrusion-slot connection). The piston 1920 can be fixed axially (e.g., parallel to the handle axis 1927) with respect to the top portion 1928. In some embodiments, the piston 1920 is fixed rotationally with respect to the top portion 1928 (e.g., rotation about the handle axis 1927).

The top portion 1928 can include a cutting edge on the distal end of the top portion 1928. The cutting edge of the top portion 1928 can be configured to operate with the cutting portion of the base 1930 to cut medical material (e.g., bone and/or other tissue). In some embodiments, the top portion 1928 is connected to the base 1930 via a track-protrusion engagement. For example, the top portion 1928 can include a protrusion configured to slidably engage with a track in the base 1930. Engagement between the track of the base 1930 and the protrusion of the top portion 1928 can limit the movement of the top portion 1928 with respect to the base 1930 to the axial direction (e.g., parallel to the handle axis 1927).

In some embodiments, the piston 1920 is configured to fit within the actuation chamber 1919 and/or within the distal actuation chamber 1917. For example, the piston 1920 can have a first guide portion 1921a configured to fit snugly within the actuation chamber 1919 (e.g., fit such that movement of the first guide portion 1921a within the actuation chamber is substantially limited to axial movement and rotational movement about the handle axis 1927). In some embodiments, the piston 1920 includes a second guide portion 1921b. The second guide portion 1921b can be configured to fit snugly within the distal actuation chamber 1917. Axial movement of the piston 1920 can be limited by interaction between a radially-inward projection 1913 of the proximal handle portion 1918. For example, proximal axial movement of the piston 1920 can be limited by interaction between the second guide portion 1921b and the radially-inward projection 1913. In some embodiments, distal axial movement of the piston 1920 is limited by interaction between the first guide portion 1921a and the radially-inward projection 1913.

The distal handle portion 1923 can include a distal opening 1905. The distal opening 1905 can be sized and/or shaped to accommodate passage of the top portion 1928 therethrough. In some embodiments, the top portion 1928 is sized and shaped to fit snugly within the distal opening 1905. For example, the top portion 1928 can have a non-circular cross-section sized to substantially match a cross-section shape of the distal opening 1905. In some embodiments, the top portion 1928 is rotationally locked to the distal handle portion 1923 via interaction between the distal opening 1905 and the top portion 1928. In some such embodiments, the grip 1915 can be rotated relative to the top portion 1928 and the base 1930. In some embodiments, sensors and/or optical devices (e.g., cameras, CMOS sensors, etc.) can be attached to the proximal handle portion 1918 such that the relative alignment of the sensors and/or optical devices with respect to the handle portion 1918 remains consistent independent of rotation of the top portion 1928 and base 1930 with respect to the handle portion 1918.

As illustrated in FIG. 39C, an actuation element 1916 can be positioned within the actuation chamber 1919. In some embodiments, the actuation element 1916 is an inflatable and/or disposable bag or balloon configured to be inflated/deflated with physiological saline and/or gas. In some embodiments, the Kerrison 1900 is a breach-loading Kerrison 1900. Thus, the bag or balloon can be loaded onto the Kerrison 1900 in a relatively quick manner by loading the bag or balloon into the rear portion of the Kerrison 1900. In some embodiments, the actuation element 1916 is a bellows (e.g., stainless steel metal bellows such as those manufactured by BellowsTech, Inc.). The actuation element 1916 can be configured to exert an axial force on the piston 1920 (e.g., a force upon the first guide portion 1921a) to move the piston 1920 in the distal axial direction. The Kerrison 1900 can include a biasing structure 1924 (e.g., a spring or other resilient structure) configured to bias the piston 1920 in the proximal axial direction. For example, the biasing structure 1924 can provide a return force to return the piston 1920 to push the piston 1920 in the proximal axial direction when the axial force from the actuation element 1916 is reduced and/or removed.

In some embodiments, the Kerrison 1900 includes a return valve (not shown) configured to introduce physiological saline so as to provide compression to the actuation element 1916. The return valve may, for example, allow injection of pressurized gas into the distal actuation chamber 1917 or in the region of the proximal actuation chamber 1919 forward the first guide portion 1921a. The fluid introduced via the return valve can be used to move the piston 1920 in the proximal direction. In some such embodiments, the Kerrison 1900 does not include a biasing structure 1924.

The actuation element 1916 can be fluidly connected to a conduit 1914 through which physiological saline can be input into and pulled out from the actuation element 1916. In some embodiments, hydraulic controls associated with the actuation element 1916 are operated via a foot pedal. Such embodiments can allow for greater dexterity for the user of the Kerrison 1900 by reducing the operating variables controlled by the Kerrison 1900 handle portions 1918, 1923. Elastomeric and/or proportional valves can be used to enhance the responsiveness of the Kerrison 1900 to operation of a foot pedal.

As another example, a pneumatically-driven Kerrison can be used. In some embodiments, the Kerrison can be driven by fluid (e.g., hydraulic of pneumatic) by a bellows actuators.

In the tool embodiments disclosed herein, including without limitation the Kerrison, the cutting surface or top surface of the tool can be bayonetted. The bayonetted structure of the tool can allow the tool to be inserted into the surgical area without interfering or obscuring the views of the surgical site or overhead views of the surgical field. The bayonet style tool can be utilized for the Kerrison, forceps, scissors, or other tools described herein. The bayonet configuration can be advantageous for small surgical sites or external viewing of the surgical site. The bayonet feature reduces the area obscured by the tool within the surgical site.

In any of the tool embodiments disclosed herein, including without limitation the Kerrison, the housing supporting or comprising the tool can be configured to have a port or lumen therein arranged to facilitate the removal of tissue and bone extracted from the surgical site. For example, the Kerrison can have a side port or opening located proximal of the cutting head, though which cut tissue can be removed (e.g., pushed through port or opening as cutter withdraws and Kerrison returns to the default position). In some embodiments, a source of suction, or a source of saline and suction, can be supplied to the port. Additionally, the removal port or lumen of the housing can also support a mechanical removal mechanism, such as but not limited to a screw type auger (which can be hydraulically actuated, via for example a gear motor, gerotor, or vane motor), to facilitate removal of bone debris and extracted tissue from the surgical site. In some embodiments, the removed tissue can be extracted to a waste reservoir supported by or tethered to the housing of the tool. In another embodiment, the movable cutting head of the Kerrison can be a generally cylindrical tube that can be actuated (in the matter described above) to slidably move against the fixed cutting surface 1730. For example, said cylindrical tube can be slidable within an outer housing of the Kerrison when a force is exerted thereon via the expansion of the second inflatable element.

Additionally, in any of the tool embodiments disclosed herein, including without limitation the Kerrison, the housing supporting or comprising the tool can be configured to have a suction port and a source of saline so that the tool and/or the surgical site can be flushed with saline and the saline and debris can removed via the suction line simultaneously or sequentially with the flushing. In some embodiments, the saline can be provided through the conduit used to provide saline to the second inflatable element, through the same or a different lumen of such conduit.

Additionally, the saline source or conduit and/or the suction source or conduit can be separate from the tool so that it can be independently positioned. In some embodiments, the saline source or conduit and/or the suction source or conduit can be tethered to the tool.

Any of the hydraulic system or pneumatic system embodiments disclosed herein can be configured to incorporate or use any suitable surgical tools, including without limitation scissors, micro-scissors, forceps, micro-forceps, bipolar forceps, clip appliers including aneurysm clip appliers, rongeur, and, as described, Kerrison tools.

### Turbine

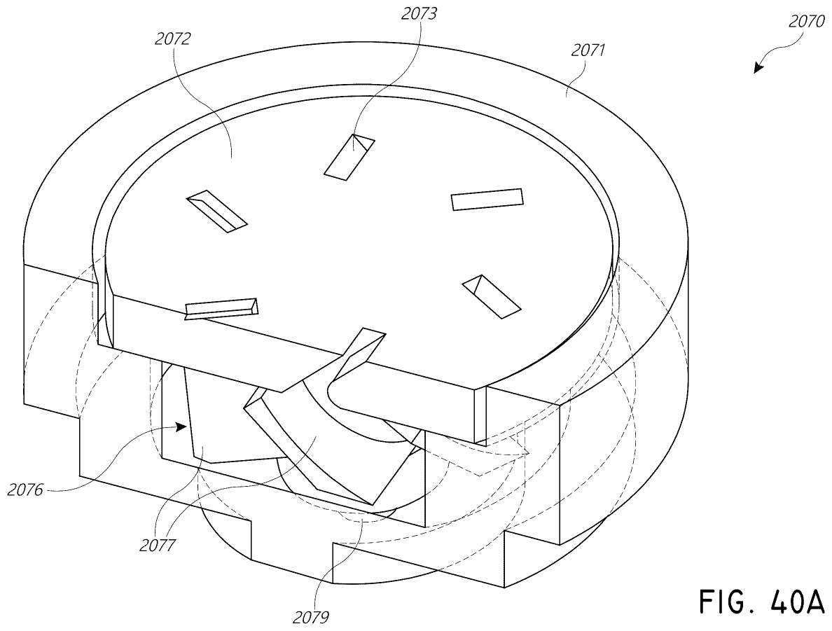

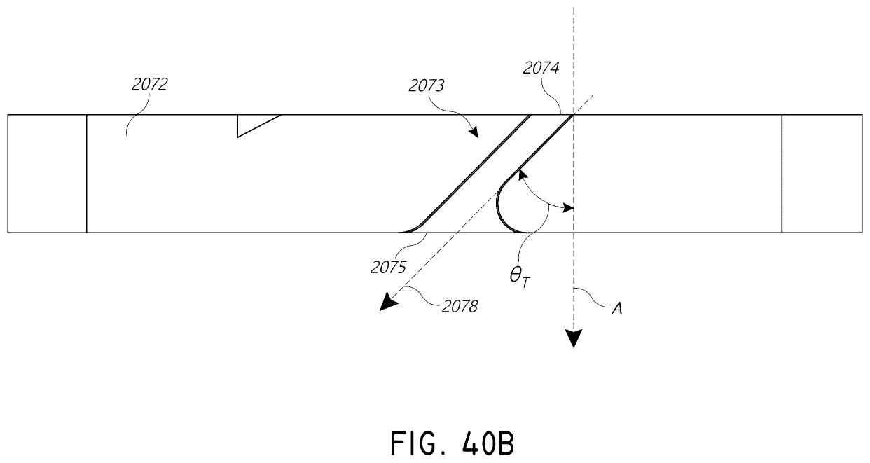

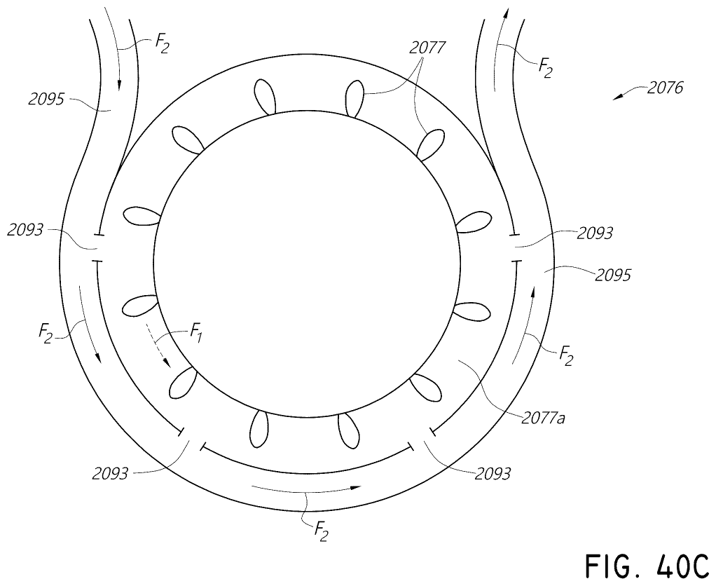

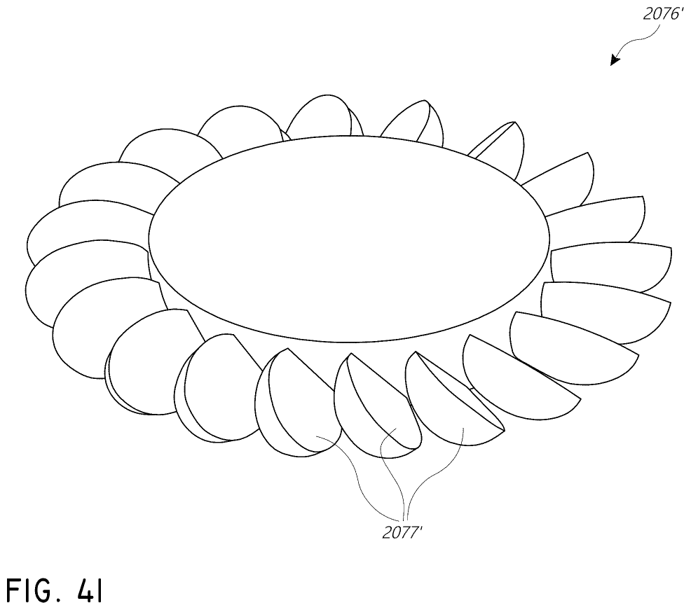

Another tool is a drill which can be driven by a hydraulic turbine. The tool can be driven by a hydraulic turbine. In addition, in some embodiments the drill can be pneumatically powered. In some embodiments, as illustrated in FIGS. 40A and 40B, a hydraulic turbine 2070 includes a turbine housing 2071. In some cases, at least a portion of a nozzle frame 2072 is housed within the turbine housing 2071. In some embodiments, stator vanes can be used in conjunction with and/or in place of the nozzle frame 2072. The nozzle frame 2072 can include one or more turbine nozzles 2073. In some embodiments, the turbine nozzles 2073 are positioned in a circumferential array, as illustrated in FIG. 40A. Each of the turbine nozzles 2073 can have a nozzle inlet 2074 and a nozzle outlet 2075. In some embodiments, the nozzles 2073 have substantially constant cross-sectional areas from nozzle inlet 2074 to nozzle outlet 2075 (e.g., drill hole-type nozzles). For example, circular nozzles can be used.

The relative areas of the nozzle inlet 2074 and the nozzle outlet 2075 can vary. For example, the nozzle outlet 2075 can have an area that is greater than or equal to approximately 125% of the area of the nozzle inlet 2074 and/or less than or equal to about 600% of the area of the nozzle inlet 2074. In some embodiments, the area of the nozzle outlet 2075 is approximately 300% of the area of the nozzle inlet 2074.

As illustrated in FIG. 40B, the profile of the nozzle 2073 can widen between the nozzle inlet 2074 and the nozzle outlet 2075. The rate at which the turbine nozzle 2073 widens between the nozzle inlet 2074 and the nozzle outlet 2075 can vary. For example, the nozzle 2073 can flare out in the direction of the nozzle outlet 2075. In some embodiments, the profile of the nozzle 2073 narrows between the nozzle inlet 2074 and the nozzle outlet 2075. In some embodiments, the nozzles 2073 have substantially constant cross-sectional areas from nozzle inlet 2074 to nozzle outlet 2075 (e.g., drill hole-type nozzles). In some embodiments, the nozzle inlet 2074 can be tapered or flared in such that an opening into the nozzle inlets 2074 is wider or larger than a midsection of the nozzles 2073.

In some embodiments, physiological saline is directed through the nozzle frame 2072 toward an impeller 2076. The impeller 2076 can include a plurality of impeller blades 2077 around the outer periphery of the hub of the impeller 2076. The impeller blades 2077 can rotate within a blade cavity 2077a. (See FIG. 40C.) The impeller 2076 can be integral with or otherwise rotationally coupled with an output shaft 2079 for driving the tool 2082, which can be a drill or other rotational tool. The outer diameter of the hub of the impeller 2076 can be smaller than the outside diameter of the array of hydraulic nozzles 2073. For example, the outer diameter of the hub of the impeller 2076 can be greater than or equal to approximately 15% of the outer diameter of the hydraulic nozzles 2073 and/or less than or equal to approximately 75% of the outer diameter of the hydraulic nozzles 2073. In some cases, the outer diameter of the impeller 2076 can be greater than or equal to 0.5 inches and/or less than or equal to approximately 1.5 inches. Many variations sizes and relative sizes of the components of the hydraulic turbine 2070 and its subcomponents are possible.

In some cases, the impeller blades 2077 are oriented at an angle offset from the central axis of the impeller 2077. The hydraulic nozzles 2073 can be configured to turn the flow of physiological saline from an axial direction A to nozzle direction 2078 as the flow is passed through the nozzle frame 2072 toward the impeller 2076. The nozzle direction 2078 can be selected to be at an angle θᵣ offset from axial A such that the nozzle direction 2078 is substantially perpendicular to the faces of the impeller blades 2077. The closer nozzle outlets are to the plane of the impeller blades 2077 and the more radially-directed the flow from the nozzles, the more torque can be imparted upon the impeller blades 2077. For example, the nozzle outlets can be positioned close to the impeller blades 2077 in the axial direction and can direct physiological saline at a highly-radial angle toward impeller blades 2077 whose surfaces are close to parallel to the rotation of axis of the impeller 2076.

In some cases, utilizing a plurality of circumferentially-distributed turbine nozzles 2073 to drive a plurality of impeller blades 2077 can increase the torque output of impeller 2076 as compared to a configuration wherein only one turbine nozzle 2073 is utilized. In some such configurations, the outer diameters of the nozzle frame 2072 and impeller 2076 can smaller than a single-nozzle configuration of equal output torque.

In some embodiments, the hydraulic turbine 2070 can be configured to operate at rotational speeds of 40,000 rpm to 60,000 rpm, though higher and lower rpm values may be possible. In some embodiments, the hydraulic turbine is configured to operate at rotational speeds of 100,000 rpm. The hydraulic turbine 2070 can be configured to operate at operating pressures between 70 psi and 190 psi, though greater and lesser operating pressures are possible. In some embodiments, the operating pressure of the hydraulic turbine 2070 is designed to be approximately 120 psi.

As illustrated in FIG. 41, an impeller 2076′ can be designed to have bucket-shaped impeller blades 2076′. The bucket-shaped impeller blades 2077′ can be oriented at an angle of approximately 45° from the axial direction A. Many variations of the impeller blade 2077′ angles are possible. Additionally, many different shapes of blades 2077 are possible, such as Pelton or Turgo shaped blades.

As illustrated in FIG. 40C, the hydraulic turbine 2070 can be designed to collect the physiological saline that has already impacted the impeller blades 2077, 2077′ (hereinafter 2077 for simplicity). For example, an exhaust angle can be calculated to represent the angle at which physiological saline reflects off of the impeller blades 2077 after impact with the impeller blades 2077. One or more vacuum ports 2093 can be positioned on or in the turbine housing 2071 to extract the fluid F1 that is reflected off of the impeller blades 2077 and redirect the fluid F1 into a bypass channel 2095. In some embodiments, the vacuum source can be an external pump (e.g., a peristaltic pump) or the vacuum can be the result of a Venturi effect created by the diversion of fluid. For example, in some embodiments, the vacuum source can be provided by diverted, high velocity fluid F2 directed to bypass the impeller 2076. The high velocity fluid F2 may be hydraulic or pneumatic. In some embodiments, the high velocity fluid F2 may be provided by a hospital line. In other embodiments, the high velocity fluid F2 may be provided by a source other than a hospital line. In some embodiments, one or more ports 2093 in the hydraulic turbine housing 2071 (e.g., on the side of the housing closer to the impeller 2076 than to the nozzle frame 2072) can create fluid communication between the reflected fluid F1 in the blade cavity 2077A and the diverted high velocity fluid F2 in the bypass channel 2095. The pressure differential between the two fluid bodies (e.g., lower pressure in fluid F2 and higher pressure in fluid F1) will pull the reflected fluid F1 out of the housing 2071 and into the diverted fluid path 2095. Removal of the reflected fluid from the housing 2071 can increase the performance of the turbine 2070 by reducing the viscous drag on the impeller from undiverted fluid F1. For example, the viscous frictional losses that would be otherwise incurred from interaction between the reflected fluid F1 and the impeller 2076 and/or output shaft 2079 can be reduced. The diverted high velocity fluid F2 and scavenged reflected fluid F1 can be diverted back to the cassette 2020 for re-pressurization. In some embodiments, scavenging reflected fluid F1 and diverting it back to the cassette 2020 can reduce the amount of physiological saline required to operate the tools and/or other components of the system. In some embodiments, the housing 2071 can include one or more ports open to ambient. Such ports can be configured to receive pressurized air or other pneumatic gas. In some embodiments, the turbine 2070 is configured to operate as a dual hydro/pneumatic turbine configured to operate via hydraulic power and pneumatic power or a continual variance of hydraulic and pneumatic power. A controller or switch(s) can be used to vary the amount of hydraulic fluid or pneumatic air or gas are applied to the turbine. In some embodiments, the controller or switch(es) allow the user to increase pneumatic gas or air and decrease hydraulic or vice versa. The pneumatic gas or air and hydraulic fluid can be provided by output ports on the display console. Likewise, the controller and/or switch(es) may be on the controller or remotely located.

In some embodiments, multiple impellers 2076 (e.g., multiple turbine wheels) can be utilized in the same turbine housing 2071. In some such embodiments, the overall diameter of the turbine 2070 and/or some of its components can be reduced relative to a single-impeller turbine 2070 without sacrificing output torque.

In some embodiments, actuation of the turbine 2070 can be controlled via a foot pedal. In some embodiments, depressing the foot pedal applies a vacuum source to the turbine 2070, thus causing the turbine 2070 to be scavenged, before any fluid (e.g., pressurized saline) is delivered to drive the turbine 2070. In some embodiments, a first depression of the foot pedal scavenges the turbine 2070, while a second depression of the foot pedal delivers fluid to the turbine 2070 and activates the turbine 2070. In other embodiments, one depression of the foot pedal scavenges the turbine 2070 and also delivers fluid to the turbine 2070 to drive the turbine 2070 after the turbine is scavenged.

In some embodiments, the turbine 2070 is configured to operate as a dual hydro/pneumatic turbine configured to operate via hydraulic power and pneumatic power or a continual variance of hydraulic and pneumatic power. The percentage of liquid and the percentage of air used to drive the dual hydro/pneumatic turbine can vary. In addition, the percentage of liquid and the percentage of air used to drive the dual hydro/pneumatic turbine can be controlled by an operator. The hydro/pneumatic turbine may operate with 100% liquid, 100% air, or any combination in between. As an illustrative example, the hydro/pneumatic turbine may operate with 10% liquid and 90% air; 20% liquid and 80% air; 30% liquid and 70% air; 40% liquid and 60% air; 50% liquid and 50% air; 60% liquid and 40% air; 70% liquid and 30% air; 80% liquid and 20% air; 90% liquid and 10% air; or a combination in between any of these values. The percentage of liquid and the percentage of air used to drive the turbine may vary at any given time. As an illustrative example, 100% liquid may be used to begin driving the turbine, then the percentage of liquid used to drive the turbine may gradually decrease while the percentage of air used to drive the turbine may gradually increase, until the turbine is driven with 100% air. In some embodiments, driving the turbine with more liquid causes the turbine to operate with more torque. Thus, for example, the turbine may operate with the most torque when driven with 100% liquid. In some embodiments, driving the turbine with more air causes the turbine to operate with more speed. Thus, for example, the turbine may operate with the most speed when driven with 100% air.

Some instruments such as surgical tools use torque or mechanical force to translate manual input into tool actuation. For example, a Kerrison for bone removal generally includes a handle mechanically coupled to a head including a stationary portion and a movable portion. When a user squeezes the handle, the movable portion moves closer to the stationary portion in a cutting manner (e.g., in a shearing manner), for example to remove bone by trapping the removed bone between the stationary portion and the movable portion (e.g., within a channel between the stationary portion and the movable portion). Other examples of tools include an aneurysm clipper, a rongeur, forceps, scissors, and the like, although many other hand-operated tools are known to those skilled in the art. Referring again to the Kerrison, the pace and force of the squeezing translates to the pace and force of the cutting, and this phenomenon is also applicable to other hand-operated tools. This translation can be disadvantageous, for example varying based on each user, being too slow or too fast or having variable speed, lacking force or imparting too much force or having variable force, etc. Additionally, periodic use of such manually operated tools (e.g., during a lengthy operation or procedure) can lead to hand fatigue of the surgeon or user. Manual actuation leads to inadvertent movement of the tool tip. In some embodiments, planetary gearhead can be utilized to slow down the drill and/or increase torque.

The hydraulic system may also be used for other purpose such as cleaning optics and/or cooling light emitters for illuminating a surgical site.

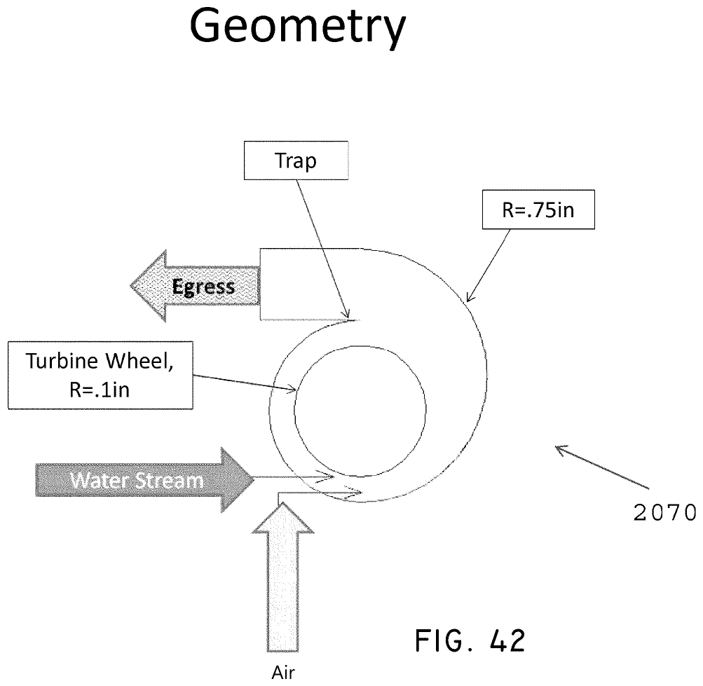

As discussed above, the hydraulic system may be used to drive hydraulic tools such as a hydraulic drill or other tool having an actively and/or passively scavenged hydraulic turbine. As illustrated in FIG. 42, in some embodiments the hydraulic turbine 2070 can include a liquid (e.g., water or saline) stream ingress and an air ingress (e.g., the arrows in the bottom left of FIG. 42). The hydraulic turbine 2070 also include a fluid egress port. In some embodiments, the radius of the turbine wheel can be approximately 0.1 inches. In some embodiments the radius of the turbine wheel is greater than about 0.02 inches and/or less than about 4 inches. Many variations are possible. The liquid can be directed onto the paddles or buckets of the hydraulic turbine at a radius Rp. A lower limit for stream velocity (e.g., the velocity of the liquid as it deflects from the paddles) of the liquid when the paddle wheel is turning at a substantially constant rate can be, for example, the tangential velocity of the paddle wheel at the point of contact between the liquid and the paddle wheel (e.g., when there is little or no kinetic energy transfer between the liquid and the wheel). The stream velocity can be calculated by multiplying the radius of the contact point between the liquid and the paddle with the rpm of the paddles and 2π.

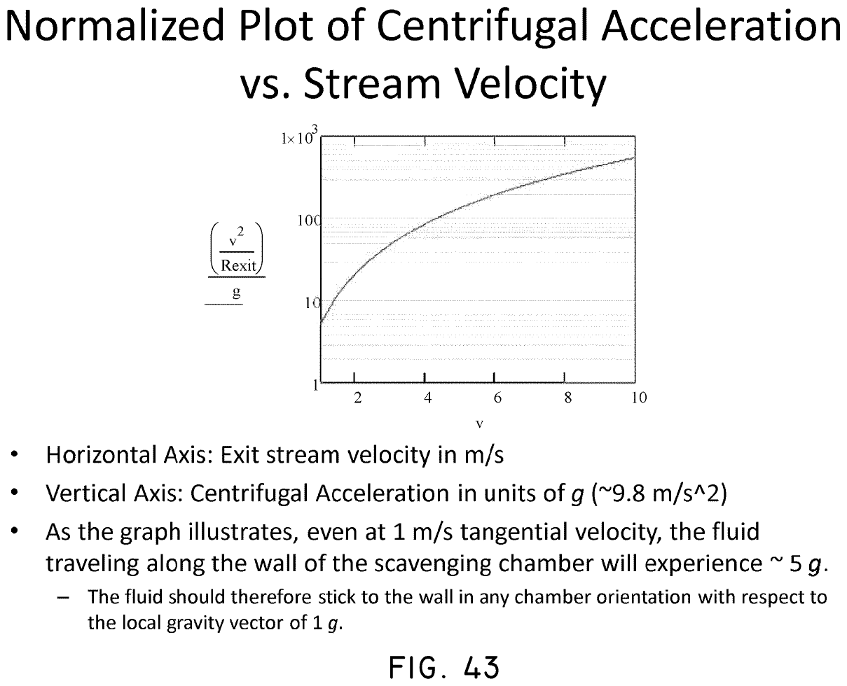

The centrifugal acceleration of the exit stream (e.g., the stream of fluid that exits through the fluid egress) can be estimated as the exit velocity squared divided by the radius of the egress Rexit. Thus, Rp and Rexit can be proportional. Rexit can be approximately 0.75 inches. In some embodiments, the Rexit is greater than about 0.25 inches and/or less than or equal to about 8 inches. In some embodiments, Rexit is greater than about 125% of Rp and/or less than or equal to about 1000% of Rp. Many variations in the value of Rexit are possible. As illustrated in FIG. 43, centrifugal acceleration of the fluid within the turbine 2070 can be greater than 1 g (e.g., greater than the local gravity vector). In some such configurations, the fluid in the turbine 2070 should “stick” to the wall of the turbine housing after deflecting from the paddles.

In some embodiments, the turbine 2070 has a single, large egress port. Having a single large egress port can reduce the likelihood that capillary forces would block the egress port. In some embodiments, using a single egress port can increase the likelihood that both liquid and air exit through the egress port. Using a single large egress port can reduce or minimize egress pathway surface area. Vortex scavenging can utilize the kinetic energy of the liquid stream to keep the liquid away from the paddles of the turbine after initial contact with the paddles. In some embodiments, using vortex scavenging can reduce or minimize restrictions for the egressing air/liquid mixture. Generation of centrifugal forces (e.g., forces greater than 1 g) can help to keep fluid at the outer wall of the turbine chamber after deflection of the fluid from the paddles. In some embodiments, centrifugal forces push the denser liquid (e.g., denser than air) toward the walls to encourage egress of the liquid after incidence with the paddles.

In various embodiments, to drive hydraulic and pneumatic tools such as described above, the console can be equipped with a hydraulic and/or pneumatic system. An example of such a hydraulic and/or pneumatic system may include, for example, one or more pneumatic actuator chambers having pressurized physiological saline or other hydraulic fluid therein that can be used to drive tools (e.g., kerrison, scissors) configured to be driven hydraulically. In various embodiments, a compressed gas source (e.g., a hospital compressed gas source, such as compressed nitrogen tanks) can be used to pressurize the physiological saline within the pneumatic actuator chambers. In certain embodiments, dual chamber pneumatic actuators that alternated are used to provide more continuous hydraulic pressure. Such a hydraulic system is discussed in U.S. patent application Ser. No. 14/283,106 (Attorney Docket No. CAMPLX.039A), which is incorporated herein by reference in its entirety. In some embodiments, the fluid reservoir (e.g., an IV bag) is connected to the pneumatic actuator chambers to maintain a sufficient level of hydraulic fluid therein. In some embodiments, the compressed gas source pressurizes the physiological saline within the fluid reservoir. In such configurations, a reservoir pressurization line connects the compressed gas source (e.g., hospital compressed gas system) to the fluid reservoir (e.g., an IV bag). In some embodiments, the fluid reservoir is fluidly connected to one or more fluid outlets (e.g., nozzles) configured to wash optical components (e.g., cameras, LEDs, and/or other components). One or more valves (e.g., elastomeric and/or proportional valves) can be positioned in pathways to control the flow of the hydraulic fluid from the chambers. Such valves may be included in one or more cassette. Cassettes are discussed in U.S. patent application Ser. No. 14/283,106 (Attorney Docket No. CAMPLX.039A), which is incorporated herein by reference in its entirety.

In certain embodiments, a vacuum source (e.g., a hospital vacuum source) can be fluidly connected to one or more components of the hydraulic pressure system. For example, the vacuum source can be connected to one or more of the actuator chambers. Filters (e.g., hydrophobic and/or anti-microbial filters) can be positioned in the fluid paths between the actuator chambers and the vacuum source. In some embodiments, the filters are configured to reduce the likelihood that contaminants from within the hydraulic pressure circuit are introduced to the hospital vacuum source.

One or more valves (e.g., elastomeric and/or proportional valves) can be positioned on the fluid lines between the vacuum source and the actuator chambers. Such valves may be included in one or more cassettes as discussed in U.S. Patent Application Ser. No. 14/283,106 (Attorney Docket No. CAMPLX.039A), which is incorporated herein by reference in its entirety. The valves can be configured to selectively permit fluid communication between the vacuum source and the actuator chambers. In some embodiments, the valves can be configured to be operated by user input (e.g., a foot pedal) to open/close in a proportional manner. Accordingly, the actuator chambers can receive input both from a pressure source (e.g., hospital pressure) and a vacuum source (e.g., hospital vacuum). Input from both sources may provide more precise and responsive control of pressure within the actuator chambers. As an example, a proportional-integral-derivative controller (PID controller) may be utilized to sense the pressure and/or vacuum level and provide feedback so that the pressure and/or vacuum inputs may be adjusted according to the desired setpoints. Proportional operation of the valves can also enhance the precision with which a user of the hydraulic pressure circuit can regulate the hydraulic fluid pressure within the actuator chambers. Such precision can be useful for controlling surgical tools and other surgical equipment especially in delicate procedures that require extreme precisions like neurosurgery. In some embodiments, fluid communication between the vacuum source and the actuator chambers can enable accelerated filling of the actuator chambers with physiological saline.

The hydraulic/pneumatic pressure system can also drive tools (e.g., bi polar forceps, kerrison, scissors) configured to operate via pneumatic pressure. The pneumatic pressure can be provided by a pump (e.g., a 60 psi air pump). In some embodiments, the tool is pneumatically powered via a pressure source such as a hospital compress gas source. For example, the tool can be fluidly connected to the pneumatic pressure source via the hydraulic manifold and/or via one or more valves (e.g., proportional elastomeric valves). Such valves may be included in a cassette. Cassettes are discussed in U.S. patent application Ser. No. 14/283,106 (CAMPLX.039A), which is incorporated herein by reference in its entirety.

### Example Numbered Embodiments

The following is a list of some example numbered embodiments. The examples presented herein are not intended to limit the scope of the disclosed embodiments, but merely represent exemplary combinations to illustrate potential uses and configurations. Nothing in the following should be interpreted to indicate that any one piece or component is essential to the embodiments disclosed herein.

### Offset Image Sensors Facing One Another

1. A stereo camera system comprising:
a pair of image sensors comprising a left image sensor and a right image sensor, each of the pair of image sensors having an active detection area on a front face of the image sensor, the left image sensor being offset along a first direction from the right image sensor, the front face of the left image sensor oriented such that a plane of the front face of the left image sensor is parallel to a plane of the front face of the right image sensor, the front face of the left image sensor facing the front face of the right image sensor, each of the planes of the front faces oriented perpendicular to the first direction;
a pair of lens trains comprising a left lens train having a plurality of lens elements along a left optical path and a right lens train having a plurality of lens elements along a right optical path, the left optical path offset along the first direction from the right optical path; and
a pair of optical redirection elements comprising a left optical redirection element positioned along the left optical path and configured to redirect the left optical path to the front face of the left image sensor and a right optical redirection element positioned along the right optical path and configured to redirect the right optical path to the front face of the right image sensor.

2. The stereo camera system of Embodiment 33, wherein the left optical redirection element comprises a left prism and the right redirection element comprises a right prism.

3. The stereo camera system of Embodiment 34, wherein the left prism is offset from the right prism along the first direction.

4. The stereo camera system of Embodiment 34, wherein the left prism comprises a primary reflective face that is orthogonal to a primary reflective face of the right prism.

5. The stereo camera system of Embodiment 33, wherein the left optical redirection element comprises a left mirror and the right redirection element comprises a right mirror.

6. The stereo camera system of Embodiment 33, wherein the left optical redirection element is configured to redirect the left optical path 90 degrees and the right optical redirection element is configured to redirect the right optical path 90 degrees, the redirected left optical path and the redirected right optical path being anti-parallel to one another.

7. The stereo camera system of Embodiment 33, wherein the left optical path and the right optical path are parallel.

8. The stereo camera system of Embodiment 33, wherein the left image sensor is a two-dimensional detector array and the right image sensor is a two-dimensional detector array.

9. The stereo camera system of Embodiment 40, wherein the left image sensor is a CCD detector array and the right image sensor is a CCD detector array.

10. A surgical visualization system comprising a plurality of camera systems, at least one of the plurality of camera systems comprising the stereo camera system of Embodiment 151.

11. A retractor including the stereo camera system of Embodiment 33 disposed thereon.

12. A surgical tool including the stereo camera system of Embodiment 33 disposed thereon.

13. A stereo camera system comprising:
a pair of image sensors comprising a left image sensor and a right image sensor;
a pair of lens trains comprising a left lens train having a plurality of lens elements along a left optical path and a right lens train having a plurality of lens elements along a right optical path, the left optical path offset laterally from the right optical path; and
a pair of optical redirection elements comprising a left optical redirection element positioned along the left optical path and configured to redirect the left optical path to the front face of the left image sensor and a right optical redirection element positioned along the right optical path and configured to redirect the right optical path to the front face of the right image sensor.

14. A surgical tool including the stereo camera system of Embodiment 45 disposed thereon.

15. A retractor including the stereo camera system of Embodiment 45 disposed thereon.

Display Enclosure Assembly Separate from Binocular Assembly

1. A medical apparatus comprising:
one or more electronic displays comprising a plurality of pixels configured to produce a two-dimensional image;
first and second imaging optics disposed respectively in first and second optical paths from said one or more electronic displays to form respective first and second collimated optical beams and images disposed at infinity;
a primary housing at least partially enclosing said displays and said imaging optics;
wherein said first and second imaging optics are configured to direct said first and second beams such that the beams are substantially parallel to each other and have cross-sections with centers separated from each other by between about 22 mm and 25 mm
(in some embodiments, the housing can include an opening, and the first and second imaging optics can be configured to direct the first and second beams through the opening, and the first and second beams can be substantially parallel to each other at the opening).

2. The medical apparatus of Embodiment 1, wherein said one or more electronic displays comprise first and second electronic displays.

3. The medical apparatus of Embodiment 1, wherein said one or more electronic displays comprise first and second portions of a single electronic display.

4. The medical apparatus of Embodiment 1, wherein said one or more electronic displays comprise at least one emissive display or at least one spatial light modulator.

5. The medical apparatus of Embodiment 1, wherein said one or more electronic displays comprise at least one liquid crystal display or at least one light emitting diode display.

6. The medical apparatus of Embodiment 1, further comprising a plurality of reflective surfaces in the optical paths of the optical beams to fold the optical beams.

7. The medical apparatus of Embodiment 6, wherein said plurality of reflective surfaces comprise mirrors.

8. The medical apparatus of Embodiment 6, wherein said plurality of reflective surfaces comprise between about 2 and 6 mirrors per side of said reflective surfaces in each of said first and second optical paths.

9. The medical apparatus of Embodiment 1, further comprising a plurality of reflective surfaces to fold the optical beams.

10. The medical apparatus of Embodiment 1, further comprising at least one mirror between said one or more electronic displays and said imaging optics.

11. The medical apparatus of Embodiment 1, further comprising between 0 and 2 mirrors between said one or more electronic displays and said imaging optics.

12. The medical apparatus of Embodiment 1, wherein said imaging optics comprise a plurality of lenses.

13. The medical apparatus of Embodiment 12, further comprising at least one mirror between said imaging optics and an exit pupil of said imaging optics.

14. The medical apparatus of Embodiment 13, wherein said at least one mirror comprises at least one mirror between said electronic display and said imaging optics, and at least one mirror disposed between lenses in said imaging optics, said former mirror being larger than said latter mirror.

15. The medical apparatus of Embodiment 1, wherein said imaging optics comprise between about 2 and 11 lenses.

16. The medical apparatus of Embodiment 1, wherein said imaging optics comprise positive lenses.

17. The medical apparatus of Embodiment 1, wherein said imaging optics have positive power.

18. The medical apparatus of Embodiment 1, wherein said imaging optics comprise a first lens configured to reduce a cross-section of the first beam.

19. The medical apparatus of Embodiment 18, wherein said first lens is configured to substantially collimate said first beam.

20. The medical apparatus of Embodiment 18, wherein said first lens has positive power.

21. The medical apparatus of Embodiment 18, wherein a lens in said imaging optics other than said first lens has an aperture size smaller than said first lens.

22. The medical apparatus of Embodiment 18, wherein lenses in said imaging optics other than said first lens have aperture sizes smaller than said first lens.

23. The medical apparatus of Embodiment 1, wherein said imaging optics is configured to magnify the display.

24. The medical apparatus of Embodiment 1, wherein said imaging optics has a field of view between about 3-10°.

25. The medical apparatus of Embodiment 1, wherein said first and second imaging optics have exit pupils and said first and second electronic displays are not parallel to said exit pupils.

26. The medical apparatus of Embodiment 1, wherein said first and second imaging optics have exit pupils having centers and said first and second electronic displays have centers, said center of said exit pupils being displaced from said centers of said electronic displays.

27. The medical apparatus of Embodiment 1, wherein said first and second imaging optics have exit pupils and an optical path length from said one or more electronic displays to said exit pupils is between about 2 mm and 7 mm.

28. The medical apparatus of Embodiment 1, wherein an optical path length from said one or more electronic displays to said imaging optics is between about 100 mm and 400 mm.

29. The medical apparatus of Embodiment 1, wherein the imaging optics comprise a plurality of lenses including a first lens and a last lens in said optical paths and said imaging optics has an optical path length from said first lens to the last lens that is between about 50 mm and 250 mm.

30. The medical apparatus of Embodiment 1, wherein the imaging optics comprise a plurality of lenses including a first lens and an exit pupil in said optical paths and said imaging optics has an optical path length from said first lens to the exit pupil that is between about 10 mm and 50 mm.

31. The medical apparatus of Embodiment 1, wherein said one or more electronic displays comprise first and second electronic displays having centers spaced apart by a distance Wdisplay, wherein said first and second imaging optics have exit pupils having centers spaced apart by a distance Weye paths, and wherein Wdisplay>Weye paths.

32. The medical apparatus of Embodiment 1, wherein said one or more electronic displays comprise first and second electronic displays having centers spaced apart by a distance between about 100 mm and 200 mm.

33. The medical apparatus of Embodiment 1, wherein said first and second imaging optics have exit pupils having centers spaced apart by a distance of between about 22 mm and 25 mm.

34. The medical apparatus of Embodiment 1, wherein said first and second imaging optics have exit pupils having centers spaced apart by a distance of between about 50 mm and 200 mm over most of a distance through the imaging optics.

35. The medical apparatus of Embodiment 1, wherein said first and second imaging optics have optical axes spaced apart over most of a distance through the imaging optics by a distance of between about 50 mm and 200 mm.

36. The medical apparatus of Embodiment 1, wherein said first and second beams have cross-sections having centers spaced apart over most of a distance through the imaging optics by a distance of between about 15 mm and 35 mm.

37. The medical apparatus of Embodiment 1, wherein said first and second imaging optics have first and second exit pupils disposed a longitudinal distance along the length of the beam that is between about 0 mm and 45 mm from the opening.

38. The medical apparatus of Embodiment 1, wherein said housing has internal sidewalls darker than external sidewalls.

39. The medical apparatus of Embodiment 1, wherein said housing has dark internal sidewalls.

40. The medical apparatus of Embodiment 1, wherein said housing has black internal sidewalls.

41. The medical apparatus of Embodiment 1, further comprising baffles in said housing for reducing stray light.

42. The medical apparatus of Embodiment 1, wherein said opening comprises a mounting face configured to connect to a binocular assembly.

43. The medical apparatus of Embodiment 1, wherein said opening is between about 50 mm and 100 mm wide.

44. The medical apparatus of Embodiment 1, wherein said opening is circular.

45. The medical apparatus of Embodiment 1, wherein said opening is between about 66 mm and 70 mm in diameter.

46. The medical apparatus of Embodiment 1, wherein said opening comprises a mounting face having a size and shape configured to mate with a binocular assembly for a surgical microscope.

47. The medical apparatus of Embodiment 1, further comprising a binocular assembly comprising first and second objectives, first and second beam positioning optics, and first and second oculars.

48. The medical apparatus of Embodiment 47, wherein said binocular assembly has a magnification of between 8× and 13×.

49. The medical apparatus of Embodiment 47, wherein said binocular assembly has a magnification of between 10× and 12.5×.

50. The medical apparatus of Embodiment 47, wherein said imaging optics and said binocular assembly provide an apparent field of view of between 100-120º.

51. The medical apparatus of Embodiment 47, wherein said imaging optics and said binocular assembly provide an apparent field of view of about 110°.

52. The medical apparatus of Embodiment 47, wherein said imaging optics and said binocular assembly provide an apparent field of view of between 60-70º.

53. The medical apparatus of Embodiment 47, wherein said first and second beam positioning optics comprise prisms.

54. The medical apparatus of Embodiment 47, wherein said imaging optics have exit pupils having centers and said binocular assembly have entrance pupils having centers, and said centers of said entrance pupils are separated by a distance that is substantially the same as the separation between said centers of said exit pupils.

55. The medical apparatus of Embodiment 47, wherein said binocular assembly has entrance pupils having centers, and said centers of said entrance pupils are separated by a distance of between about 52 mm and 78 mm.

56. The medical apparatus of Embodiment 47, wherein said imaging optics have exit pupils and said binocular assembly have entrance pupils, and said entrance pupils are smaller than said exit pupils.

57. The medical apparatus of Embodiment 47, wherein said imaging optics have exit pupils and said binocular assembly have entrance pupils, and said entrance pupils are the same size as said exit pupils.

58. The medical apparatus of Embodiment 47, wherein said binocular assembly has entrance pupils, and said entrance pupils are 15 mm to 20 mm in diameter.

59. The medical apparatus of Embodiment 47, wherein said oculars on said binocular assembly have adjustable tilt to accommodate different heights of surgeons.

60. The medical apparatus of Embodiment 47, wherein said binocular assembly has a housing with an opening and said opening is configured to interface with and connect to the opening of said primary housing.

61. The medical apparatus of Embodiment 47, wherein said imaging optics have exit pupils disposed in an exit pupil plane and said binocular assembly have entrance pupils disposed in an entrance pupil plane, and said entrance pupil plane and said exit pupil plane are substantially coplanar.

62. The medical apparatus of Embodiment 47, wherein said entrance pupil plane and said exit pupil plane are separated by less than about 0 mm to 30 mm.

63. The medical apparatus of Embodiment 62, wherein said entrance pupil plane and said exit pupil plane are separated by less than 0 mm to 15 mm.

64. The medical apparatus of Embodiment 1, further comprising an articulated arm supporting said primary housing of said one or more electronic displays.

65. The medical apparatus of Embodiment 1, further comprising processing electronics configured to communicate with said one or more electronic displays to provide images for said one or more electronic displays.

66. The medical apparatus of Embodiment 65, wherein said electronics is configured to receive images from one or more cameras on a surgical device.

67. The medical apparatus of Embodiment 65, wherein said electronics is configured to receive images from one or more cameras on a surgical tool.

68. The medical apparatus of Embodiment 65, wherein said electronics is configured to receive images from one or more cameras on a surgical retractor.

69. The medical apparatus of Embodiment 65, wherein said electronics is configured to receive images from one or more cameras that provide a surgical microscope view.

70. The medical apparatus of Embodiment 69, wherein said camera is supported by an articulated arm that supports said primary housing for said electronic displays.

71. The medical apparatus of Embodiment 69, wherein said primary housing for said displays is supported by an articulated arm and said one or more cameras that provide a surgical view are supported by a separate platform that is configured to be able to be stationary with movement of said articulated arm.

72. The medical apparatus of Embodiment 65, wherein said first and second displays receive input images from said processing electronics corresponding respectively to left and right channels on a stereo camera and display said input images on said first and second electronic displays respectively.

73. The medical apparatus of Embodiment 72, further comprising a binocular assembly that receives said first and second beams from said first and second imaging optics and has oculars that output images from said first and second electronics display so as to render a three-dimensional image visible to a viewer peering through said oculars.

74. The medical apparatus of Embodiment 65, wherein said processing electronics are configured to receive images from memory that store previously recorded images.

75. The medical apparatus of Embodiment 65, wherein said processing electronics are configured to present images sources other than cameras.

76. The medical apparatus of Embodiment 65, wherein said processing electronics are configured to present images sources other than cameras in addition to images from cameras for simultaneous viewing by a viewer.

77. The medical apparatus of Embodiment 75 or 76, wherein said sources of images other than cameras comprises Computer Aided Tomography (CAT) scan, MRI, x-ray, and ultrasound imaging instruments.

78. The medical apparatus of Embodiment 75 or 76, wherein said source comprises a source of artificially generated image data.

79. The medical apparatus of Embodiment 1, further comprising at least one beam splitter disposed in one or both of said first and second optical paths configured to receiving images to be viewable by a binocular assembly connected to said primary housing in addition to images from said electronics displays.

80. The medical apparatus of Embodiment 79, further comprising at least one separate electronic display disposed with respect to said at least one beam splitter such that said one or both of said first and second optical paths receives images produced on said at least one electronic display through said at least one beam splitter for viewing through said binocular assembly connected to said housing in addition so images from said electronics displays.

81. The medical apparatus of Embodiment 80, wherein said at least one beam splitter comprises first and second beam splitters and said at least one separate electronic display comprises first and second displays configured to display a pair of two-dimensional images which together when viewed through said binocular assembly produces a three-dimensional image.

82. The medical apparatus of Embodiment 1, further comprising an assistant display housing containing at least one assistant electronic display and assistant display imaging optics for imaging images produced on said at least one assistant electronic display.

83. The medical apparatus of Embodiment 82, wherein said assistant display housing contains first and second assistant electronic displays and first and second assistant display imaging optics for imaging images produced on said first and second electronic displays.

84. The medical apparatus of Embodiment 82, wherein said primary housing and said assistant housing are supported by a common articulated arm.

85. The medical apparatus of Embodiment 82, wherein said assistant housing and said primary housing are connected via a support post such that said assistant housing can rotate with respect said primary housing.

86. The medical apparatus of Embodiment 82, wherein said assistant housing is configured to rotate with respect to said primary housing without moving said primary housing. 87. The medical apparatus of Embodiment 82, wherein said assistant housing is configured to rotate with respect to said primary housing to accommodate an assistant on opposite side of surgeon facing surgeon.

88. The medical apparatus of Embodiment 82, wherein said assistant housing is configured to rotate with respect to said primary housing to accommodate an assistant on left or right sides of a primary surgeon.

89. The medical apparatus of Embodiment 82, wherein said assistant housing is configured to rotate through at least 180° with respect to said primary housing.

90. The medical apparatus of Embodiment 89, wherein said assistant housing can rotate from +90° with respect to said primary housing to at least 270° with respect to said primary housing.

91. The medical apparatus of Embodiment 82, wherein said assistant housing is smaller than said primary housing.

92. The medical apparatus of Embodiment 82, wherein said at least one electronic display in said assistant housing is smaller than said one or more electronic displays in said primary housing.

93. The medical apparatus of Embodiment 82, wherein said imaging optics in said assistant housing are smaller than said imaging optics in said primary housing.

94. The medical apparatus of Embodiment 82, wherein said primary housing and said assistant housing are stacked, one over the other.

95. The medical apparatus of Embodiment 82, wherein said primary housing is disposed over said assistant housing.

96. The medical apparatus of Embodiment 82, wherein said assistant housing is disposed over said primary housing.

97. The medical apparatus of Embodiment 82, wherein said assistant housing is disposed between said primary housing and a camera that provides surgical microscope views.

98. The medical apparatus of Embodiment 97, wherein said assistant housing is disposed between said primary housing and movable support for said camera that provides surgical microscope views.

99. The medical apparatus of Embodiment 82, wherein first and second optical paths in said assistant display rotate with respect to first and second optical paths of said assistant housing.

100. The medical apparatus of Embodiment 82, wherein said at least one electronic displays in said assistant display rotates with respect to said at least one electronic display of said assistant housing.

101. The medical apparatus of Embodiment 82, further comprising processing electronics in communication with said at least one electronic displays in said assistant display configured to adjust the images presented on said at least one electronic displays in said assistant display based on the orientation of the assistant display housing with respect to the primary housing.

102. The medical apparatus of Embodiment 101, further comprising sensors to determine an orientation of the assistant housing that provides input to said processing electronics to adjust the images presented on said at least one assistant electronic display depending on said orientation.

103. The medical apparatus of Embodiment 102, further comprising at least four cameras for providing a surgical microscope view, said processing electronics selecting images from different pairs of said four cameras depending on said orientation of said assistant housing.

104. The medical apparatus of Embodiment 103, wherein said least four cameras comprise four cameras in a square 2×2 array and said electronics select a pair of said four cameras depending on said orientation of said assistant housing.

105. The medical apparatus of Embodiment 1, wherein said primary housing has a width between about 110 mm and 250 mm.

106. The medical apparatus of Embodiment 82, wherein said assistant housing has a width between about 50 mm and 150 mm.

107. The medical apparatus of Embodiment 1, wherein said primary housing has a length between about 150 mm and 350 mm.

108. The medical apparatus of Embodiment 82, wherein said assistant housing has a length between about 75 mm and 175 mm.

109. The medical apparatus of Embodiment 2, wherein said first and second electronic displays present left and right two-dimensional images having parallax such that a viewer viewing through a binocular assembly receiving light from said imaging optics can see a three-dimensional image.

110. A medical apparatus comprising:
one or more electronic displays comprising a plurality of pixels configured to produce a two-dimensional image;
first and second imaging optics disposed respectively in first and second optical paths from said one or more electronic displays to form respective first and second substantially collimated optical beams;
a primary housing at least partially enclosing said displays and said imaging optics;
an opening in said housing,
wherein said first and second imaging optics are configured to direct said first and second beams through said opening.

### Electronic Display Assembly-Primary and Assistant

1. A medical apparatus comprising:
a primary display assembly; and
an assistant display assembly comprising:
   one or more electronic displays comprising a plurality of pixels configured to produce a two-dimensional image;
   first and second imaging optics disposed respectively in first and second optical paths from said one or more electronic displays to form respective first and second collimated optical beams and images disposed at infinity; and
   an assistant display housing at least partially enclosing said displays and said imaging optics;
wherein said first and second imaging optics are configured to direct said first and second beams so that they are substantially parallel to each other and have cross-sections with centers separated from each other by between about 22 mm and 25 mm
(in some embodiments, the housing can include an opening, and the first and second imaging optics can be configured to direct the first and second beams through the opening, and the first and second beams can be substantially parallel to each other at the opening).

2. The medical apparatus of Embodiment 1, wherein said one or more electronic displays comprise first and second electronic displays.

3. The medical apparatus of Embodiment 1, wherein said one or more electronic displays comprise first and second portions of a single electronic display.

4. The medical apparatus of Embodiment 1, wherein said one or more electronic displays comprise at least one emissive display or at least one spatial light modulator.

5. The medical apparatus of Embodiment 1, wherein said one or more electronic displays comprise at least one liquid crystal display or at least one light emitting diode display.

6. The medical apparatus of Embodiment 1, further comprising a plurality of reflective surfaces in the optical paths of the optical beams to fold the optical beams.

7. The medical apparatus of Embodiment 6, wherein said plurality of reflective surfaces comprise mirrors.

8. The medical apparatus of Embodiment 6, wherein said plurality of reflective surfaces comprise between about 2 and 6 mirrors per side of said reflective surfaces in each of said first and second optical paths.

9. The medical apparatus of Embodiment 1, further comprising a plurality of reflective surfaces to fold the optical beams.

10. The medical apparatus of Embodiment 1, further comprising at least one mirror between said one or more electronic displays and said imaging optics.

11. The medical apparatus of Embodiment 1, further comprising between 0 and 2 mirrors between said one or more electronic displays and said imaging optics.

12. The medical apparatus of Embodiment 1, wherein said imaging optics comprise a plurality of lenses.

13. The medical apparatus of Embodiment 12, further comprising at least one mirror between said imaging optics and an exit pupil of said imaging optics.

14. The medical apparatus of Embodiment 13, wherein said at least one mirror comprises at least one mirror between said electronic display and said imaging optics, and at least one mirror disposed between lenses in said imaging optics, said former mirror being larger than said latter mirror.

15. The medical apparatus of Embodiment 1, wherein said imaging optics comprise between about 2 and 11 lenses.

16. The medical apparatus of Embodiment 1, wherein said imaging optics comprise positive lenses.

17. The medical apparatus of Embodiment 1, wherein said imaging optics have positive power.

18. The medical apparatus of Embodiment 1, wherein said imaging optics comprise a first lens configured to reduce a cross-section of the first beam.

19. The medical apparatus of Embodiment 18, wherein said first lens is configured to substantially collimate said first beam.

20. The medical apparatus of Embodiment 18, wherein said first lens has positive power.

21. The medical apparatus of Embodiment 18, wherein a lens in said imaging optics other than said first lens has an aperture size smaller than said first lens.

22. The medical apparatus of Embodiment 18, wherein lenses in said imaging optics other than said first lens have aperture sizes smaller than said first lens.

23. The medical apparatus of Embodiment 1, wherein said imaging optics is configured to magnify the display.

24. The medical apparatus of Embodiment 1, wherein said imaging optics has a field of view between about 3-10° 25. The medical apparatus of Embodiment 1, wherein said first and second imaging optics have exit pupils and said first and second electronic displays are not parallel to said exit pupils.

26. The medical apparatus of Embodiment 1, wherein said first and second imaging optics have exit pupils having centers and said first and second electronic displays have centers, said center of said exit pupils being displaced from said centers of said electronic displays.

27. The medical apparatus of Embodiment 1, wherein said first and second imaging optics have exit pupils and an optical path length from said one or more electronic displays to said exit pupils is between about 2 mm and 7 mm.

28. The medical apparatus of Embodiment 1, wherein an optical path length from said one or more electronic displays to said imaging optics is between about 100 mm and 400 mm.

29. The medical apparatus of Embodiment 1, wherein the imaging optics comprise a plurality of lenses including a first lens and a last lens in said optical paths and said imaging optics has an optical path length from said first lens to the last lens that is between about 50 mm and 250 mm.

30. The medical apparatus of Embodiment 1, wherein the imaging optics comprise a plurality of lenses including a first lens and an exit pupil in said optical paths and said imaging optics has an optical path length from said first lens to the exit pupil that is between about 10 mm and 50 mm.

31. The medical apparatus of Embodiment 1, wherein said one or more electronic displays comprise first and second electronic displays having centers spaced apart by a distance Wdisplay, wherein said first and second imaging optics have exit pupils having centers spaced apart by a distance Weyepaths, and wherein Wdisplay>Weyepaths.

32. The medical apparatus of Embodiment 1, wherein said one or more electronic displays comprise first and second electronic displays having centers spaced apart by a distance between about 100 mm and 200 mm.

33. The medical apparatus of Embodiment 1, wherein said first and second imaging optics have exit pupils having centers spaced apart by a distance of between about 22 mm and 25 mm.

34. The medical apparatus of Embodiment 1, wherein said first and second imaging optics have exit pupils having centers spaced apart by a distance of between about 50 mm and 200 mm over most of a distance through the imaging optics.

35. The medical apparatus of Embodiment 1, wherein said first and second imaging optics have optical axes spaced apart over most of a distance through the imaging optics by a distance of between about 50 mm and 200 mm.

36. The medical apparatus of Embodiment 1, wherein said first and second beams have cross-sections having centers spaced apart over most of a distance through the imaging optics by a distance of between about 15 mm and 35 mm.

37. The medical apparatus of Embodiment 1, wherein said first and second imaging optics have first and second exit pupils disposed a longitudinal distance along the length of the beam that is between about 0 mm and 45 mm from the opening.

38. The medical apparatus of Embodiment 1, wherein said housing has internal sidewalls darker than external sidewalls.

39. The medical apparatus of Embodiment 1, wherein said housing has dark internal sidewalls.

40. The medical apparatus of Embodiment 1, wherein said housing has black internal sidewalls.

41. The medical apparatus of Embodiment 1, further comprising baffles in said housing for reducing stray light.

42. The medical apparatus of Embodiment 1, wherein said opening comprises a mounting face configured to connect to a binocular assembly.

43. The medical apparatus of Embodiment 1, wherein said opening is between about 50 mm and 100 mm wide.

44. The medical apparatus of Embodiment 1, wherein said opening is circular.

45. The medical apparatus of Embodiment 1, wherein said opening is between about 66 mm and 70 mm in diameter.

46. The medical apparatus of Embodiment 1, wherein said opening comprises a mounting face having a size and shape configured to mate with a binocular assembly for a surgical microscope.

47. The medical apparatus of Embodiment 1, further comprising a binocular assembly comprising first and second objectives, first and second beam positioning optics, and first and second oculars.

48. The medical apparatus of Embodiment 47, wherein said binocular assembly has a magnification of between 8× and 13×.

49. The medical apparatus of Embodiment 47, wherein said binocular assembly has a magnification of between 10× and 12.5×.

50. The medical apparatus of Embodiment 47, wherein said imaging optics and said binocular assembly provide an apparent field of view of between 100-120°.

51. The medical apparatus of Embodiment 47, wherein said imaging optics and said binocular assembly provide an apparent field of view of about 110°.

52. The medical apparatus of Embodiment 47, wherein said imaging optics and said binocular assembly provide an apparent field of view of between 60-70º.

53. The medical apparatus of Embodiment 47, wherein said first and second beam positioning optics comprise prisms.

54. The medical apparatus of Embodiment 47, wherein said imaging optics have exit pupils having centers and said binocular assembly have entrance pupils having centers, and said centers of said entrance pupils are separated by a distance that is substantially the same as the separation between said centers of said exit pupils.

55. The medical apparatus of Embodiment 47, wherein said binocular assembly has entrance pupils having centers, and said centers of said entrance pupils are separated by a distance of between about 52 mm and 78 mm.

56. The medical apparatus of Embodiment 47, wherein said imaging optics have exit pupils and said binocular assembly have entrance pupils, and said entrance pupils are smaller than said exit pupils.

57. The medical apparatus of Embodiment 47, wherein said imaging optics have exit pupils and said binocular assembly have entrance pupils, and said entrance pupils are the same size as said exit pupils.

58. The medical apparatus of Embodiment 47, wherein said binocular assembly has entrance pupils, and said entrance pupils are 15 mm to 20 mm in diameter.

59. The medical apparatus of Embodiment 47, wherein said oculars on said binocular assembly have adjustable tilt to accommodate different heights of viewers.

60. The medical apparatus of Embodiment 47, wherein said binocular assembly has a housing with an opening and said opening is configured to interface with and connect to the opening of said an assistant display housing.

61. The medical apparatus of Embodiment 47, wherein said imaging optics have exit pupils disposed in an exit pupil plane and said binocular assembly have entrance pupils disposed in an entrance pupil plane, and said entrance pupil plane and said exit pupil plane are substantially coplanar.

62. The medical apparatus of Embodiment 47, wherein said entrance pupil plane and said exit pupil plane are separated by less than about 0 mm to 30 mm.

63. The medical apparatus of Embodiment 62, wherein said entrance pupil plane and said exit pupil plane are separated by less than 0 to 15 mm mm.

64. The medical apparatus of Embodiment 1, further comprising an articulated arm supporting said assistant display housing of said one or more electronic displays.

65. The medical apparatus of Embodiment 1, further comprising processing electronics configured to communicate with said one or more electronic displays to provide images for said one or more electronic displays.

66. The medical apparatus of Embodiment 65, wherein said electronics is configured to receive images from one or more cameras on a surgical device.

67. The medical apparatus of Embodiment 65, wherein said electronics is configured to receive images from one or more cameras on a surgical tool.

68. The medical apparatus of Embodiment 65, wherein said electronics is configured to receive images from one or more cameras on a surgical retractor.

69. The medical apparatus of Embodiment 65, wherein said electronics is configured to receive images from one or more cameras that provide a surgical microscope view.

70. The medical apparatus of Embodiment 69, wherein said camera is supported by an articulated arm that supports said assistant display housing for said electronic displays.

71. The medical apparatus of Embodiment 69, wherein said assistant display housing for said displays is supported by an articulated arm and said one or more cameras that provide a surgical view are supported by a separate platform that is configured to be able to be stationary with movement of said articulated arm.

72. The medical apparatus of Embodiment 65, wherein said first and second displays receive input images from said processing electronics corresponding respectively to left and right channels on a stereo camera and display said input images on said first and second electronic displays respectively.

73. The medical apparatus of Embodiment 72, further comprising a binocular assembly that receives said first and second beams from said first and second imaging optics and has oculars that output images from said first and second electronics display so as to render a three-dimensional image visible to a viewer peering through said oculars.

74. The medical apparatus of Embodiment 65, wherein said processing electronics are configured to receive images from memory that store previously recorded images.

75. The medical apparatus of Embodiment 65, wherein said processing electronics are configured to present images sources other than cameras.

76. The medical apparatus of Embodiment 65, wherein said processing electronics are configured to present images sources other than cameras in addition to images from cameras for simultaneous viewing by a viewer.

77. The medical apparatus of Embodiment 75 or 76, wherein said sources of images other than cameras comprises Computer Aided Tomography (CAT) scan, MRI, x-ray, and ultrasound imaging instruments.

78. The medical apparatus of Embodiment 75 or 76, wherein said source comprises a source of artificially generated image data.

79. The medical apparatus of Embodiment 1, further comprising at least one beam splitter disposed in one or both of said first and second optical paths configured to receiving images to be viewable by a binocular assembly connected to said assistant display housing in addition to images from said electronics displays.

80. The medical apparatus of Embodiment 79, further comprising at least one separate electronic display disposed with respect to said at least one beam splitter such that said one or both of said first and second optical paths receives images produced on said at least one electronic display through said at least one beam splitter for viewing through said binocular assembly connected to said housing in addition so images from said electronics displays.

81. The medical apparatus of Embodiment 79, wherein said at least one beam splitter comprises first and second beam splitters and said at least one separate electronic display comprises first and second displays configured to display a pair of two-dimensional images which together when viewed through said binocular assembly produces a three-dimensional image.

82. A surgical visualization system comprising:
a plurality of cameras configured to acquire video images of a surgical site, the plurality of cameras comprising at least two cameras configured to acquire video images within the surgical site;
an actuator configured to be actuated by a user of the surgical visualization device to deliver one or more user interface signals, wherein the actuator is not configured to be actuated by a hand of the user; and
an image processing system in communication with the plurality of cameras and the actuator, the image processing system comprising processing electronics,
wherein the image processing system is configured to:
   receive the video images acquired by the plurality of cameras;
   provide a plurality of output video images, each of the plurality of output video images based on video images acquired by a corresponding one of the plurality of cameras;
   present one of the plurality of output video images on a display; and
   present a different one of the plurality of output video images on the display in response to a user interface signal received from the actuator.

83. The surgical visualization system of Embodiment 82, wherein at least one of the plurality of output video images is represented by a reduced-size real-time video stream that is configured to be presented on a graphical user interface for selection by the user using the actuator.

84. The surgical visualization system of Embodiment 83, wherein the reduced-size real-time video stream comprises video from the respective camera.

85. The surgical visualization system of Embodiment 82, wherein output video images from at least one of the at least two cameras is provided in a thumbnail.

86. The surgical visualization system of Embodiment 86, wherein the actuator comprises a foot pedal.

87. The surgical visualization system of Embodiment 82, further comprising a second actuator.

88. The surgical visualization system of Embodiment 87, wherein the image processing system is further configured to resize the one of the plurality of output video images on the display in response to a user interface signal from the second actuator.

89. The surgical visualization system of Embodiment 82, wherein the image processing system is further configured to resize the one of the plurality of output video images on the display in response to a user interface signal from the actuator.

90. The surgical visualization system of Embodiment 82, wherein each of the plurality of output video images is represented by a reduced-size real-time video stream that is configured to be presented on a graphical user interface.

91. The surgical visualization system of Embodiment 90, wherein at least one of the plurality of output video images is displayed as a central video stream on the graphical user interface with the reduced-size real-time video streams arranged at different points on a periphery of the central video stream.

92. The surgical visualization system of Embodiment 91, wherein the image processing system is configured to switch which video stream is displayed as the central video stream on the graphical user interface in response to a user interface signal from the actuator.

93. The surgical visualization system of Embodiment 92, wherein the reduced-size real-time video streams are arranged on the periphery of the central video stream and correspond to a number such that a number of user interface signals received from the actuator indicates which of the reduced-size real-time video streams to display as the central video stream.

94. The surgical visualization system of Embodiment 90, wherein two or more of the plurality of output video images are displayed as central video streams on the graphical user interface.

95. The surgical visualization system of Embodiment 94, wherein a first one of the central video streams is presented overlaid on a second one of the central video streams, the second video stream having a larger size than the first video stream.

Various embodiments include cameras and display systems for displaying images (e.g., video) from video cameras on retractors, surgical tools, as well as video cameras (that are mounted on a binocular display nit or separate platform) that provide surgical microscope views or other patient views. In some embodiments the video cameras are located on surgical devices that are not retractors such as but not limited to endoscopes, laparoscopes, and arthroscopes. The surgical visualization system can switch among any and all of these sources as well as other sources of images and information so as to present any combination of said images and/or other information or said images and/or information alone. A switching module may be used to switch between the different cameras on different devices as well as obtain video or still images and/or information from elsewhere. Such images (e.g., video) can be displayed on the displays such as those described herein including stereo and mono. Oculars, as used herein, can be used for viewing left and right viewing portions and include eyepieces or other elements to accomplish such viewing. Accordingly, where the term ocular is used, unless explicitly stated otherwise, it is to be understood that a viewing assembly can be used, the viewing assembly configured to include left and right viewing portions.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate embodiments also can be implemented in combination in a single embodiment. Conversely, various features that are described in the context of a single embodiment also can be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

1.-182. (canceled)

183. A medical apparatus comprising:
a camera; and
a housing, the camera contained in the housing,
wherein the housing with the camera therein forms part of a unit that includes edges that fit into grooves such that the unit can be lowered or raised with respect to a retractor that is inserted within a surgical site formed by an incision in the body.

184. The medical apparatus of claim 183, wherein the camera is disposed at a distal end of the unit.

185. The medical apparatus of claim 183, wherein the camera is configured to be tilted.

186. The medical apparatus of claim 185, wherein the camera is configured to be tilted inward and downward into the surgical site.

187. The medical apparatus of claim 183, wherein the camera comprises a sensor and optics arranged with respect to the sensor.

188. The medical apparatus of claim 187, wherein the housing comprises a window through which an image can be taken via the optics and sensor.

189. The medical apparatus of claim 183, wherein the housing extends upward at a distal end of the housing.

190. The medical apparatus of claim 183, wherein the housing is thicker at a distal end.

191. The medical apparatus of claim 183, wherein the housing comprises smooth curves.

192. The medical apparatus of claim 183, further comprising a port configured to provide fluid to clean the camera.

193. The medical apparatus of claim 192, wherein the port is configured to provide liquid and air to clean the camera.

194. The medical apparatus of claim 183, wherein the edges are configured to fit into a groove of an insert that is inserted into a retractor.
